SEQUENCE LISTING (469 sequences)

SEQ ID NO: 1 - RNA, 3637 nt, Homo sapiens
  source 1..3637: mol_type = mRNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 2 - RNA, 21 nt, synthetic construct
  source 1..21: mol_type = other RNA
ggggatacctcaccaagatcc

SEQ ID NO: 3 - RNA, 21 nt, synthetic construct
  source 1..21: mol_type = other RNA
gggatacctcaccaagatcct

SEQ ID NO: 4 - RNA, 21 nt, synthetic construct
  source 1..21: mol_type = other RNA
ctcaccaagatcctgcatgtc

SEQ ID NO: 5 - RNA, 21 nt, synthetic construct
  source 1..21: mol_type = other RNA
caccaagatcctgcatgtctt

SEQ ID NO: 6 - RNA, 21 nt, synthetic construct
  source 1..21: mol_type = other RNA
ggtcatggtcaccgacttcga

SEQ ID NO: 7 - RNA, 21 nt, synthetic construct
  source 1..21: mol_type = other RNA
gcctggagtttattcggaaaa

SEQ ID NO: 8 - RNA, 21 nt, synthetic construct
  source 1..21: mol_type = other RNA
tcattggtgcctccagcgact

SEQ ID NO: 9 - RNA, 21 nt, synthetic construct
  source 1..21: mol_type = other RNA
tctctgccaaagatgtcatca

SEQ ID NO: 10 - RNA, 21 nt, synthetic construct
  source 1..21: mol_type = other RNA
tgccaggtgctgcctgctacc

SEQ ID NO: 11 - RNA, 21 nt, synthetic construct
  source 1..21: mol_type = other RNA
ggaacacagaccaggaagctc

SEQ ID NO: 12 - RNA, 21 nt, synthetic construct
  source 1..21: mol_type = other RNA
acccaagcaagcagacattta

SEQ ID NO: 13 - RNA, 21 nt, synthetic construct
  source 1..21: mol_type = other RNA
cccaagcaagcagacatttat

SEQ ID NO: 14 - RNA, 21 nt, synthetic construct
  source 1..21: mol_type = other RNA
caagcaagcagacatttatct

SEQ ID NO: 15 - RNA, 21 nt, synthetic construct
  source 1..21: mol_type = other RNA
agcagacatttatcttttggg

SEQ ID NO: 16 - RNA, 21 nt, synthetic construct
  source 1..21: mol_type = other RNA
tttatcttttgggtctgtcct

SEQ ID NO: 17 - RNA, 21 nt, synthetic construct
  source 1..21: mol_type = other RNA
tcttttgggtctgtcctctct

SEQ ID NO: 18 - RNA, 21 nt, synthetic construct
  source 1..21: mol_type = other RNA
cctctctgttgcctttttaca

SEQ ID NO: 19 - RNA, 21 nt, synthetic construct
  source 1..21: mol_type = other RNA
ctctctgttgcctttttacag

SEQ ID NO: 20 - RNA, 21 nt, synthetic construct
  source 1..21: mol_type = other RNA
tctctgttgcctttttacagc

SEQ ID NO: 21 - RNA, 21 nt, synthetic construct
  source 1..21: mol_type = other RNA
caacttttctagacctgtttt

SEQ ID NO: 22 - RNA, 21 nt, synthetic construct
  source 1..21: mol_type = other RNA
ctagacctgttttgcttttgt

SEQ ID NO: 23 - RNA, 21 nt, synthetic construct
  source 1..21: mol_type = other RNA
tagacctgttttgcttttgta

SEQ ID NO: 24 - RNA, 21 nt, synthetic construct
  source 1..21: mol_type = other RNA
gacctgttttgcttttgtaac

SEQ ID NO: 25 - RNA, 21 nt, synthetic construct
  source 1..21: mol_type = other RNA
cctgttttgcttttgtaactt

SEQ ID NO: 26 - RNA, 21 nt, synthetic construct
  source 1..21: mol_type = other RNA
ttttgcttttgtaacttgaag

SEQ ID NO: 27 - RNA, 21 nt, synthetic construct
  source 1..21: mol_type = other RNA
ttttgtaacttgaagatattt

SEQ ID NO: 28 - RNA, 21 nt, synthetic construct
  source 1..21: mol_type = other RNA
tttgtaacttgaagatattta

SEQ ID NO: 29 - RNA, 21 nt, synthetic construct
  source 1..21: mol_type = other RNA
ttgtaacttgaagatatttat

SEQ ID NO: 30 - RNA, 21 nt, synthetic construct
  source 1..21: mol_type = other RNA
ggatcttggtgaggtatcccc

SEQ ID NO: 31 - RNA, 21 nt, synthetic construct
  source 1..21: mol_type = other RNA
tgatcttggtgaggtatcccc

SEQ ID NO: 32 - RNA, 21 nt, synthetic construct
  source 1..21: mol_type = other RNA
agatcttggtgaggtatcccc

SEQ ID NO: 33 - RNA, 21 nt, synthetic construct
  source 1..21: mol_type = other RNA
ngatcttggtgaggtatcccc

SEQ ID NO: 34 - RNA, 21 nt, synthetic construct
  source 1..21: mol_type = other RNA
ngatcttggtgaggtatcccn

SEQ ID NO: 35 - RNA, 21 nt, synthetic construct
  source 1..21: mol_type = other RNA
aggatcttggtgaggtatccc

SEQ ID NO: 36 - RNA, 21 nt, synthetic construct
  source 1..21: mol_type = other RNA
tggatcttggtgaggtatccc

SEQ ID NO: 37 - RNA, 21 nt, synthetic construct
  source 1..21: mol_type = other RNA
nggatcttggtgaggtatccc

SEQ ID NO: 38 - RNA, 21 nt, synthetic construct
  source 1..21: mol_type = other RNA
nggatcttggtgaggtatccn

SEQ ID NO: 39 - RNA, 21 nt, synthetic construct
  source 1..21: mol_type = other RNA
gacatgcaggatcttggtgag

SEQ ID NO: 40 - RNA, 21 nt, synthetic construct
  source 1..21: mol_type = other RNA
tacatgcaggatcttggtgag

SEQ ID NO: 41 - RNA, 21 nt, synthetic construct
  source 1..21: mol_type = other RNA
aacatgcaggatcttggtgag

SEQ ID NO: 42 - RNA, 21 nt, synthetic construct
  source 1..21: mol_type = other RNA
nacatgcaggatcttggtgag

SEQ ID NO: 43 - RNA, 21 nt, synthetic construct
  source 1..21: mol_type = other RNA
nacatgcaggatcttggtgan

SEQ ID NO: 44 - RNA, 21 nt, synthetic construct
  source 1..21: mol_type = other RNA
aagacatgcaggatcttggtg

SEQ ID NO: 45 - RNA, 21 nt, synthetic construct
  source 1..21: mol_type = other RNA
tagacatgcaggatcttggtg

SEQ ID NO: 46 - RNA, 21 nt, synthetic construct
  source 1..21: mol_type = other RNA
nagacatgcaggatcttggtg

SEQ ID NO: 47 - RNA, 21 nt, synthetic construct
  source 1..21: mol_type = other RNA
nagacatgcaggatcttggtn

SEQ ID NO: 48 - RNA, 21 nt, synthetic construct
  source 1..21: mol_type = other RNA
tcgaagtcggtgaccatgacc

SEQ ID NO: 49 - RNA, 21 nt, synthetic construct
  source 1..21: mol_type = other RNA
acgaagtcggtgaccatgacc

SEQ ID NO: 50 - RNA, 21 nt, synthetic construct
  source 1..21: mol_type = other RNA
ncgaagtcggtgaccatgacc

SEQ ID NO: 51 - RNA, 21 nt, synthetic construct
  source 1..21: mol_type = other RNA
ncgaagtcggtgaccatgacn

SEQ ID NO: 52 - RNA, 21 nt, synthetic construct
  source 1..21: mol_type = other RNA
ttttccgaataaactccaggc

SEQ ID NO: 53 - RNA, 21 nt, synthetic construct
  source 1..21: mol_type = other RNA
atttccgaataaactccaggc

SEQ ID NO: 54 - RNA, 21 nt, synthetic construct
  source 1..21: mol_type = other RNA
ntttccgaataaactccaggc

SEQ ID NO: 55 - RNA, 21 nt, synthetic construct
  source 1..21: mol_type = other RNA
ntttccgaataaactccaggn

SEQ ID NO: 56 - RNA, 21 nt, synthetic construct
  source 1..21: mol_type = other RNA
agtcgctggaggcaccaatga

SEQ ID NO: 57 - RNA, 21 nt, synthetic construct
  source 1..21: mol_type = other RNA
tgtcgctggaggcaccaatga

SEQ ID NO: 58 - RNA, 21 nt, synthetic construct
  source 1..21: mol_type = other RNA
ngtcgctggaggcaccaatga

SEQ ID NO: 59 - RNA, 21 nt, synthetic construct
  source 1..21: mol_type = other RNA
ngtcgctggaggcaccaatgn

SEQ ID NO: 60 - RNA, 21 nt, synthetic construct
  source 1..21: mol_type = other RNA
tgatgacatctttggcagaga

SEQ ID NO: 61 - RNA, 21 nt, synthetic construct
  source 1..21: mol_type = other RNA
agatgacatctttggcagaga

SEQ ID NO: 62 - RNA, 21 nt, synthetic construct
  source 1..21: mol_type = other RNA
ngatgacatctttggcagaga

SEQ ID NO: 63 - RNA, 21 nt, synthetic construct
  source 1..21: mol_type = other RNA
ngatgacatctttggcagagn

SEQ ID NO: 64 - RNA, 21 nt, synthetic construct
  source 1..21: mol_type = other RNA
ggtagcaggcagcacctggca

SEQ ID NO: 65 - RNA, 21 nt, synthetic construct
  source 1..21: mol_type = other RNA
tgtagcaggcagcacctggca

SEQ ID NO: 66 - RNA, 21 nt, synthetic construct
  source 1..21: mol_type = other RNA
agtagcaggcagcacctggca

SEQ ID NO: 67 - RNA, 21 nt, synthetic construct
  source 1..21: mol_type = other RNA
ngtagcaggcagcacctggca

SEQ ID NO: 68 - RNA, 21 nt, synthetic construct
  source 1..21: mol_type = other RNA
ngtagcaggcagcacctggcn

SEQ ID NO: 69 - RNA, 21 nt, synthetic construct
  source 1..21: mol_type = other RNA
gagcttcctggtctgtgttcc

SEQ ID NO: 70 - RNA, 21 nt, synthetic construct
  source 1..21: mol_type = other RNA
tagcttcctggtctgtgttcc

SEQ ID NO: 71 - RNA, 21 nt, synthetic construct
  source 1..21: mol_type = other RNA
aagcttcctggtctgtgttcc

SEQ ID NO: 72 - RNA, 21 nt, synthetic construct
  source 1..21: mol_type = other RNA
nagcttcctggtctgtgttcc

SEQ ID NO: 73 - RNA, 21 nt, synthetic construct
  source 1..21: mol_type = other RNA
nagcttcctggtctgtgttcn

SEQ ID NO: 74 - RNA, 21 nt, synthetic construct
  source 1..21: mol_type = other RNA
taaatgtctgcttgcttgggt

SEQ ID NO: 75 - RNA, 21 nt, synthetic construct
  source 1..21: mol_type = other RNA
aaaatgtctgcttgcttgggt

SEQ ID NO: 76 - RNA, 21 nt, synthetic construct
  source 1..21: mol_type = other RNA
naaatgtctgcttgcttgggt

SEQ ID NO: 77 - RNA, 21 nt, synthetic construct
  source 1..21: mol_type = other RNA
naaatgtctgcttgcttgggn

SEQ ID NO: 78 - RNA, 21 nt, synthetic construct
  source 1..21: mol_type = other RNA
ataaatgtctgcttgcttggg

SEQ ID NO: 79 - RNA, 21 nt, synthetic construct
  source 1..21: mol_type = other RNA
ttaaatgtctgcttgcttggg

SEQ ID NO: 80 - RNA, 21 nt, synthetic construct
  source 1..21: mol_type = other RNA
ntaaatgtctgcttgcttggg

SEQ ID NO: 81 - RNA, 21 nt, synthetic construct
  source 1..21: mol_type = other RNA
ntaaatgtctgcttgcttggn

SEQ ID NO: 82 - RNA, 21 nt, synthetic construct
  source 1..21: mol_type = other RNA
agataaatgtctgcttgcttg

SEQ ID NO: 83 - RNA, 21 nt, synthetic construct
  source 1..21: mol_type = other RNA
tgataaatgtctgcttgcttg

SEQ ID NO: 84 - RNA, 21 nt, synthetic construct
  source 1..21: mol_type = other RNA
ngataaatgtctgcttgcttg

SEQ ID NO: 85 - RNA, 21 nt, synthetic construct
  source 1..21: mol_type = other RNA
ngataaatgtctgcttgcttn

SEQ ID NO: 86 - RNA, 21 nt, synthetic construct
  source 1..21: mol_type = other RNA
cccaaaagataaatgtctgct

SEQ ID NO: 87 - RNA, 21 nt, synthetic construct
  source 1..21: mol_type = other RNA
tccaaaagataaatgtctgct

SEQ ID NO: 88 - RNA, 21 nt, synthetic construct
  source 1..21: mol_type = other RNA
accaaaagataaatgtctgct

SEQ ID NO: 89 - RNA, 21 nt, synthetic construct
  source 1..21: mol_type = other RNA
nccaaaagataaatgtctgct

SEQ ID NO: 90 - RNA, 21 nt, synthetic construct
  source 1..21: mol_type = other RNA
nccaaaagataaatgtctgcn

SEQ ID NO: 91 - RNA, 21 nt, synthetic construct
  source 1..21: mol_type = other RNA
aggacagacccaaaagataaa

SEQ ID NO: 92 - RNA, 21 nt, synthetic construct
  source 1..21: mol_type = other RNA
tggacagacccaaaagataaa

SEQ ID NO: 93 - RNA, 21 nt, synthetic construct
  source 1..21: mol_type = other RNA
nggacagacccaaaagataaa

SEQ ID NO: 94 - RNA, 21 nt, synthetic construct
  source 1..21: mol_type = other RNA
nggacagacccaaaagataan

SEQ ID NO: 95 - DNA, 21 bp, synthetic construct
  source 1..21: mol_type = other DNA
agagaggacagacccaaaaga

SEQ ID NO: 96 - RNA, 21 nt, synthetic construct
  source 1..21: mol_type = other RNA
tgagaggacagacccaaaaga

SEQ ID NO: 97 - DNA, 21 bp, synthetic construct
  source 1..21: mol_type = other DNA
ngagaggacagacccaaaaga

SEQ ID NO: 98 - DNA, 21 bp, synthetic construct
  source 1..21: mol_type = other DNA
ngagaggacagacccaaaagn

SEQ ID NO: 99 - RNA, 21 nt, synthetic construct
  source 1..21: mol_type = other RNA
tgtaaaaaggcaacagagagg

SEQ ID NO: 100 - RNA, 21 nt, synthetic construct
  source 1..21: mol_type = other RNA
agtaaaaaggcaacagagagg

SEQ ID NO: 101 - RNA, 21 nt, synthetic construct
  source 1..21: mol_type = other RNA
ngtaaaaaggcaacagagagg

SEQ ID NO: 102 - RNA, 21 nt, synthetic construct
  source 1..21: mol_type = other RNA
ngtaaaaaggcaacagagagn

SEQ ID NO: 103 - RNA, 21 nt, synthetic construct
  source 1..21: mol_type = other RNA
ctgtaaaaaggcaacagagag

SEQ ID NO: 104 - RNA, 21 nt, synthetic construct
  source 1..21: mol_type = other RNA
ttgtaaaaaggcaacagagag

SEQ ID NO: 105 - RNA, 21 nt, synthetic construct
  source 1..21: mol_type = other RNA
atgtaaaaaggcaacagagag

SEQ ID NO: 106 - RNA, 21 nt, synthetic construct
  source 1..21: mol_type = other RNA
ntgtaaaaaggcaacagagag

SEQ ID NO: 107 - RNA, 21 nt, synthetic construct
  source 1..21: mol_type = other RNA
ntgtaaaaaggcaacagagan

SEQ ID NO: 108 - RNA, 21 nt, synthetic construct
  source 1..21: mol_type = other RNA
gctgtaaaaaggcaacagaga

SEQ ID NO: 109 - RNA, 21 nt, synthetic construct
  source 1..21: mol_type = other RNA
tctgtaaaaaggcaacagaga

SEQ ID NO: 110 - RNA, 21 nt, synthetic construct
  source 1..21: mol_type = other RNA
actgtaaaaaggcaacagaga

SEQ ID NO: 111 - RNA, 21 nt, synthetic construct
  source 1..21: mol_type = other RNA
nctgtaaaaaggcaacagaga

SEQ ID NO: 112 - RNA, 21 nt, synthetic construct
  source 1..21: mol_type = other RNA
nctgtaaaaaggcaacagagn

SEQ ID NO: 113 - RNA, 21 nt, synthetic construct
  source 1..21: mol_type = other RNA
aaaacaggtctagaaaagttg

SEQ ID NO: 114 - RNA, 21 nt, synthetic construct
  source 1..21: mol_type = other RNA
taaacaggtctagaaaagttg

SEQ ID NO: 115 - RNA, 21 nt, synthetic construct
  source 1..21: mol_type = other RNA
naaacaggtctagaaaagttg

SEQ ID NO: 116 - RNA, 21 nt, synthetic construct
  source 1..21: mol_type = other RNA
naaacaggtctagaaaagttn

SEQ ID NO: 117 - RNA, 21 nt, synthetic construct
  source 1..21: mol_type = other RNA
acaaaagcaaaacaggtctag

SEQ ID NO: 118 - RNA, 21 nt, synthetic construct
  source 1..21: mol_type = other RNA
tcaaaagcaaaacaggtctag

SEQ ID NO: 119 - RNA, 21 nt, synthetic construct
  source 1..21: mol_type = other RNA
ncaaaagcaaaacaggtctag

SEQ ID NO: 120 - RNA, 21 nt, synthetic construct
  source 1..21: mol_type = other RNA
ncaaaagcaaaacaggtctan

SEQ ID NO: 121 - RNA, 21 nt, synthetic construct
  source 1..21: mol_type = other RNA
tacaaaagcaaaacaggtcta

SEQ ID NO: 122 - RNA, 21 nt, synthetic construct
  source 1..21: mol_type = other RNA
aacaaaagcaaaacaggtcta

SEQ ID NO: 123 - RNA, 21 nt, synthetic construct
  source 1..21: mol_type = other RNA
nacaaaagcaaaacaggtcta

SEQ ID NO: 124 - RNA, 21 nt, synthetic construct
  source 1..21: mol_type = other RNA
nacaaaagcaaaacaggtctn

SEQ ID NO: 125 - RNA, 21 nt, synthetic construct
  source 1..21: mol_type = other RNA
gttacaaaagcaaaacaggtc

SEQ ID NO: 126 - RNA, 21 nt, synthetic construct
  source 1..21: mol_type = other RNA
tttacaaaagcaaaacaggtc

SEQ ID NO: 127 - RNA, 21 nt, synthetic construct
  source 1..21: mol_type = other RNA
attacaaaagcaaaacaggtc

SEQ ID NO: 128 - RNA, 21 nt, synthetic construct
  source 1..21: mol_type = other RNA
nttacaaaagcaaaacaggtc

SEQ ID NO: 129 - RNA, 21 nt, synthetic construct
  source 1..21: mol_type = other RNA
nttacaaaagcaaaacaggtn

SEQ ID NO: 130 - RNA, 21 nt, synthetic construct
  source 1..21: mol_type = other RNA
aagttacaaaagcaaaacagg

SEQ ID NO: 131 - RNA, 21 nt, synthetic construct
  source 1..21: mol_type = other RNA
tagttacaaaagcaaaacagg

SEQ ID NO: 132 - RNA, 21 nt, synthetic construct
  source 1..21: mol_type = other RNA
nagttacaaaagcaaaacagg

SEQ ID NO: 133 - RNA, 21 nt, synthetic construct
  source 1..21: mol_type = other RNA
aagttacaaaagcaaaacagn

SEQ ID NO: 134 - RNA, 21 nt, synthetic construct
  source 1..21: mol_type = other RNA
nagttacaaaagcaaaacagn

SEQ ID NO: 135 - RNA, 21 nt, synthetic construct
  source 1..21: mol_type = other RNA
aagttacaaaagcaaaacann

SEQ ID NO: 136 - RNA, 21 nt, synthetic construct
  source 1..21: mol_type = other RNA
tagttacaaaagcaaaacann

SEQ ID NO: 137 - RNA, 21 nt, synthetic construct
  source 1..21: mol_type = other RNA
nagttacaaaagcaaaacann

SEQ ID NO: 138 - RNA, 21 nt, synthetic construct
  source 1..21: mol_type = other RNA
aagttacaaaagcaaaacnnn

SEQ ID NO: 139 - RNA, 21 nt, synthetic construct
  source 1..21: mol_type = other RNA
tagttacaaaagcaaaacnnn

SEQ ID NO: 140 - RNA, 21 nt, synthetic construct
  source 1..21: mol_type = other RNA
nagttacaaaagcaaaacnnn

SEQ ID NO: 141 - RNA, 21 nt, synthetic construct
  source 1..21: mol_type = other RNA
cttcaagttacaaaagcaaaa

SEQ ID NO: 142 - RNA, 21 nt, synthetic construct
  source 1..21: mol_type = other RNA
tttcaagttacaaaagcaaaa

SEQ ID NO: 143 - RNA, 21 nt, synthetic construct
  source 1..21: mol_type = other RNA
attcaagttacaaaagcaaaa

SEQ ID NO: 144 - RNA, 21 nt, synthetic construct
  source 1..21: mol_type = other RNA
nttcaagttacaaaagcaaaa

SEQ ID NO: 145 - RNA, 21 nt, synthetic construct
  source 1..21: mol_type = other RNA
nttcaagttacaaaagcaaan

SEQ ID NO: 146 - RNA, 21 nt, synthetic construct
  source 1..21: mol_type = other RNA
aaatatcttcaagttacaaaa

SEQ ID NO: 147 - RNA, 21 nt, synthetic construct
  source 1..21: mol_type = other RNA
taatatcttcaagttacaaaa

SEQ ID NO: 148 - RNA, 21 nt, synthetic construct
  source 1..21: mol_type = other RNA
naatatcttcaagttacaaaa

SEQ ID NO: 149 - RNA, 21 nt, synthetic construct
  source 1..21: mol_type = other RNA
naatatcttcaagttacaaan

SEQ ID NO: 150 - RNA, 21 nt, synthetic construct
  source 1..21: mol_type = other RNA
taaatatcttcaagttacaaa

SEQ ID NO: 151 - RNA, 21 nt, synthetic construct
  source 1..21: mol_type = other RNA
aaaatatcttcaagttacaaa

SEQ ID NO: 152 - RNA, 21 nt, synthetic construct
  source 1..21: mol_type = other RNA
naaatatcttcaagttacaaa

SEQ ID NO: 153 - RNA, 21 nt, synthetic construct
  source 1..21: mol_type = other RNA
naaatatcttcaagttacaan

SEQ ID NO: 154 - RNA, 21 nt, synthetic construct
  source 1..21: mol_type = other RNA
ataaatatcttcaagttacaa

SEQ ID NO: 155 - RNA, 21 nt, synthetic construct
  source 1..21: mol_type = other RNA
ttaaatatcttcaagttacaa

SEQ ID NO: 156 - RNA, 21 nt, synthetic construct
  source 1..21: mol_type = other RNA
ntaaatatcttcaagttacaa

SEQ ID NO: 157 - RNA, 21 nt, synthetic construct
  source 1..21: mol_type = other RNA
ntaaatatcttcaagttacan

SEQ ID NO: 158 - RNA, 21 nt, synthetic construct
  source 1..21: mol_type = other RNA
ggggatacctcaccaagatcc

SEQ ID NO: 159 - RNA, 21 nt, synthetic construct
  source 1..21: mol_type = other RNA
ggggatacctcaccaagatca

SEQ ID NO: 160 - RNA, 21 nt, synthetic construct
  source 1..21: mol_type = other RNA
ggggatacctcaccaagatct

SEQ ID NO: 161 - RNA, 21 nt, synthetic construct
  source 1..21: mol_type = other RNA
ggggatacctcaccaagatcn

SEQ ID NO: 162 - RNA, 21 nt, synthetic construct
  source 1..21: mol_type = other RNA
ngggatacctcaccaagatcn

SEQ ID NO: 163 - RNA, 21 nt, synthetic construct
  source 1..21: mol_type = other RNA
gggatacctcaccaagatcct

SEQ ID NO: 164 - RNA, 21 nt, synthetic construct
  source 1..21: mol_type = other RNA
gggatacctcaccaagatcca

SEQ ID NO: 165 - RNA, 21 nt, synthetic construct
  source 1..21: mol_type = other RNA
gggatacctcaccaagatccn

SEQ ID NO: 166 - RNA, 21 nt, synthetic construct
  source 1..21: mol_type = other RNA
nggatacctcaccaagatccn

SEQ ID NO: 167 - RNA, 21 nt, synthetic construct
  source 1..21: mol_type = other RNA
ctcaccaagatcctgcatgtc

SEQ ID NO: 168 - RNA, 21 nt, synthetic construct
  source 1..21: mol_type = other RNA
ctcaccaagatcctgcatgta

SEQ ID NO: 169 - RNA, 21 nt, synthetic construct
  source 1..21: mol_type = other RNA
ctcaccaagatcctgcatgtt

SEQ ID NO: 170 - RNA, 21 nt, synthetic construct
  source 1..21: mol_type = other RNA
ctcaccaagatcctgcatgtn

SEQ ID NO: 171 - RNA, 21 nt, synthetic construct
  source 1..21: mol_type = other RNA
ntcaccaagatcctgcatgtn

SEQ ID NO: 172 - RNA, 21 nt, synthetic construct
  source 1..21: mol_type = other RNA
caccaagatcctgcatgtctt

SEQ ID NO: 173 - RNA, 21 nt, synthetic construct
  source 1..21: mol_type = other RNA
caccaagatcctgcatgtcta

SEQ ID NO: 174 - RNA, 21 nt, synthetic construct
  source 1..21: mol_type = other RNA
caccaagatcctgcatgtctn

SEQ ID NO: 175 - RNA, 21 nt, synthetic construct
  source 1..21: mol_type = other RNA
naccaagatcctgcatgtctn

SEQ ID NO: 176 - RNA, 21 nt, synthetic construct
  source 1..21: mol_type = other RNA
ggtcatggtcaccgacttcga

SEQ ID NO: 177 - RNA, 21 nt, synthetic construct
  source 1..21: mol_type = other RNA
ggtcatggtcaccgacttcgt

SEQ ID NO: 178 - RNA, 21 nt, synthetic construct
  source 1..21: mol_type = other RNA
ggtcatggtcaccgacttcgn

SEQ ID NO: 179 - RNA, 21 nt, synthetic construct
  source 1..21: mol_type = other RNA
ngtcatggtcaccgacttcgn

SEQ ID NO: 180 - RNA, 21 nt, synthetic construct
  source 1..21: mol_type = other RNA
gcctggagtttattcggaaaa

SEQ ID NO: 181 - RNA, 21 nt, synthetic construct
  source 1..21: mol_type = other RNA
gcctggagtttattcggaaat

SEQ ID NO: 182 - RNA, 21 nt, synthetic construct
  source 1..21: mol_type = other RNA
gcctggagtttattcggaaan

SEQ ID NO: 183 - RNA, 21 nt, synthetic construct
  source 1..21: mol_type = other RNA
ncctggagtttattcggaaan

SEQ ID NO: 184 - RNA, 21 nt, synthetic construct
  source 1..21: mol_type = other RNA
tcattggtgcctccagcgact

SEQ ID NO: 185 - RNA, 21 nt, synthetic construct
  source 1..21: mol_type = other RNA
tcattggtgcctccagcgaca

SEQ ID NO: 186 - RNA, 21 nt, synthetic construct
  source 1..21: mol_type = other RNA
tcattggtgcctccagcgacn

SEQ ID NO: 187 - RNA, 21 nt, synthetic construct
  source 1..21: mol_type = other RNA
ncattggtgcctccagcgacn

SEQ ID NO: 188 - RNA, 21 nt, synthetic construct
  source 1..21: mol_type = other RNA
tctctgccaaagatgtcatca

SEQ ID NO: 189 - RNA, 21 nt, synthetic construct
  source 1..21: mol_type = other RNA
tctctgccaaagatgtcatct

SEQ ID NO: 190 - RNA, 21 nt, synthetic construct
  source 1..21: mol_type = other RNA
tctctgccaaagatgtcatcn

SEQ ID NO: 191 - RNA, 21 nt, synthetic construct
  source 1..21: mol_type = other RNA
nctctgccaaagatgtcatcn

SEQ ID NO: 192 - RNA, 21 nt, synthetic construct
  source 1..21: mol_type = other RNA
tgccaggtgctgcctgctacc

SEQ ID NO: 193 - RNA, 21 nt, synthetic construct
  source 1..21: mol_type = other RNA
tgccaggtgctgcctgctaca

SEQ ID NO: 194 - RNA, 21 nt, synthetic construct
  source 1..21: mol_type = other RNA
tgccaggtgctgcctgctact

SEQ ID NO: 195 - RNA, 21 nt, synthetic construct
  source 1..21: mol_type = other RNA
tgccaggtgctgcctgctacn

SEQ ID NO: 196 - RNA, 21 nt, synthetic construct
  source 1..21: mol_type = other RNA
ngccaggtgctgcctgctacn

SEQ ID NO: 197 - RNA, 21 nt, synthetic construct
  source 1..21: mol_type = other RNA
ggaacacagaccaggaagctc

SEQ ID NO: 198 - RNA, 21 nt, synthetic construct
  source 1..21: mol_type = other RNA
ggaacacagaccaggaagcta

SEQ ID NO: 199 - RNA, 21 nt, synthetic construct
  source 1..21: mol_type = other RNA
ggaacacagaccaggaagctt

SEQ ID NO: 200 - RNA, 21 nt, synthetic construct
  source 1..21: mol_type = other RNA
ggaacacagaccaggaagctn

SEQ ID NO: 201 - RNA, 21 nt, synthetic construct
  source 1..21: mol_type = other RNA
ngaacacagaccaggaagctn

SEQ ID NO: 202 - RNA, 21 nt, synthetic construct
  source 1..21: mol_type = other RNA
acccaagcaagcagacattta

SEQ ID NO: 203 - RNA, 21 nt, synthetic construct
  source 1..21: mol_type = other RNA
acccaagcaagcagacatttt

SEQ ID NO: 204 - RNA, 21 nt, synthetic construct
  source 1..21: mol_type = other RNA
acccaagcaagcagacatttn

SEQ ID NO: 205 - RNA, 21 nt, synthetic construct
  source 1..21: mol_type = other RNA
ncccaagcaagcagacatttn

SEQ ID NO: 206 - RNA, 21 nt, synthetic construct
  source 1..21: mol_type = other RNA
cccaagcaagcagacatttat

SEQ ID NO: 207 - RNA, 21 nt, synthetic construct
  source 1..21: mol_type = other RNA
cccaagcaagcagacatttaa

SEQ ID NO: 208 - RNA, 21 nt, synthetic construct
  source 1..21: mol_type = other RNA
cccaagcaagcagacatttan

SEQ ID NO: 209 - RNA, 21 nt, synthetic construct
  source 1..21: mol_type = other RNA
nccaagcaagcagacatttan

SEQ ID NO: 210 - RNA, 21 nt, synthetic construct
  source 1..21: mol_type = other RNA
caagcaagcagacatttatct

SEQ ID NO: 211 - RNA, 21 nt, synthetic construct
  source 1..21: mol_type = other RNA
caagcaagcagacatttatca

SEQ ID NO: 212 - RNA, 21 nt, synthetic construct
  source 1..21: mol_type = other RNA
caagcaagcagacatttatcn

SEQ ID NO: 213 - RNA, 21 nt, synthetic construct
  source 1..21: mol_type = other RNA
naagcaagcagacatttatcn

SEQ ID NO: 214 - RNA, 21 nt, synthetic construct
  source 1..21: mol_type = other RNA
agcagacatttatcttttggg

SEQ ID NO: 215 - RNA, 21 nt, synthetic construct
  source 1..21: mol_type = other RNA
agcagacatttatcttttgga

SEQ ID NO: 216 - RNA, 21 nt, synthetic construct
  source 1..21: mol_type = other RNA
agcagacatttatcttttggt

SEQ ID NO: 217 - RNA, 21 nt, synthetic construct
  source 1..21: mol_type = other RNA
agcagacatttatcttttggn

SEQ ID NO: 218 - RNA, 21 nt, synthetic construct
  source 1..21: mol_type = other RNA
ngcagacatttatcttttggn

SEQ ID NO: 219 - RNA, 21 nt, synthetic construct
  source 1..21: mol_type = other RNA
tttatcttttgggtctgtcct

SEQ ID NO: 220 - RNA, 21 nt, synthetic construct
  source 1..21: mol_type = other RNA
tttatcttttgggtctgtcca

SEQ ID NO: 221 - RNA, 21 nt, synthetic construct
  source 1..21: mol_type = other RNA
tttatcttttgggtctgtccn

SEQ ID NO: 222 - RNA, 21 nt, synthetic construct
  source 1..21: mol_type = other RNA
nttatcttttgggtctgtccn

SEQ ID NO: 223 - RNA, 21 nt, synthetic construct
  source 1..21: mol_type = other RNA
tcttttgggtctgtcctctct

SEQ ID NO: 224 - RNA, 21 nt, synthetic construct
  source 1..21: mol_type = other RNA
tcttttgggtctgtcctctca

SEQ ID NO: 225 - RNA, 21 nt, synthetic construct
  source 1..21: mol_type = other RNA
tcttttgggtctgtcctctcn

SEQ ID NO: 226 - RNA, 21 nt, synthetic construct
  source 1..21: mol_type = other RNA
ncttttgggtctgtcctctcn

SEQ ID NO: 227 - RNA, 21 nt, synthetic construct
  source 1..21: mol_type = other RNA
cctctctgttgcctttttaca

SEQ ID NO: 228 - RNA, 21 nt, synthetic construct
  source 1..21: mol_type = other RNA
cctctctgttgcctttttact

SEQ ID NO: 229 - RNA, 21 nt, synthetic construct
  source 1..21: mol_type = other RNA
cctctctgttgcctttttacn

SEQ ID NO: 230 - RNA, 21 nt, synthetic construct
  source 1..21: mol_type = other RNA
nctctctgttgcctttttacn

SEQ ID NO: 231 - RNA, 21 nt, synthetic construct
  source 1..21: mol_type = other RNA
ctctctgttgcctttttacag

SEQ ID NO: 232 - RNA, 21 nt, synthetic construct
  source 1..21: mol_type = other RNA
ctctctgttgcctttttacaa

SEQ ID NO: 233 - RNA, 21 nt, synthetic construct
  source 1..21: mol_type = other RNA
ctctctgttgcctttttacat

SEQ ID NO: 234 - RNA, 21 nt, synthetic construct
  source 1..21: mol_type = other RNA
ctctctgttgcctttttacan

SEQ ID NO: 235 - RNA, 21 nt, synthetic construct
  source 1..21: mol_type = other RNA
ntctctgttgcctttttacan

SEQ ID NO: 236 - RNA, 21 nt, synthetic construct
  source 1..21: mol_type = other RNA
tctctgttgcctttttacagc

SEQ ID NO: 237 - RNA, 21 nt, synthetic construct
  source 1..21: mol_type = other RNA
tctctgttgcctttttacaga

SEQ ID NO: 238 - RNA, 21 nt, synthetic construct
  source 1..21: mol_type = other RNA
tctctgttgcctttttacagt

SEQ ID NO: 239 - RNA, 21 nt, synthetic construct
  source 1..21: mol_type = other RNA
tctctgttgcctttttacagn

SEQ ID NO: 240 - RNA, 21 nt, synthetic construct
  source 1..21: mol_type = other RNA
nctctgttgcctttttacagn

SEQ ID NO: 241 - RNA, 21 nt, synthetic construct
  source 1..21: mol_type = other RNA
caacttttctagacctgtttt

SEQ ID NO: 242 - RNA, 21 nt, synthetic construct
  source 1..21: mol_type = other RNA
caacttttctagacctgttta

SEQ ID NO: 243 - RNA, 21 nt, synthetic construct
  source 1..21: mol_type = other RNA
caacttttctagacctgtttn

SEQ ID NO: 244 - RNA, 21 nt, synthetic construct
  source 1..21: mol_type = other RNA
naacttttctagacctgtttn

SEQ ID NO: 245 - RNA, 21 nt, synthetic construct
  source 1..21: mol_type = other RNA
ctagacctgttttgcttttgt

SEQ ID NO: 246 - RNA, 21 nt, synthetic construct
  source 1..21: mol_type = other RNA
ctagacctgttttgcttttga

SEQ ID NO: 247 - RNA, 21 nt, synthetic construct
  source 1..21: mol_type = other RNA
ctagacctgttttgcttttgn

SEQ ID NO: 248 - RNA, 21 nt, synthetic construct
  source 1..21: mol_type = other RNA
ntagacctgttttgcttttgn

SEQ ID NO: 249 - RNA, 21 nt, synthetic construct
  source 1..21: mol_type = other RNA
tagacctgttttgcttttgta

SEQ ID NO: 250 - RNA, 21 nt, synthetic construct
  source 1..21: mol_type = other RNA
tagacctgttttgcttttgtt

SEQ ID NO: 251 - RNA, 21 nt, synthetic construct
  source 1..21: mol_type = other RNA
tagacctgttttgcttttgtn

SEQ ID NO: 252 - RNA, 21 nt, synthetic construct
  source 1..21: mol_type = other RNA
nagacctgttttgcttttgtn

SEQ ID NO: 253 - RNA, 21 nt, synthetic construct
  source 1..21: mol_type = other RNA
gacctgttttgcttttgtaac

SEQ ID NO: 254 - RNA, 21 nt, synthetic construct
  source 1..21: mol_type = other RNA
gacctgttttgcttttgtaaa

SEQ ID NO: 255 - RNA, 21 nt, synthetic construct
  source 1..21: mol_type = other RNA
gacctgttttgcttttgtaat

SEQ ID NO: 256 - RNA, 21 nt, synthetic construct
  source 1..21: mol_type = other RNA
gacctgttttgcttttgtaan

SEQ ID NO: 257 - RNA, 21 nt, synthetic construct
  source 1..21: mol_type = other RNA
nacctgttttgcttttgtaan

SEQ ID NO: 258 - RNA, 21 nt, synthetic construct
  source 1..21: mol_type = other RNA
cctgttttgcttttgtaactt

SEQ ID NO: 259 - RNA, 21 nt, synthetic construct
  source 1..21: mol_type = other RNA
cctgttttgcttttgtaacta

SEQ ID NO: 260 - RNA, 21 nt, synthetic construct
  source 1..21: mol_type = other RNA
cctgttttgcttttgtaactn

SEQ ID NO: 261 - RNA, 21 nt, synthetic construct
  source 1..21: mol_type = other RNA
nctgttttgcttttgtaactt

SEQ ID NO: 262 - RNA, 21 nt, synthetic construct
  source 1..21: mol_type = other RNA
nctgttttgcttttgtaactn

SEQ ID NO: 263 - RNA, 21 nt, synthetic construct
  source 1..21: mol_type = other RNA
nntgttttgcttttgtaactt

SEQ ID NO: 264 - RNA, 21 nt, synthetic construct
  source 1..21: mol_type = other RNA
cctgttttgcttttgtaacta

SEQ ID NO: 265 - RNA, 21 nt, synthetic construct
  source 1..21: mol_type = other RNA
nntgttttgcttttgtaactn

SEQ ID NO: 266 - RNA, 21 nt, synthetic construct
  source 1..21: mol_type = other RNA
nnngttttgcttttgtaactt

SEQ ID NO: 267 - RNA, 21 nt, synthetic construct
  source 1..21: mol_type = other RNA
nnngttttgcttttgtaacta

SEQ ID NO: 268 - RNA, 21 nt, synthetic construct
  source 1..21: mol_type = other RNA
nnngttttgcttttgtaactn

SEQ ID NO: 269 - RNA, 21 nt, synthetic construct
  source 1..21: mol_type = other RNA
ttttgcttttgtaacttgaag

SEQ ID NO: 270 - RNA, 21 nt, synthetic construct
  source 1..21: mol_type = other RNA
ttttgcttttgtaacttgaaa

SEQ ID NO: 271 - RNA, 21 nt, synthetic construct
  source 1..21: mol_type = other RNA
ttttgcttttgtaacttgaat

SEQ ID NO: 272 - RNA, 21 nt, synthetic construct
  source 1..21: mol_type = other RNA
ttttgcttttgtaacttgaan

SEQ ID NO: 273 - RNA, 21 nt, synthetic construct
  source 1..21: mol_type = other RNA
ntttgcttttgtaacttgaan

SEQ ID NO: 274 - RNA, 21 nt, synthetic construct
  source 1..21: mol_type = other RNA
ttttgtaacttgaagatattt

SEQ ID NO: 275 - RNA, 21 nt, synthetic construct
  source 1..21: mol_type = other RNA
ttttgtaacttgaagatatta

SEQ ID NO: 276 - RNA, 21 nt, synthetic construct
  source 1..21: mol_type = other RNA
ttttgtaacttgaagatattn

SEQ ID NO: 277 - RNA, 21 nt, synthetic construct
  source 1..21: mol_type = other RNA
ntttgtaacttgaagatattn

SEQ ID NO: 278 - RNA, 21 nt, synthetic construct
  source 1..21: mol_type = other RNA
tttgtaacttgaagatattta

SEQ ID NO: 279 - RNA, 21 nt, synthetic construct
  source 1..21: mol_type = other RNA
tttgtaacttgaagatatttt

SEQ ID NO: 280 - RNA, 21 nt, synthetic construct
  source 1..21: mol_type = other RNA
tttgtaacttgaagatatttn

SEQ ID NO: 281 - RNA, 21 nt, synthetic construct
  source 1..21: mol_type = other RNA
nttgtaacttgaagatatttn

SEQ ID NO: 282 - RNA, 21 nt, synthetic construct
  source 1..21: mol_type = other RNA
ttgtaacttgaagatatttat

SEQ ID NO: 283 - RNA, 21 nt, synthetic construct
  source 1..21: mol_type = other RNA
ttgtaacttgaagatatttaa

SEQ ID NO: 284 - RNA, 21 nt, synthetic construct
  source 1..21: mol_type = other RNA
ttgtaacttgaagatatttan

SEQ ID NO: 285 - RNA, 21 nt, synthetic construct
  source 1..21: mol_type = other RNA
ntgtaacttgaagatatttan

SEQ ID NO: 286 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluoroadenosine
  modified_base 5: mod_base = cm
  modified_base 6: mod_base = OTHER; note = 2'-fluoroadenosine
  modified_base 7: mod_base = um
  modified_base 8: mod_base = OTHER; note = 2'-fluoroguanosine
  modified_base 9: mod_base = cm
  modified_base 10: mod_base = OTHER; note = 2'-fluoroadenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = OTHER; note = 2'-fluoroguanosine
  modified_base 19: mod_base = gm
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
aagacatgcaggatcttggtg

SEQ ID NO: 287 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluorouridine
  modified_base 9: mod_base = gm
  modified_base 10: mod_base = OTHER; note = 2'-fluorouridine
  modified_base 11: mod_base = cm
  modified_base 12: mod_base = OTHER; note = 2'-fluorouridine
  modified_base 13: mod_base = gm
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = OTHER; note = 2'-fluorocytidine
  modified_base 19: mod_base = um
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
agataaatgtctgcttgcttg

SEQ ID NO: 288 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluorouridine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroguanosine
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-fluoroadenosine
  modified_base 19: mod_base = gm
  modified_base 20: mod_base = OTHER; note = 2'-fluoroadenosine
  modified_base 20^21: mod_base = OTHER; note = phosphorothioate linkage
  modified_base 21: mod_base = gm
  source 1..21: mol_type = other RNA
ttgtaaaaaggcaacagagag

SEQ ID NO: 289 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroguanosine
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = gm
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = OTHER; note = 2'-O-methyladenosine
  source 1..21: mol_type = other RNA
tctgtaaaaaggcaacagaga

SEQ ID NO: 290 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroadenosine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = um
  modified_base 18: mod_base = OTHER; note = 2'-fluorocytidine
  modified_base 19: mod_base = um
  modified_base 20: mod_base = OTHER; note = 2'-fluoroadenosine
  modified_base 20^21: mod_base = OTHER; note = phosphorothioate linkage
  modified_base 21: mod_base = gm
  source 1..21: mol_type = other RNA
acaaaagcaaaacaggtctag

SEQ ID NO: 291 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluoroguanosine
  modified_base 9: mod_base = cm
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-fluorouridine
  modified_base 19: mod_base = cm
  modified_base 20: mod_base = OTHER; note = 2'-fluorouridine
  modified_base 20^21: mod_base = OTHER; note = phosphorothioate linkage
  modified_base 21: mod_base = OTHER; note = 2'-O-methyladenosine
  source 1..21: mol_type = other RNA
tacaaaagcaaaacaggtcta

SEQ ID NO: 292 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluorouridine
  modified_base 5: mod_base = um
  modified_base 6: mod_base = OTHER; note = 2'-fluoroadenosine
  modified_base 7: mod_base = cm
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluorocytidine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 293 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluorouridine
  modified_base 5: mod_base = cm
  modified_base 6: mod_base = OTHER; note = 2'-fluorouridine
  modified_base 7: mod_base = um
  modified_base 8: mod_base = OTHER; note = 2'-fluoroguanosine
  modified_base 9: mod_base = gm
  modified_base 10: mod_base = OTHER; note = 2'-fluorouridine
  modified_base 11: mod_base = gm
  modified_base 12: mod_base = OTHER; note = 2'-fluoroadenosine
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluoroadenosine
  modified_base 17: mod_base = um
  modified_base 18: mod_base = OTHER; note = 2'-fluorocytidine
  modified_base 19: mod_base = cm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tgatcttggtgaggtatcccc

SEQ ID NO: 294 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluoroadenosine
  modified_base 5: mod_base = um
  modified_base 6: mod_base = OTHER; note = 2'-fluorocytidine
  modified_base 7: mod_base = um
  modified_base 8: mod_base = OTHER; note = 2'-fluorouridine
  modified_base 9: mod_base = gm
  modified_base 10: mod_base = OTHER; note = 2'-fluoroguanosine
  modified_base 11: mod_base = um
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluoroguanosine
  modified_base 15: mod_base = gm
  modified_base 16: mod_base = OTHER; note = 2'-fluorouridine
  modified_base 17: mod_base = OTHER; note = 2'-O-methyladenosine
  modified_base 18: mod_base = OTHER; note = 2'-fluorouridine
  modified_base 19: mod_base = cm
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
aggatcttggtgaggtatccc

SEQ ID NO: 295 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = cm
  modified_base 3^4: mod_base = OTHER; note = phosphorothioate linkage
  modified_base 4: mod_base = OTHER; note = 2'-fluoroadenosine
  modified_base 5: mod_base = um
  modified_base 6: mod_base = OTHER; note = 2'-fluoroguanosine
  modified_base 7: mod_base = OTHER; note = 2',3'-seco-cytidine
  modified_base 8: mod_base = OTHER; note = 2'-fluoroadenosine
  modified_base 9: mod_base = gm
  modified_base 10: mod_base = OTHER; note = 2'-fluoroguanosine
  modified_base 11: mod_base = OTHER; note = 2'-O-methyladenosine
  modified_base 12: mod_base = OTHER; note = 2'-fluorouridine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = gm
  modified_base 18: mod_base = OTHER; note = 2'-fluorouridine
  modified_base 19: mod_base = gm
  modified_base 20: mod_base = OTHER; note = 2'-fluoroadenosine
  modified_base 20^21: mod_base = OTHER; note = phosphorothioate linkage
  modified_base 21: mod_base = gm
  source 1..21: mol_type = other RNA
tacatgcaggatcttggtgag

SEQ ID NO: 296 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluorocytidine
  modified_base 5: mod_base = um
  modified_base 6: mod_base = OTHER; note = 2'-fluorouridine
  modified_base 7: mod_base = cm
  modified_base 8: mod_base = OTHER; note = 2'-fluorocytidine
  modified_base 9: mod_base = um
  modified_base 10: mod_base = OTHER; note = 2'-fluoroguanosine
  modified_base 11: mod_base = gm
  modified_base 12: mod_base = OTHER; note = 2'-fluorouridine
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = gm
  modified_base 18: mod_base = OTHER; note = 2'-fluorouridine
  modified_base 19: mod_base = um
  modified_base 20: mod_base = OTHER; note = 2'-fluorocytidine
  modified_base 20^21: mod_base = OTHER; note = phosphorothioate linkage
  modified_base 21: mod_base = cm
  source 1..21: mol_type = other RNA
tagcttcctggtctgtgttcc

SEQ ID NO: 297 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-fluorouridine
  modified_base 9: mod_base = cm
  modified_base 10: mod_base = OTHER; note = 2'-fluorouridine
  modified_base 11: mod_base = gm
  modified_base 12: mod_base = OTHER; note = 2'-fluorocytidine
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = OTHER; note = 2'-fluorouridine
  modified_base 19: mod_base = gm
  modified_base 20: mod_base = OTHER; note = 2'-fluoroguanosine
  modified_base 20^21: mod_base = OTHER; note = phosphorothioate linkage
  modified_base 21: mod_base = gm
  source 1..21: mol_type = other RNA
ataaatgtctgcttgcttggg

SEQ ID NO: 298 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-fluorocytidine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-O-methyladenosine
  modified_base 10: mod_base = OTHER; note = 2'-fluoroguanosine
  modified_base 11: mod_base = gm
  modified_base 12: mod_base = OTHER; note = 2'-fluoroadenosine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-fluorocytidine
  modified_base 19: mod_base = cm
  modified_base 20: mod_base = OTHER; note = 2'-fluoroadenosine
  modified_base 20^21: mod_base = OTHER; note = phosphorothioate linkage
  modified_base 21: mod_base = OTHER; note = 2'-O-methyladenosine
  source 1..21: mol_type = other RNA
taacagagaggacagacccaa

SEQ ID NO: 299 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluoroadenosine
  modified_base 9: mod_base = gm
  modified_base 10: mod_base = OTHER; note = 2'-fluoroguanosine
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-fluoroguanosine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = gm
  source 1..21: mol_type = other RNA
tgtaaaaaggcaacagagagg

SEQ ID NO: 300 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = cm
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroguanosine
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-fluoroguanosine
  modified_base 19: mod_base = gm
  modified_base 20: mod_base = OTHER; note = 2'-fluorouridine
  modified_base 20^21: mod_base = OTHER; note = phosphorothioate linkage
  modified_base 21: mod_base = cm
  source 1..21: mol_type = other RNA
tttacaaaagcaaaacaggtc

SEQ ID NO: 301 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = um
  modified_base 10: mod_base = OTHER; note = 2'-O-methyladenosine
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
tttcaagttacaaaagcaaac

SEQ ID NO: 302 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluorouridine
  modified_base 5: mod_base = um
  modified_base 6: mod_base = OTHER; note = 2'-fluoroadenosine
  modified_base 7: mod_base = cm
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluorocytidine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = cm
  source 1..21: mol_type = other RNA
aagttacaaaagcaaaacagc

SEQ ID NO: 303 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = OTHER; note = 2'-fluorouridine
  modified_base 5: mod_base = um
  modified_base 6: mod_base = OTHER; note = 2'-fluoroadenosine
  modified_base 7: mod_base = cm
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluorocytidine
  modified_base 19: mod_base = OTHER; note = 2'-O-methyladenosine
  modified_base 20: mod_base = OTHER; note = 2'-fluoroguanosine
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 304 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = OTHER; note = 2'-fluorouridine
  modified_base 5: mod_base = um
  modified_base 6: mod_base = OTHER; note = 2'-fluoroadenosine
  modified_base 7: mod_base = cm
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluorocytidine
  modified_base 19: mod_base = OTHER; note = 2'-O-methyladenosine
  modified_base 19^20: mod_base = OTHER; note = phosphorothioate linkage
  modified_base 20: mod_base = OTHER; note = 2'-fluoroguanosine
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 305 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 306 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-fluoroguanosine
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 307 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 308 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 309 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 310 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 311 - RNA, 21 nt, synthetic construct
  modified_base 1: mod_base = um
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tagttacaaaagcaaaacagg

SEQ ID NO: 312 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 3^4: mod_base = OTHER; note = phosphorothioate linkage
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tagttacaaaagcaaaacagg

SEQ ID NO: 313 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tagttacaaaagcaaaacagg

SEQ ID NO: 314 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tagttacaaaagcaaaacagg

SEQ ID NO: 315 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluoroadenosine
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tagttacaaaagcaaaacagg

SEQ ID NO: 316 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyl-2-aminoadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ttgtanaaaggcaacagagag

SEQ ID NO: 317 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 4: mod_base = cm
  modified_base 5: mod_base = gm
  modified_base 6: mod_base = cm
  modified_base 7: mod_base = OTHER; note = 2',3'-seco-uridine
  modified_base 8: mod_base = gm
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = um
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
agtcgctggaggcaccaatgg

SEQ ID NO: 318 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = um
  modified_base 11: mod_base = OTHER; note = 2'-O-methyladenosine
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = cm
  modified_base 18: mod_base = um
  modified_base 19: mod_base = gm
  modified_base 20: mod_base = cm
  modified_base 20^21: mod_base = OTHER; note = phosphorothioate linkage
  modified_base 21: mod_base = um
  source 1..21: mol_type = other RNA
tccaaaagataaatgtctgct

SEQ ID NO: 319 - DNA, 21 bp, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = gm
  modified_base 7: mod_base = gm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = cm
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other DNA
agagaggacagacccaaaagg

SEQ ID NO: 320 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = um
  modified_base 7: mod_base = cm
  modified_base 8: mod_base = um
  modified_base 9: mod_base = um
  modified_base 10: mod_base = cm
  modified_base 11: mod_base = OTHER; note = 2'-O-methyladenosine
  modified_base 12: mod_base = OTHER; note = 2'-fluoroadenosine
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
aaatatcttcaagttacaaag

SEQ ID NO: 321 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = um
  modified_base 8: mod_base = cm
  modified_base 9: mod_base = um
  modified_base 10: mod_base = um
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
taaatatcttcaagttacaag

SEQ ID NO: 322 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroguanosine
  modified_base 3: mod_base = um
  modified_base 4: mod_base = OTHER; note = 2'-O-methyladenosine
  modified_base 5: mod_base = gm
  modified_base 6: mod_base = cm
  modified_base 7: mod_base = OTHER; note = 2',3'-seco-adenosine
  modified_base 8: mod_base = gm
  modified_base 9: mod_base = gm
  modified_base 10: mod_base = cm
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = um
  modified_base 18: mod_base = gm
  modified_base 19: mod_base = gm
  modified_base 20: mod_base = cm
  modified_base 20^21: mod_base = OTHER; note = phosphorothioate linkage
  modified_base 21: mod_base = OTHER; note = 2'-O-methyladenosine
  source 1..21: mol_type = other RNA
tgtagcaggcagcacctggca

SEQ ID NO: 323 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = gm
  modified_base 8: mod_base = OTHER; note = 2'-O-methyladenosine
  modified_base 9: mod_base = cm
  modified_base 10: mod_base = cm
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = um
  modified_base 19: mod_base = OTHER; note = 2'-O-methyladenosine
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
aggacagacccaaaagataag

SEQ ID NO: 324 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-O-methyladenosine
  modified_base 5: mod_base = um
  modified_base 6: mod_base = gm
  modified_base 7: mod_base = um
  modified_base 8: mod_base = cm
  modified_base 9: mod_base = um
  modified_base 10: mod_base = gm
  modified_base 11: mod_base = cm
  modified_base 12: mod_base = OTHER; note = 2'-fluorouridine
  modified_base 13: mod_base = um
  modified_base 14: mod_base = OTHER; note = 2'-fluoroguanosine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = gm
  modified_base 20^21: mod_base = OTHER; note = phosphorothioate linkage
  modified_base 21: mod_base = um
  source 1..21: mol_type = other RNA
taaatgtctgcttgcttgggt

SEQ ID NO: 325 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorocytidine
  modified_base 2^3: mod_base = OTHER; note = phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = OTHER; note = 2',3'-seco-uridine
  modified_base 8: mod_base = cm
  modified_base 9: mod_base = gm
  modified_base 10: mod_base = gm
  modified_base 11: mod_base = um
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = um
  modified_base 18: mod_base = gm
  modified_base 19: mod_base = OTHER; note = 2'-O-methyladenosine
  modified_base 20: mod_base = cm
  modified_base 20^21: mod_base = OTHER; note = phosphorothioate linkage
  modified_base 21: mod_base = cm
  source 1..21: mol_type = other RNA
tcgaagtcggtgaccatgacc

SEQ ID NO: 326 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = gm
  modified_base 8: mod_base = gm
  modified_base 9: mod_base = um
  modified_base 10: mod_base = cm
  modified_base 11: mod_base = um
  modified_base 12: mod_base = OTHER; note = 2'-fluoroadenosine
  modified_base 13: mod_base = gm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = gm
  modified_base 19: mod_base = um
  modified_base 20: mod_base = um
  modified_base 20^21: mod_base = OTHER; note = phosphorothioate linkage
  modified_base 21: mod_base = gm
  source 1..21: mol_type = other RNA
aaaacaggtctagaaaagttg

SEQ ID NO: 327 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = um
  modified_base 10: mod_base = cm
  modified_base 11: mod_base = um
  modified_base 12: mod_base = OTHER; note = 2'-fluorouridine
  modified_base 13: mod_base = um
  modified_base 14: mod_base = OTHER; note = 2'-fluoroguanosine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = gm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
tgatgacatctttggcagagg

SEQ ID NO: 328 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = um
  modified_base 5: mod_base = cm
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-O-methyladenosine
  modified_base 10: mod_base = um
  modified_base 11: mod_base = OTHER; note = 2'-O-methyladenosine
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorocytidine
  modified_base 17: mod_base = cm
  modified_base 18: mod_base = OTHER; note = 2'-O-methyladenosine
  modified_base 19: mod_base = gm
  modified_base 20: mod_base = gm
  modified_base 20^21: mod_base = OTHER; note = phosphorothioate linkage
  modified_base 21: mod_base = cm
  source 1..21: mol_type = other RNA
ttttccgaataaactccaggc

SEQ ID NO: 329 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = um
  modified_base 9: mod_base = cm
  modified_base 10: mod_base = um
  modified_base 11: mod_base = um
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ataaatatcttcaagttacag

SEQ ID NO: 330 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ttgtaaaaaggcaacagagag

SEQ ID NO: 331 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluorouridine
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ttgtaaaaaggcaacagagag

SEQ ID NO: 332 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluorouridine
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyl-2-aminoadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ttgtanaaaggcaacagagag

SEQ ID NO: 333 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluorouridine
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = gm
  modified_base 12: mod_base = OTHER; note = 2'-fluorocytidine
  modified_base 13: mod_base = OTHER; note = 2'-O-methyladenosine
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = gm
  modified_base 18: mod_base = OTHER; note = 2'-O-methyladenosine
  modified_base 19: mod_base = gm
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
ttgtaaaaaggcaacagagag

SEQ ID NO: 334 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluoroadenosine
  modified_base 3: mod_base = OTHER; note = 2'-O-methyladenosine
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = um
  modified_base 8: mod_base = cm
  modified_base 9: mod_base = um
  modified_base 10: mod_base = um
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 20^21: mod_base = OTHER; note = phosphorothioate linkage
  modified_base 21: mod_base = gm
  source 1..21: mol_type = other RNA
taaatatcttcaagttacaag

SEQ ID NO: 335 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluoroadenosine
  modified_base 3: mod_base = OTHER; note = 2'-O-methyladenosine
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = um
  modified_base 8: mod_base = cm
  modified_base 9: mod_base = um
  modified_base 10: mod_base = um
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
taaatatcttcaagttacagg

SEQ ID NO: 336 - RNA, 21 nt, synthetic construct
  modified_base 1: mod_base = OTHER; note = 5'-cyclopropyl phosphonate-2'-O-methyluridine
  modified_base 1^2: mod_base = OTHER; note = phosphorothioate linkage
  modified_base 2: mod_base = OTHER; note = 2'-fluoroadenosine
  modified_base 3: mod_base = OTHER; note = 2'-O-methyladenosine
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = um
  modified_base 8: mod_base = cm
  modified_base 9: mod_base = um
  modified_base 10: mod_base = um
  modified_base 11: mod_base = cm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
taaatatcttcaagttacagg

SEQ ID NO: 337 - DNA, 21 bp, synthetic construct
  modified_base 1: mod_base = OTHER; note = 5'-cyclopropyl phosphonate-2'-O-methyladenosine
  modified_base 2: mod_base = OTHER; note = 2'-fluoroguanosine
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = gm
  modified_base 7: mod_base = gm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = cm
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other DNA
agagaggacagacccaaaagg

SEQ ID NO: 338 - RNA, 21 nt, synthetic construct
  modified_base 1: mod_base = OTHER; note = 5'-cyclopropyl phosphonate-2'-O-methyluridine
  modified_base 2: mod_base = OTHER; note = 2'-fluoroguanosine
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = gm
  modified_base 7: mod_base = gm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = cm
  modified_base 14: mod_base = OTHER; note = 2'-fluorocytidine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgagaggacagacccaaaagg

SEQ ID NO: 339 - RNA, 21 nt, synthetic construct
  modified_base 1: mod_base = OTHER; note = 5'-cyclopropyl phosphonate-2'-O-methyladenosine
  modified_base 2: mod_base = OTHER; note = 2'-fluoroadenosine
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = gm
  modified_base 8: mod_base = gm
  modified_base 9: mod_base = um
  modified_base 10: mod_base = cm
  modified_base 11: mod_base = um
  modified_base 12: mod_base = OTHER; note = 2'-fluoroadenosine
  modified_base 13: mod_base = gm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = gm
  modified_base 19: mod_base = um
  modified_base 20: mod_base = um
  modified_base 20^21: mod_base = OTHER; note = phosphorothioate linkage
  modified_base 21: mod_base = gm
  source 1..21: mol_type = other RNA
aaaacaggtctagaaaagttg

SEQ ID NO: 340 - RNA, 21 nt, synthetic construct
  modified_base 1: mod_base = OTHER; note = 5'-cyclopropyl phosphonate-2'-O-methyladenosine
  modified_base 2: mod_base = OTHER; note = 2'-fluoroadenosine
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 341 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 4: mod_base = um
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = um
  source 1..21: mol_type = other RNA
agttacaaaagcaaaacaggt

SEQ ID NO: 342 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = OTHER; note = 2'-fluorouridine
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 343 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-fluoroadenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 344 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 345 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 346 - RNA, 21 nt, synthetic construct
  modified_base 1: mod_base = OTHER; note = 5'-cyclopropyl phosphonate-2'-O-methyladenosine
  modified_base 1^2: mod_base = OTHER; note = phosphorothioate linkage
  modified_base 2: mod_base = OTHER; note = 2'-fluoroadenosine
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluoroguanosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluorocytidine
  modified_base 19: mod_base = OTHER; note = 2'-O-methyladenosine
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = gm
  source 1..21: mol_type = other RNA
aagttacaaaagcaaaacagg

SEQ ID NO: 347 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = um
  modified_base 13: mod_base = OTHER; note = 2'-fluorouridine
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtaactt

SEQ ID NO: 348 - RNA, 21 nt, synthetic construct
  modified_base 1: mod_base = cm
  modified_base 2: mod_base = OTHER; note = 2'-O-methyladenosine
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = gm
  modified_base 18: mod_base = um
  modified_base 19: mod_base = cm
  modified_base 20: mod_base = um
  modified_base 21: mod_base = um
  source 1..21: mol_type = other RNA
caccaagatcctgcatgtctt

SEQ ID NO: 349 - RNA, 21 nt, synthetic construct
  modified_base 1: mod_base = cm
  modified_base 2: mod_base = OTHER; note = 2'-O-methyl-2-aminoadenosine
  modified_base 3: mod_base = OTHER; note = 2'-O-methyladenosine
  modified_base 4: mod_base = gm
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroguanosine
  modified_base 12: mod_base = OTHER; note = 2'-O-methyladenosine
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = um
  source 1..21: mol_type = other RNA
cnagcaagcagacatttatct

SEQ ID NO: 350 - RNA, 21 nt, synthetic construct
  modified_base 1: mod_base = cm
  modified_base 2: mod_base = um
  modified_base 3: mod_base = cm
  modified_base 4: mod_base = um
  modified_base 5: mod_base = cm
  modified_base 6: mod_base = um
  modified_base 7: mod_base = gm
  modified_base 8: mod_base = um
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = cm
  modified_base 13: mod_base = um
  modified_base 14: mod_base = um
  modified_base 15: mod_base = um
  modified_base 16: mod_base = um
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methyladenosine
  source 1..21: mol_type = other RNA
ctctctgttgcctttttacaa

SEQ ID NO: 351 - RNA, 21 nt, synthetic construct
  modified_base 1: mod_base = um
  modified_base 2: mod_base = cm
  modified_base 3: mod_base = um
  modified_base 4: mod_base = cm
  modified_base 5: mod_base = um
  modified_base 6: mod_base = gm
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = um
  modified_base 14: mod_base = um
  modified_base 15: mod_base = um
  modified_base 16: mod_base = um
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = gm
  modified_base 21: mod_base = OTHER; note = 2'-O-methyladenosine
  source 1..21: mol_type = other RNA
tctctgttgcctttttacaga

SEQ ID NO: 352 - RNA, 21 nt, synthetic construct
  modified_base 1: mod_base = cm
  modified_base 2: mod_base = um
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = cm
  modified_base 7: mod_base = cm
  modified_base 8: mod_base = um
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorouridine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = um
  modified_base 19: mod_base = um
  modified_base 20: mod_base = gm
  modified_base 21: mod_base = um
  source 1..21: mol_type = other RNA
ctagacctgttttgcttttgt

SEQ ID NO: 353 - RNA, 21 nt, synthetic construct
  modified_base 1: mod_base = um
  modified_base 2: mod_base = OTHER; note = 2'-O-methyl-2-aminoadenosine
  modified_base 3: mod_base = gm
  modified_base 4: mod_base = OTHER; note = 2'-O-methyladenosine
  modified_base 5: mod_base = cm
  modified_base 6: mod_base = cm
  modified_base 7: mod_base = um
  modified_base 8: mod_base = gm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = um
  modified_base 18: mod_base = um
  modified_base 19: mod_base = gm
  modified_base 20: mod_base = um
  modified_base 21: mod_base = OTHER; note = 2'-O-methyladenosine
  source 1..21: mol_type = other RNA
tngacctgttttgcttttgta

SEQ ID NO: 354 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtaactt

SEQ ID NO: 355 - RNA, 21 nt, synthetic construct
  modified_base 1: mod_base = gm
  modified_base 2: mod_base = gm
  modified_base 3: mod_base = gm
  modified_base 4: mod_base = gm
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = cm
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-methyladenosine
  modified_base 13: mod_base = cm
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methylinosine
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
ggggatacctcaccaanatca

SEQ ID NO: 356 - RNA, 21 nt, synthetic construct
  modified_base 1: mod_base = gm
  modified_base 2: mod_base = gm
  modified_base 3: mod_base = gm
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = cm
  modified_base 8: mod_base = cm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = cm
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methylinosine
  modified_base 17: mod_base = OTHER; note = 2'-O-methyladenosine
  modified_base 18: mod_base = um
  modified_base 19: mod_base = cm
  modified_base 20: mod_base = cm
  modified_base 21: mod_base = um
  source 1..21: mol_type = other RNA
gggatacctcaccaanatcct

SEQ ID NO: 357 - RNA, 21 nt, synthetic construct
  modified_base 1: mod_base = cm
  modified_base 2: mod_base = um
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorouridine
  modified_base 12: mod_base = cm
  modified_base 13: mod_base = cm
  modified_base 14: mod_base = um
  modified_base 15: mod_base = gm
  modified_base 16: mod_base = cm
  modified_base 17: mod_base = OTHER; note = 2'-O-methyladenosine
  modified_base 18: mod_base = um
  modified_base 19: mod_base = gm
  modified_base 20: mod_base = um
  modified_base 21: mod_base = OTHER; note = 2'-O-methyladenosine
  source 1..21: mol_type = other RNA
ctcaccaagatcctgcatgta

SEQ ID NO: 358 - RNA, 21 nt, synthetic construct
  modified_base 1: mod_base = gm
  modified_base 2: mod_base = gm
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = gm
  modified_base 16: mod_base = OTHER; note = 2'-O-methyladenosine
  modified_base 17: mod_base = OTHER; note = 2'-O-methyladenosine
  modified_base 18: mod_base = OTHER; note = 2'-O-methylinosine
  modified_base 19: mod_base = cm
  modified_base 20: mod_base = um
  modified_base 21: mod_base = OTHER; note = 2'-O-methyladenosine
  source 1..21: mol_type = other RNA
ggaacacagaccaggaancta

SEQ ID NO: 359 - RNA, 21 nt, synthetic construct
  modified_base 1: mod_base = cm
  modified_base 2: mod_base = cm
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = cm
  modified_base 16: mod_base = OTHER; note = 2'-O-methyladenosine
  modified_base 17: mod_base = um
  modified_base 18: mod_base = um
  modified_base 19: mod_base = um
  modified_base 20: mod_base = OTHER; note = 2'-O-methyl-2-aminoadenosine
  modified_base 21: mod_base = um
  source 1..21: mol_type = other RNA
cccaagcaagcagacatttnt

SEQ ID NO: 360 - RNA, 21 nt, synthetic construct
  modified_base 1: mod_base = um
  modified_base 2: mod_base = um
  modified_base 3: mod_base = gm
  modified_base 4: mod_base = gm
  modified_base 5: mod_base = gm
  modified_base 6: mod_base = um
  modified_base 7: mod_base = cm
  modified_base 8: mod_base = um
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = cm
  modified_base 13: mod_base = um
  modified_base 14: mod_base = cm
  modified_base 15: mod_base = um
  modified_base 16: mod_base = cm
  modified_base 17: mod_base = um
  modified_base 18: mod_base = gm
  modified_base 19: mod_base = um
  modified_base 20: mod_base = um
  modified_base 21: mod_base = OTHER; note = 2'-O-methyladenosine
  source 1..21: mol_type = other RNA
ttgggtctgtcctctctgtta

SEQ ID NO: 361 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = um
  modified_base 6: mod_base = cm
  modified_base 7: mod_base = um
  modified_base 8: mod_base = gm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = cm
  modified_base 14: mod_base = um
  modified_base 15: mod_base = um
  modified_base 16: mod_base = um
  modified_base 17: mod_base = um
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine
  source 1..21: mol_type = other RNA
cctctctgttgcctttttaca

SEQ ID NO: 362 - RNA, 21 nt, synthetic construct
  modified_base 1: mod_base = gm
  modified_base 2: mod_base = OTHER; note = 2'-O-methyladenosine
  modified_base 3: mod_base = cm
  modified_base 4: mod_base = cm
  modified_base 5: mod_base = um
  modified_base 6: mod_base = gm
  modified_base 7: mod_base = um
  modified_base 8: mod_base = um
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = um
  modified_base 15: mod_base = um
  modified_base 16: mod_base = um
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyl-2-aminoadenosine
  modified_base 21: mod_base = OTHER; note = 2'-O-methyladenosine
  source 1..21: mol_type = other RNA
gacctgttttgcttttgtana

SEQ ID NO: 363 - RNA, 21 nt, synthetic construct
  modified_base 1: mod_base = gm
  modified_base 2: mod_base = um
  modified_base 3: mod_base = um
  modified_base 4: mod_base = um
  modified_base 5: mod_base = gm
  modified_base 6: mod_base = cm
  modified_base 7: mod_base = OTHER; note = 2'-fluorouridine
  modified_base 8: mod_base = um
  modified_base 9: mod_base = OTHER; note = 2'-fluorouridine
  modified_base 10: mod_base = um
  modified_base 11: mod_base = OTHER; note = 2'-fluoroguanosine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = um
  modified_base 18: mod_base = gm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
gtttgcttttgtaacttgaaa

SEQ ID NO: 364 - RNA, 21 nt, synthetic construct
  modified_base 1: mod_base = gm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
gctgttttgcttttgtaactt

SEQ ID NO: 365 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtnactt

SEQ ID NO: 366 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = OTHER; note = 2'-fluorouridine
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtaactt

SEQ ID NO: 367 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = OTHER; note = 2'-fluorouridine
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtaactt

SEQ ID NO: 368 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = um
  modified_base 13: mod_base = OTHER; note = 2'-fluorouridine
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtaactt

SEQ ID NO: 369 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = um
  modified_base 13: mod_base = OTHER; note = 2'-fluorouridine
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtnactt

SEQ ID NO: 370 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = OTHER; note = 2'-fluorouridine
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtnactt

SEQ ID NO: 371 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = um
  source 1..21: mol_type = other RNA
cctgttttgcttttgtnactt

SEQ ID NO: 372 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyladenosine
  source 1..21: mol_type = other RNA
cctgttttgcttttgtnacta

SEQ ID NO: 373 - RNA, 21 nt, synthetic construct
  modified_base 1: mod_base = cm
  modified_base 2: mod_base = um
  modified_base 3: mod_base = cm
  modified_base 4: mod_base = um
  modified_base 5: mod_base = cm
  modified_base 6: mod_base = um
  modified_base 7: mod_base = gm
  modified_base 8: mod_base = um
  modified_base 9: mod_base = OTHER; note = 2'-fluorouridine
  modified_base 10: mod_base = gm
  modified_base 11: mod_base = OTHER; note = 2'-fluorocytidine
  modified_base 12: mod_base = cm
  modified_base 13: mod_base = OTHER; note = 2'-fluorouridine
  modified_base 14: mod_base = um
  modified_base 15: mod_base = um
  modified_base 16: mod_base = um
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methyladenosine
  source 1..21: mol_type = other RNA
ctctctgttgcctttttacaa

SEQ ID NO: 374 - RNA, 21 nt, synthetic construct
  modified_base 1: mod_base = cm
  modified_base 2: mod_base = cm
  modified_base 3: mod_base = OTHER; note = 2'-O-methyladenosine
  modified_base 4: mod_base = um
  modified_base 5: mod_base = um
  modified_base 6: mod_base = gm
  modified_base 7: mod_base = gm
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorocytidine
  modified_base 12: mod_base = um
  modified_base 13: mod_base = OTHER; note = 2'-fluorocytidine
  modified_base 14: mod_base = cm
  modified_base 15: mod_base = OTHER; note = 2'-O-methyladenosine
  modified_base 16: mod_base = gm
  modified_base 17: mod_base = cm
  modified_base 18: mod_base = gm
  modified_base 19: mod_base = OTHER; note = 2'-O-methyladenosine
  modified_base 20: mod_base = cm
  modified_base 21: mod_base = um
  source 1..21: mol_type = other RNA
ccattggtgcctccagcgact

SEQ ID NO: 375 - RNA, 21 nt, synthetic construct
  modified_base 1: mod_base = OTHER; note = 2'-O-methyladenosine
  modified_base 2: mod_base = gm
  modified_base 3: mod_base = cm
  modified_base 4: mod_base = OTHER; note = 2'-O-methyladenosine
  modified_base 5: mod_base = gm
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorouridine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = um
  modified_base 17: mod_base = um
  modified_base 18: mod_base = um
  modified_base 19: mod_base = OTHER; note = 2'-O-methylinosine
  modified_base 20: mod_base = gm
  modified_base 21: mod_base = OTHER; note = 2'-O-methyladenosine
  source 1..21: mol_type = other RNA
agcagacatttatcttttnga

SEQ ID NO: 376 - RNA, 21 nt, synthetic construct
  modified_base 1: mod_base = cm
  modified_base 2: mod_base = cm
  modified_base 3: mod_base = um
  modified_base 4: mod_base = um
  modified_base 5: mod_base = um
  modified_base 6: mod_base = um
  modified_base 7: mod_base = gm
  modified_base 8: mod_base = gm
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = gm
  modified_base 14: mod_base = um
  modified_base 15: mod_base = cm
  modified_base 16: mod_base = cm
  modified_base 17: mod_base = um
  modified_base 18: mod_base = cm
  modified_base 19: mod_base = um
  modified_base 20: mod_base = cm
  modified_base 21: mod_base = um
  source 1..21: mol_type = other RNA
ccttttgggtctgtcctctct

SEQ ID NO: 377 - RNA, 21 nt, synthetic construct
  modified_base 1: mod_base = cm
  modified_base 2: mod_base = um
  modified_base 3: mod_base = um
  modified_base 4: mod_base = um
  modified_base 5: mod_base = gm
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorouridine
  modified_base 12: mod_base = gm
  modified_base 13: mod_base = OTHER; note = 2'-O-methyladenosine
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = um
  modified_base 20: mod_base = um
  modified_base 21: mod_base = um
  source 1..21: mol_type = other RNA
ctttgtaacttgaagatattt

SEQ ID NO: 378 - RNA, 21 nt, synthetic construct
  modified_base 1: mod_base = cm
  modified_base 2: mod_base = um
  modified_base 3: mod_base = um
  modified_base 4: mod_base = gm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = cm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = um
  modified_base 17: mod_base = OTHER; note = 2'-O-methyladenosine
  modified_base 18: mod_base = um
  modified_base 19: mod_base = um
  modified_base 20: mod_base = um
  modified_base 21: mod_base = OTHER; note = 2'-O-methyladenosine
  source 1..21: mol_type = other RNA
cttgtaacttgaagatattta

SEQ ID NO: 379 - RNA, 21 nt, synthetic construct
  modified_base 1: mod_base = um
  modified_base 2: mod_base = gm
  modified_base 3: mod_base = cm
  modified_base 4: mod_base = cm
  modified_base 5: mod_base = OTHER; note = 2'-O-methyladenosine
  modified_base 6: mod_base = gm
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = gm
  modified_base 13: mod_base = cm
  modified_base 14: mod_base = cm
  modified_base 15: mod_base = um
  modified_base 16: mod_base = gm
  modified_base 17: mod_base = cm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine
  source 1..21: mol_type = other RNA
tgccaggtgctgcctgctaca

SEQ ID NO: 380 - RNA, 21 nt, synthetic construct
  modified_base 1: mod_base = cm
  modified_base 2: mod_base = um
  modified_base 3: mod_base = um
  modified_base 4: mod_base = OTHER; note = 2'-O-methyladenosine
  modified_base 5: mod_base = um
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = gm
  modified_base 13: mod_base = gm
  modified_base 14: mod_base = um
  modified_base 15: mod_base = cm
  modified_base 16: mod_base = um
  modified_base 17: mod_base = OTHER; note = 2'-O-methylinosine
  modified_base 18: mod_base = um
  modified_base 19: mod_base = cm
  modified_base 20: mod_base = cm
  modified_base 21: mod_base = um
  source 1..21: mol_type = other RNA
cttatcttttgggtctntcct

SEQ ID NO: 381 - RNA, 21 nt, synthetic construct
  modified_base 1: mod_base = OTHER; note = 2'-O-methyladenosine
  modified_base 2: mod_base = cm
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroguanosine
  modified_base 12: mod_base = cm
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = cm
  modified_base 17: mod_base = OTHER; note = 2'-O-methyladenosine
  modified_base 18: mod_base = um
  modified_base 19: mod_base = um
  modified_base 20: mod_base = um
  modified_base 21: mod_base = OTHER; note = 2'-O-methyladenosine
  source 1..21: mol_type = other RNA
acccaagcaagcagacattta

SEQ ID NO: 382 - RNA, 21 nt, synthetic construct
  modified_base 1: mod_base = gm
  modified_base 2: mod_base = gm
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = gm
  modified_base 8: mod_base = gm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = cm
  modified_base 14: mod_base = gm
  modified_base 15: mod_base = OTHER; note = 2'-O-methyladenosine
  modified_base 16: mod_base = cm
  modified_base 17: mod_base = um
  modified_base 18: mod_base = um
  modified_base 19: mod_base = cm
  modified_base 20: mod_base = gm
  modified_base 21: mod_base = OTHER; note = 2'-O-methyladenosine
  source 1..21: mol_type = other RNA
ggtcatggtcaccgacttcga

SEQ ID NO: 383 - RNA, 21 nt, synthetic construct
  modified_base 1: mod_base = cm
  modified_base 2: mod_base = OTHER; note = 2'-O-methyladenosine
  modified_base 3: mod_base = OTHER; note = 2'-O-methyladenosine
  modified_base 4: mod_base = cm
  modified_base 5: mod_base = um
  modified_base 6: mod_base = um
  modified_base 7: mod_base = um
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = cm
  modified_base 15: mod_base = cm
  modified_base 16: mod_base = um
  modified_base 17: mod_base = gm
  modified_base 18: mod_base = um
  modified_base 19: mod_base = um
  modified_base 20: mod_base = um
  modified_base 21: mod_base = um
  source 1..21: mol_type = other RNA
caacttttctagacctgtttt

SEQ ID NO: 384 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = um
  modified_base 6: mod_base = gm
  modified_base 7: mod_base = cm
  modified_base 8: mod_base = cm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = um
  modified_base 15: mod_base = gm
  modified_base 16: mod_base = um
  modified_base 17: mod_base = cm
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctctgccaaagatgtcatca

SEQ ID NO: 385 - RNA, 21 nt, synthetic construct
  modified_base 1: mod_base = gm
  modified_base 2: mod_base = cm
  modified_base 3: mod_base = cm
  modified_base 4: mod_base = um
  modified_base 5: mod_base = gm
  modified_base 6: mod_base = gm
  modified_base 7: mod_base = OTHER; note = 2'-O-methyladenosine
  modified_base 8: mod_base = gm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluorouridine
  modified_base 12: mod_base = OTHER; note = 2'-O-methyladenosine
  modified_base 13: mod_base = um
  modified_base 14: mod_base = um
  modified_base 15: mod_base = cm
  modified_base 16: mod_base = gm
  modified_base 17: mod_base = gm
  modified_base 18: mod_base = OTHER; note = 2'-O-methyladenosine
  modified_base 19: mod_base = OTHER; note = 2'-O-methyladenosine
  modified_base 20: mod_base = OTHER; note = 2'-O-methyl-2-aminoadenosine
  modified_base 21: mod_base = OTHER; note = 2'-O-methyladenosine
  source 1..21: mol_type = other RNA
gcctggagtttattcggaana

SEQ ID NO: 386 - RNA, 21 nt, synthetic construct
  modified_base 1: mod_base = cm
  modified_base 2: mod_base = um
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = cm
  modified_base 8: mod_base = um
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = um
  modified_base 16: mod_base = OTHER; note = 2'-O-methyladenosine
  modified_base 17: mod_base = um
  modified_base 18: mod_base = um
  modified_base 19: mod_base = um
  modified_base 20: mod_base = OTHER; note = 2'-O-methyl-2-aminoadenosine
  modified_base 21: mod_base = um
  source 1..21: mol_type = other RNA
ctgtaacttgaagatatttnt

SEQ ID NO: 387 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = OTHER; note = 2'-fluorouridine
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyl-2-aminoadenosine
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyladenosine
  source 1..21: mol_type = other RNA
cctgttttgcttttgtnacta

SEQ ID NO: 388 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = cm
  modified_base 11: mod_base = OTHER; note = 2'-fluorouridine
  modified_base 12: mod_base = OTHER; note = 2'-fluorouridine
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyladenosine
  source 1..21: mol_type = other RNA
cctgttttgcttttgtaacta

SEQ ID NO: 389 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = um
  modified_base 7: mod_base = um
  modified_base 8: mod_base = um
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = um
  modified_base 15: mod_base = gm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyladenosine
  source 1..21: mol_type = other RNA
cctgttttgcttttgtaacta

SEQ ID NO: 390 - RNA, 21 nt, synthetic construct
  modified_base 1: mod_base = cm
  modified_base 2: mod_base = um
  modified_base 3: mod_base = cm
  modified_base 4: mod_base = um
  modified_base 5: mod_base = cm
  modified_base 6: mod_base = um
  modified_base 7: mod_base = gm
  modified_base 8: mod_base = um
  modified_base 9: mod_base = OTHER; note = 2'-fluorouridine
  modified_base 10: mod_base = gm
  modified_base 11: mod_base = OTHER; note = 2'-fluorocytidine
  modified_base 12: mod_base = OTHER; note = 2'-fluorocytidine
  modified_base 13: mod_base = um
  modified_base 14: mod_base = um
  modified_base 15: mod_base = um
  modified_base 16: mod_base = um
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methyladenosine
  source 1..21: mol_type = other RNA
ctctctgttgcctttttacaa

SEQ ID NO: 391 - RNA, 21 nt, synthetic construct
  modified_base 1: mod_base = cm
  modified_base 2: mod_base = um
  modified_base 3: mod_base = cm
  modified_base 4: mod_base = um
  modified_base 5: mod_base = cm
  modified_base 6: mod_base = um
  modified_base 7: mod_base = gm
  modified_base 8: mod_base = um
  modified_base 9: mod_base = um
  modified_base 10: mod_base = OTHER; note = 2'-fluoroguanosine
  modified_base 11: mod_base = OTHER; note = 2'-fluorocytidine
  modified_base 12: mod_base = cm
  modified_base 13: mod_base = um
  modified_base 14: mod_base = um
  modified_base 15: mod_base = um
  modified_base 16: mod_base = um
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methyladenosine
  source 1..21: mol_type = other RNA
ctctctgttgcctttttacaa

SEQ ID NO: 392 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = cm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = um
  modified_base 17: mod_base = OTHER; note = 2'-O-methyladenosine
  modified_base 18: mod_base = um
  modified_base 19: mod_base = um
  modified_base 20: mod_base = um
  modified_base 21: mod_base = OTHER; note = 2'-O-methyladenosine
  source 1..21: mol_type = other RNA
cctgtaacttgaagatattta

SEQ ID NO: 393 - RNA, 21 nt, synthetic construct
  modified_base 1: mod_base = cm
  modified_base 2: mod_base = cm
  modified_base 3: mod_base = um
  modified_base 4: mod_base = gm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = cm
  modified_base 9: mod_base = OTHER; note = 2'-fluorouridine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = um
  modified_base 17: mod_base = OTHER; note = 2'-O-methyl-2-aminoadenosine
  modified_base 18: mod_base = um
  modified_base 19: mod_base = um
  modified_base 20: mod_base = um
  modified_base 21: mod_base = OTHER; note = 2'-O-methyladenosine
  source 1..21: mol_type = other RNA
cctgtaacttgaagatnttta

SEQ ID NO: 394 - RNA, 21 nt, synthetic construct
  modified_base 1: mod_base = cm
  modified_base 2: mod_base = cm
  modified_base 3: mod_base = um
  modified_base 4: mod_base = um
  modified_base 5: mod_base = um
  modified_base 6: mod_base = um
  modified_base 7: mod_base = gm
  modified_base 8: mod_base = gm
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = gm
  modified_base 14: mod_base = um
  modified_base 15: mod_base = cm
  modified_base 16: mod_base = cm
  modified_base 17: mod_base = um
  modified_base 18: mod_base = cm
  modified_base 19: mod_base = um
  modified_base 20: mod_base = cm
  modified_base 21: mod_base = OTHER; note = 2'-O-methyladenosine
  source 1..21: mol_type = other RNA
ccttttgggtctgtcctctca

SEQ ID NO: 395 - RNA, 21 nt, synthetic construct
  source 1..21: mol_type = other RNA
aagacatgcaggatcttggtg

SEQ ID NO: 396 - RNA, 21 nt, synthetic construct
  source 1..21: mol_type = other RNA
agataaatgtctgcttgcttg

SEQ ID NO: 397 - RNA, 21 nt, synthetic construct
  source 1..21: mol_type = other RNA
ttgtaaaaaggcaacagagag

SEQ ID NO: 398 - RNA, 21 nt, synthetic construct
  source 1..21: mol_type = other RNA
tctgtaaaaaggcaacagaga

SEQ ID NO: 399 - RNA, 21 nt, synthetic construct
  source 1..21: mol_type = other RNA
acaaaagcaaaacaggtctag

SEQ ID NO: 400 - RNA, 21 nt, synthetic construct
  source 1..21: mol_type = other RNA
tacaaaagcaaaacaggtcta

SEQ ID NO: 401 - RNA, 21 nt, synthetic construct
  source 1..21: mol_type = other RNA
aagttacaaaagcaaaacagg

SEQ ID NO: 402 - RNA, 21 nt, synthetic construct
  source 1..21: mol_type = other RNA
tgatcttggtgaggtatcccc

SEQ ID NO: 403 - RNA, 21 nt, synthetic construct
  source 1..21: mol_type = other RNA
aggatcttggtgaggtatccc

SEQ ID NO: 404 - RNA, 21 nt, synthetic construct
  source 1..21: mol_type = other RNA
tacatgcaggatcttggtgag

SEQ ID NO: 405 - RNA, 21 nt, synthetic construct
  source 1..21: mol_type = other RNA
tagcttcctggtctgtgttcc

SEQ ID NO: 406 - RNA, 21 nt, synthetic construct
  source 1..21: mol_type = other RNA
ataaatgtctgcttgcttggg

SEQ ID NO: 407 - RNA, 21 nt, synthetic construct
  source 1..21: mol_type = other RNA
taacagagaggacagacccaa

SEQ ID NO: 408 - RNA, 21 nt, synthetic construct
  source 1..21: mol_type = other RNA
tgtaaaaaggcaacagagagg

SEQ ID NO: 409 - RNA, 21 nt, synthetic construct
  source 1..21: mol_type = other RNA
tttacaaaagcaaaacaggtc

SEQ ID NO: 410 - RNA, 21 nt, synthetic construct
  source 1..21: mol_type = other RNA
tttcaagttacaaaagcaaac

SEQ ID NO: 411 - RNA, 21 nt, synthetic construct
  source 1..21: mol_type = other RNA
aagttacaaaagcaaaacagc

SEQ ID NO: 412 - RNA, 21 nt, synthetic construct
  source 1..21: mol_type = other RNA
tagttacaaaagcaaaacagg

SEQ ID NO: 413 - RNA, 21 nt, synthetic construct
  modified_base 6: mod_base = OTHER; note = 2-aminoadenosine nucleotide
  source 1..21: mol_type = other RNA
ttgtanaaaggcaacagagag

SEQ ID NO: 414 - RNA, 21 nt, synthetic construct
  source 1..21: mol_type = other RNA
agtcgctggaggcaccaatgg

SEQ ID NO: 415 - RNA, 21 nt, synthetic construct
  source 1..21: mol_type = other RNA
tccaaaagataaatgtctgct

SEQ ID NO: 416 - DNA, 21 bp, synthetic construct
  source 1..21: mol_type = other DNA
agagaggacagacccaaaagg

SEQ ID NO: 417 - RNA, 21 nt, synthetic construct
  source 1..21: mol_type = other RNA
aaatatcttcaagttacaaag

SEQ ID NO: 418 - RNA, 21 nt, synthetic construct
  source 1..21: mol_type = other RNA
taaatatcttcaagttacaag

SEQ ID NO: 419 - RNA, 21 nt, synthetic construct
  source 1..21: mol_type = other RNA
tgtagcaggcagcacctggca

SEQ ID NO: 420 - RNA, 21 nt, synthetic construct
  source 1..21: mol_type = other RNA
aggacagacccaaaagataag

SEQ ID NO: 421 - RNA, 21 nt, synthetic construct
  source 1..21: mol_type = other RNA
taaatgtctgcttgcttgggt

SEQ ID NO: 422 - RNA, 21 nt, synthetic construct
  source 1..21: mol_type = other RNA
tcgaagtcggtgaccatgacc

SEQ ID NO: 423 - RNA, 21 nt, synthetic construct
  source 1..21: mol_type = other RNA
aaaacaggtctagaaaagttg

SEQ ID NO: 424 - RNA, 21 nt, synthetic construct
  source 1..21: mol_type = other RNA
tgatgacatctttggcagagg

SEQ ID NO: 425 - RNA, 21 nt, synthetic construct
  source 1..21: mol_type = other RNA
ttttccgaataaactccaggc

SEQ ID NO: 426 - RNA, 21 nt, synthetic construct
  source 1..21: mol_type = other RNA
ataaatatcttcaagttacag

SEQ ID NO: 427 - RNA, 21 nt, synthetic construct
  source 1..21: mol_type = other RNA
taaatatcttcaagttacagg

SEQ ID NO: 428 - RNA, 21 nt, synthetic construct
  source 1..21: mol_type = other RNA
tgagaggacagacccaaaagg

SEQ ID NO: 429 - RNA, 21 nt, synthetic construct
  source 1..21: mol_type = other RNA
agttacaaaagcaaaacaggt

SEQ ID NO: 430 - RNA, 21 nt, synthetic construct
  source 1..21: mol_type = other RNA
cctgttttgcttttgtaactt

SEQ ID NO: 431 - RNA, 21 nt, synthetic construct
  source 1..21: mol_type = other RNA
caccaagatcctgcatgtctt

SEQ ID NO: 432 - RNA, 21 nt, synthetic construct
  modified_base 2: mod_base = OTHER; note = 2-aminoadenosine nucleotide
  source 1..21: mol_type = other RNA
cnagcaagcagacatttatct

SEQ ID NO: 433 - RNA, 21 nt, synthetic construct
  source 1..21: mol_type = other RNA
ctctctgttgcctttttacaa

SEQ ID NO: 434 - RNA, 21 nt, synthetic construct
  source 1..21: mol_type = other RNA
tctctgttgcctttttacaga

SEQ ID NO: 435 - RNA, 21 nt, synthetic construct
  source 1..21: mol_type = other RNA
ctagacctgttttgcttttgt

SEQ ID NO: 436 - RNA, 21 nt, synthetic construct
  modified_base 2: mod_base = OTHER; note = 2-aminoadenosine nucleotide
  source 1..21: mol_type = other RNA
tngacctgttttgcttttgta

SEQ ID NO: 437 - RNA, 21 nt, synthetic construct
  modified_base 17: mod_base = i
  source 1..21: mol_type = other RNA
ggggatacctcaccaanatca

SEQ ID NO: 438 - RNA, 21 nt, synthetic construct
  modified_base 16: mod_base = i
  source 1..21: mol_type = other RNA
gggatacctcaccaanatcct

SEQ ID NO: 439 - RNA, 21 nt, synthetic construct
  source 1..21: mol_type = other RNA
ctcaccaagatcctgcatgta

SEQ ID NO: 440 - RNA, 21 nt, synthetic construct
  modified_base 18: mod_base = i
  source 1..21: mol_type = other RNA
ggaacacagaccaggaancta

SEQ ID NO: 441 - RNA, 21 nt, synthetic construct
  modified_base 20: mod_base = OTHER; note = 2-aminoadenosine nucleotide
  source 1..21: mol_type = other RNA
cccaagcaagcagacatttnt

SEQ ID NO: 442 - RNA, 21 nt, synthetic construct
  source 1..21: mol_type = other RNA
ttgggtctgtcctctctgtta

SEQ ID NO: 443 - RNA, 21 nt, synthetic construct
  source 1..21: mol_type = other RNA
cctctctgttgcctttttaca

SEQ ID NO: 444 - RNA, 21 nt, synthetic construct
  modified_base 20: mod_base = OTHER; note = 2-aminoadenosine nucleotide
  source 1..21: mol_type = other RNA
gacctgttttgcttttgtana

SEQ ID NO: 445 - RNA, 21 nt, synthetic construct
  source 1..21: mol_type = other RNA
gtttgcttttgtaacttgaaa

SEQ ID NO: 446 - RNA, 21 nt, synthetic construct
  source 1..21: mol_type = other RNA
gctgttttgcttttgtaactt

SEQ ID NO: 447 - RNA, 21 nt, synthetic construct
  modified_base 17: mod_base = OTHER; note = 2-aminoadenosine nucleotide
  source 1..21: mol_type = other RNA
cctgttttgcttttgtnactt

SEQ ID NO: 448 - RNA, 21 nt, synthetic construct
  modified_base 17: mod_base = OTHER; note = 2-aminoadenosine nucleotide
  source 1..21: mol_type = other RNA
cctgttttgcttttgtnacta

SEQ ID NO: 449 - RNA, 21 nt, synthetic construct
  source 1..21: mol_type = other RNA
ccattggtgcctccagcgact

SEQ ID NO: 450 - RNA, 21 nt, synthetic construct
  modified_base 19: mod_base = i
  source 1..21: mol_type = other RNA
agcagacatttatcttttnga

SEQ ID NO: 451 - RNA, 21 nt, synthetic construct
  source 1..21: mol_type = other RNA
ccttttgggtctgtcctctct

SEQ ID NO: 452 - RNA, 21 nt, synthetic construct
  source 1..21: mol_type = other RNA
ctttgtaacttgaagatattt

SEQ ID NO: 453 - RNA, 21 nt, synthetic construct
  source 1..21: mol_type = other RNA
cttgtaacttgaagatattta

SEQ ID NO: 454 - RNA, 21 nt, synthetic construct
  source 1..21: mol_type = other RNA
tgccaggtgctgcctgctaca

SEQ ID NO: 455 - RNA, 21 nt, synthetic construct
  modified_base 17: mod_base = i
  source 1..21: mol_type = other RNA
cttatcttttgggtctntcct

SEQ ID NO: 456 - RNA, 21 nt, synthetic construct
  source 1..21: mol_type = other RNA
acccaagcaagcagacattta

SEQ ID NO: 457 - RNA, 21 nt, synthetic construct
  source 1..21: mol_type = other RNA
ggtcatggtcaccgacttcga

SEQ ID NO: 458 - RNA, 21 nt, synthetic construct
  source 1..21: mol_type = other RNA
caacttttctagacctgtttt

SEQ ID NO: 459 - RNA, 21 nt, synthetic construct
  source 1..21: mol_type = other RNA
cctctgccaaagatgtcatca

SEQ ID NO: 460 - RNA, 21 nt, synthetic construct
  modified_base 20: mod_base = OTHER; note = 2-aminoadenosine nucleotide
  source 1..21: mol_type = other RNA
gcctggagtttattcggaana

SEQ ID NO: 461 - RNA, 21 nt, synthetic construct
  modified_base 20: mod_base = OTHER; note = 2-aminoadenosine nucleotide
  source 1..21: mol_type = other RNA
ctgtaacttgaagatatttnt

SEQ ID NO: 462 - RNA, 21 nt, synthetic construct
  source 1..21: mol_type = other RNA
cctgttttgcttttgtaacta

SEQ ID NO: 463 - RNA, 21 nt, synthetic construct
  source 1..21: mol_type = other RNA
cctgtaacttgaagatattta

SEQ ID NO: 464 - RNA, 21 nt, synthetic construct
  modified_base 17: mod_base = OTHER; note = 2-aminoadenosine nucleotide
  source 1..21: mol_type = other RNA
cctgtaacttgaagatnttta

SEQ ID NO: 465 - RNA, 21 nt, synthetic construct
  source 1..21: mol_type = other RNA
ccttttgggtctgtcctctca

SEQ ID NO: 466 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 4: mod_base = um
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = um
  source 1..21: mol_type = other RNA
agttacaaaagcaaaacaggt

SEQ ID NO: 467 - RNA, 19 nt, synthetic construct
  modified_base 1: mod_base = cm
  modified_base 1^2: mod_base = OTHER; note = phosphorothioate linkage
  modified_base 2: mod_base = um
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = um
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluorocytidine
  modified_base 10: mod_base = um
  modified_base 11: mod_base = um
  modified_base 12: mod_base = um
  modified_base 13: mod_base = um
  modified_base 14: mod_base = gm
  modified_base 15: mod_base = um
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = um
  source 1..19: mol_type = other RNA
ctgttttgcttttgtaact

SEQ ID NO: 468 - RNA, 21 nt, synthetic construct
  modified_base 1: mod_base = OTHER; note = 2'-O-methyladenosine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = um
  modified_base 4: mod_base = um
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = gm
  modified_base 19^20: mod_base = OTHER; note = phosphorothioate linkage
  modified_base 20: mod_base = gm
  modified_base 20^21: mod_base = OTHER; note = phosphorothioate linkage
  modified_base 21: mod_base = um
  source 1..21: mol_type = other RNA
agttacaaaagcaaaacaggt

SEQ ID NO: 469 - RNA, 19 nt, synthetic construct
  modified_base 1: mod_base = cm
  modified_base 1^2: mod_base = OTHER; note = phosphorothioate linkage
  modified_base 2: mod_base = um
  modified_base 2^3: mod_base = OTHER; note = phosphorothioate linkage
  modified_base 3: mod_base = gm
  modified_base 4: mod_base = um
  modified_base 5: mod_base = um
  modified_base 6: mod_base = um
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluorocytidine
  modified_base 10: mod_base = um
  modified_base 11: mod_base = um
  modified_base 12: mod_base = um
  modified_base 13: mod_base = um
  modified_base 14: mod_base = gm
  modified_base 15: mod_base = um
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = um
  source 1..19: mol_type = other RNA
ctgttttgcttttgtaact

(19) United States
(12) Patent Application Publication
(10) Pub. No.: US 2025/0376688 A1
(43) Pub. Date: Dec. 11, 2025

(54) RNAi Agents for Inhibiting Expression of Proprotein Convertase Subtilisin Kexin 9 (PCSK9), Pharmaceutical Compositions Thereof, and Methods of Use

(71) Applicant: Arrowhead Pharmaceuticals, Inc., Pasadena, CA (US)

(72) Inventors: Zhao Xu, San Diego, CA (US); Agnieszka Glebocka, Madison, WI (US); Casi Schienebeck, Deerfield, WI (US); Anthony Nicholas, Oregon, WI (US); Tao Pei, Middleton, WI (US)

(21) Appl. No.: 19/311,242

(22) Filed: Aug. 27, 2025

Related U.S. Application Data
(63) Continuation of application No. PCT/US24/17797, filed on Feb. 29, 2024.
(60) Provisional application No. 63/487,899, filed on Mar. 2, 2023.

(51) Int. Cl. C12N 15/113; A61K 47/02; A61P 3/06

(52) U.S. Cl. CPC C12N 15/1137; A61K 47/02; A61P 3/06; C12N 2310/11; C12N 2310/14; C12N 2310/315; C12N 2310/321; C12N 2310/322; C12N 2310/351; C12Y 304/21061

(57) ABSTRACT

The present disclosure relates to RNAi agents, e.g., double stranded RNAi agents such as small interfering RNA (siRNA) molecules, able to inhibit proprotein convertase subtilisin kexin 9 (PCSK9) gene expression. Also disclosed are pharmaceutical compositions that include PCSK9 RNAi agents and methods of use thereof. The PCSK9 RNAi agents disclosed herein may be conjugated to targeting ligands, including ligands that comprise N-acetyl-galactosamine, to facilitate the delivery to hepatocyte cells. Delivery of the PCSK9 RNAi agents in vivo provides for in vivo provides for inhibition of PCSK9 gene expression and thereby reduction of PCSK9 protein. The RNAi agents can be used in methods of treatment of diseases or disorders mediated at least in part by PCSK9 gene expression, including among others hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD).

### CROSS REFERENCE TO RELATED APPLICATIONS

This application is a continuation application of International Application No. PCT/US2024/017797, filed on Feb. 29, 2024, which claims the benefit of priority to U.S. Provisional Patent Application Ser. No. 63/487,899, filed on 2 Mar. 2023, the contents of which are incorporated herein by reference in their entirety.

### SEQUENCE LISTING

This application contains a Sequence Listing (in compliance with Standard ST26), which has been submitted in xml format and is hereby incorporated by reference in its entirety. The xml sequence listing file is names 30708-US1_SeqListing.xnl, created Aug. 26, 2025, and is 1,618,138 bytes in size.

### FIELD OF THE INVENTION

The present disclosure relates to RNA interference (RNAi) agents, e.g., double stranded RNAi agents, for inhibition proprotein convertase subtilisin kexin 9 (PCSK9) gene expression, pharmaceutical compositions that include PCSK9 RNAi agents, and methods of use thereof for the treatment of PCSK9-related diseases and disorders.

### BACKGROUND

Proprotein convertase subtilisin kexin 9, or PCSK9, is a key player in plasma cholesterol metabolism and is identified by scientists in both academia and industry as a target for treating hypercholesterolemia (Peterson et al., J Lipid Res. 2008). Low density lipoprotein (LDL) is the major transporter of cholesterol in the bloodstream. LDL-cholesterol (LDL-C) is normally removed from the bloodstream through receptor-mediated endocytosis in the liver via the LDL receptor (Horton et al., 2006). Maintaining adequate levels of the LDL receptor is critical to prevent buildup of LDL-C, as an excessively high level of LDL-C in blood (hypercholesterolemia) and tissues is associated with development of atherosclerotic plaques and cardiovascular disease (Horton et al., 2006; Klein-Szanto & Bassi, 2019). Mutations in the LDL receptor or mutations that impact binding of LDL to the receptor have been demonstrated to cause hypercholesterolemia. In addition, gain-of-function mutations in PCSK9 have been found to increase LDL levels by promoting degradation of the LDL receptor, and loss-of-function mutations result in hypocholesterolemia and significantly lower risk of coronary heart disease (Horton et al., 2006; Klein-Szanto & Bassi, 2019).

Accordingly, PCSK9 has become a therapeutic target for cholesterol-lowering therapy. Statins that increase expression of the LDL receptor and antibody treatments that inhibit PCSK9 are already on the market, but some patients experience tolerability issues that preclude their use as an effective treatment. Various small molecule inhibitors of PCSK9, as well as inhibitors that block LDL receptor binding, are in clinical development, and a small interfering RNA (siRNA) specific to PCSK9 mRNA has recently received approval in the UK and US, but yet these too all have certain shortcomings. Given the role of PCSK9 in controlling plasma LDL levels and the severity of disease if hypercholesterolemia remains uncontrolled, further development of additional inhibitory therapeutics could provide needed options to improve the cardiovascular health of patients with elevated LDL-C.

### SUMMARY

Disclosed herein are novel RNAi agents for inhibiting expression of a PCSK9 gene.

In some embodiments, the PCSK9 RNAi agents comprise:
an antisense strand wherein nucleotides 1-21 of the antisense strand comprise nucleotides 1-21 of the antisense strand sequences of Table 2, and
a sense strand comprising a nucleotide sequence that is at least partially complementary to the antisense strand,
wherein all or substantially all of the nucleotides of the antisense strand and/or the sense strand are modified nucleotides, and wherein the RNAi agent is linked to a targeting ligand that comprises N-acetyl-galactosamine.

In some embodiments, the sense strand comprises a nucleotide sequence of at least 15 contiguous nucleotides differing by 0 or 1 nucleotides from 15 contiguous nucleotides of any one of the sense strand sequences of Table 2, Table 4, Table 5C, Table 7B, or Table 8, and wherein the sense strand has a region of at least 85% complementarity over the 15 contiguous nucleotides to the antisense strand.

In some embodiments, at least one nucleotide of the RNAi agent includes a modified intemucleoside linkage.

In some embodiments, the modified nucleotides of the PCSK9 RNAi agents disclosed herein are selected from the group consisting of. 2′-O-methyl nucleotide, 2′-fluoro nucleotide (also referred to as a 2′-deoxy-2′-fluoro nucleotide), 2′-deoxy nucleotide, 2′,3′-seco nucleotide mimic (also referred to as an unlocked nucleotide or a UNA), locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, 5′-vinylphosphonate-containing nucleotide, 5′-cyclopropylphosphonate-containing nucleotide, and 3′-O-methyl nucleotide.

In other embodiments, all or substantially all of the modified nucleotides of the RNAi agents disclosed herein are 2′-O-methyl nucleotides, 2′-fluoro nucleotides, or combinations thereof.

In some embodiments, the 5′ end of the antisense strand includes a 5′-cyclopropylphosphonate-2′-O-methyl nucleotide.

In some embodiments, the antisense strand consists of, consists essentially of, or comprises the nucleotide sequence of any one of the modified antisense strand sequences of Table 3.

In some embodiments, the sense strand consists of, consists essentially of, or comprises the nucleotide sequence of any of the modified sense strand sequences of Table 4.

In some embodiments, the antisense strand comprises the nucleotide sequence of any one of the modified sequences of Table 3 and the sense strand comprises the nucleotide sequence of any one of the modified sequences of Table 4.

The RNAi agents disclosed herein are linked to a targeting ligand that comprises N-acetyl-galactosamine. In further embodiments, the targeting ligand is linked to the sense strand. In some embodiments, the targeting ligand is linked to the 5′ terminal end of the sense strand.

In some embodiments, the sense strand is between 15 and 30 nucleotides in length, and the antisense strand is between 21 and 30 nucleotides in length. In other embodiments, the sense strand and the antisense strand are each between 21 and 27 nucleotides in length. In other embodiments, the sense strand and the antisense strand are each between 21 and 24 nucleotides in length. In still other embodiments, sense strand and the antisense strand are each 21 nucleotides in length.

In some embodiments, the RNAi agents have two blunt ends.

In some embodiments, the sense strand comprises one or two terminal caps. In other embodiments, the sense strand comprises one or two inverted abasic residues.

In some embodiments, the RNAi agents are comprised of a sense strand and an antisense strand that form a duplex sequence of the duplex structures shown in Table 5A, 5B, 5C, or 8.

In some embodiments, the sense strand further includes inverted abasic residues at the 3′ terminal end of the nucleotide sequence, at the 5′ end of the nucleotide sequence, or at both.

In some embodiments, the targeting ligand comprises:

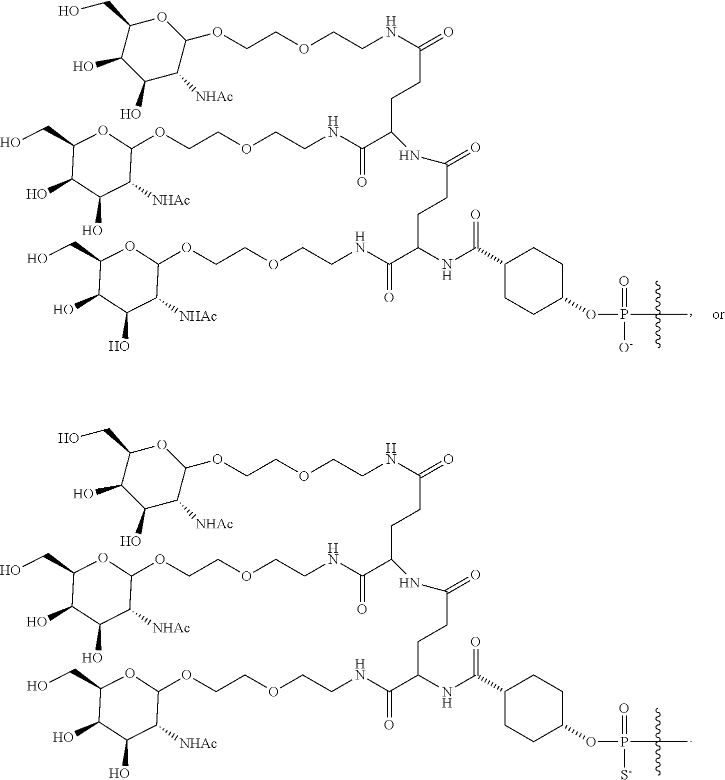

In further embodiments, the targeting ligand is linked to the sense strand of an RNAi agent by a phosphodiester or phosphorothioate linkage, and comprises or consists of:

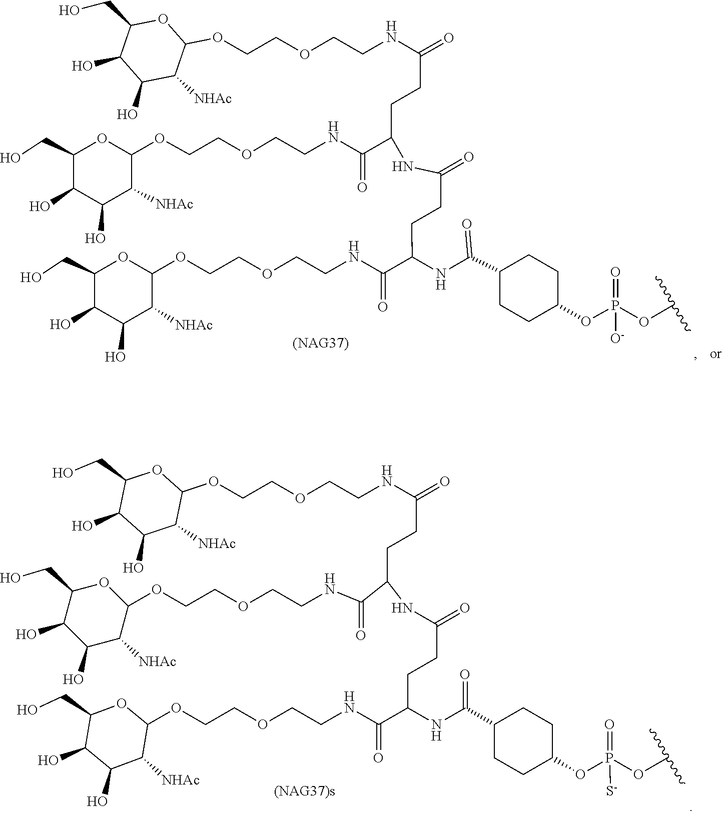

In some embodiment, the PCSK9 RNAi agent comprises an antisense strand comprising or consisting of the nucleotide sequence of SEQ ID NO:401 and a sense strand comprising or consisting of the nucleotide sequence of SEQ ID NO:4301

In some embodiment, the PCSK9 RNAi agent comprises a modified antisense strand comprising or consisting of the nucleotide sequence of SEQ ID NO:309 and a modified sense strand comprising or consisting of the nucleotide sequence of SEQ ID NO:368.

Also disclosed herein are compositions comprising the disclosed RNAi agents, wherein the compositions further comprise a pharmaceutically acceptable excipient.

Additionally, provided herein are methods for inhibiting expression of a PCSK9 gene in a hepatocyte cell in a human subject in vivo, the methods comprising introducing into the subject an effective amount of the disclosed PCSK9 RNAi agents or the disclosed compositions.

Further provided herein are methods of treating a PCSK9-related disease, disorder, or symptom, the methods comprising administering to a human subject in need thereof a therapeutically effective amount of the disclosed compositions.

In some embodiments, the disease is including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease.

In some embodiments, the RNAi agents are administered at a dose of about 0.05 mg/kg to about 5.0 mg/kg of body weight of the human subject. In some embodiments, the PCSK9 RNAi agents disclosed herein are administered in a fixed dose of a single injection containing about 25 mg, about 50 mg, about 100 mg, about 200 mg, about 300 mg, or about 400 mg of PCSK9 RNAi Agent Drug Substance, as described in Table 8.

Also provided herein are usages of the disclosed RNAi agents or the disclosed compositions, for the treatment of a disease, disorder, or symptom that is mediated at least in part by PCSK9 gene expression.

Further provided herein are usages of the disclosed RNAi agents or the disclosed compositions, for the preparation of a pharmaceutical compositions for treating a disease, disorder, or symptom that is mediated at least in part by PCSK9 gene expression.

### BRIEF DESCRIPTION OF THE DRAWINGS

FIG. 1A-1D. Chemical structure representation of PCSK9 RNAi agent AD09327 with the targeting ligand (NAG37)s linked to the 5′ end of the sense strand (SEQ ID PAIR NOs: 309/368), shown as a free acid.

FIG. 2A-2D. Chemical structure representation of PCSK9 RNAi agent AD09327 with the targeting ligand (NAG37)s linked to the 5′ end of the sense strand (SEQ ID PAIR NOs: 309/368), shown as a sodium salt.

### DETAILED DESCRIPTION

The disclosed RNAi agents, compositions thereof, and methods of use may be understood more readily by reference to the following detailed description, which form a part of this disclosure. It is to be understood that the disclosure is not limited to what is specifically described and/or shown herein, and that the terminology used herein is for the purpose of describing particular embodiments by way of example only and is not intended to be limiting.

It is to be appreciated that while certain features of the disclosures included herein are, for clarity, described herein in the context of separate embodiments, they may also be provided in combination in a single embodiment. Conversely, various features of the disclosed methods that are, for brevity, described in the context of a single embodiment, may also be provided separately or in any subcombination.

### Definitions

As used herein, an “RNAi agent” means a chemical composition of matter that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence-specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e., PCSK9 mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature. A nucleotide sequence can comprise unmodified and/or modified nucleotides. An oligonucleotide or nucleic acid molecule can comprise unmodified and/or modified nucleotides.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “nucleotide” has the same meaning as commonly understood in the art. Thus, the term “nucleotide” as used herein, refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as nucleotide analogs herein. Herein, a single nucleotide can be referred to as a monomer or unit.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a PCSK9 mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “individual”, “patient” and “subject”, are used interchangeably to refer to a member of any animal species including, but not limited to, birds, humans and other primates, and other mammals including commercially relevant mammals or animal models such as mice, rats, monkeys, cattle, pigs, horses, sheep, cats, and dogs. Preferably, the subject is a human.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, a “PCSK9-related diseases or disorder” includes any disease or disorder that can be treated by a PCSK9 RNAi agent (i.e., that a reduction in expression of the PCSK9 gene and thereby a reduction in the amount of PCSK9 protein found in the cell or tissue can provide a therapeutic benefit to the subject), including but not limited hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), or any other disease or disorder in which a reduction in low-density lipoprotein cholesterol (LDL-C) is desired or would benefit the subject or patient.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol

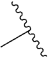

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Other objects, features, aspects, and advantages of the invention will be apparent from the following detailed description, accompanying figures, and from the claims.

### RNAi Agents

Described herein are RNAi agents for inhibiting expression of a PCSK9 gene. Each PCSK9 RNAi agent comprises a sense strand and an antisense strand. The sense strand can be 15 to 49 nucleotides in length. The antisense strand can be 18 to 49 nucleotides in length. The sense and antisense strands can be either the same length or they can be different lengths. In some embodiments, the sense and antisense strands are each independently 21 to 27 nucleotides in length. In some embodiments, both the sense and antisense strands are each 21-26 nucleotides in length. In some embodiments, the sense and antisense strands are each 21-24 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent antisense strands are each independently 18, 19, 20, 21, 22, 23, 24, 25, 26, 27, 28, 29, or 30 nucleotides in length. In some embodiments, the RNAi agent sense strands are each independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The sense and antisense strands are annealed to form a duplex, and in some embodiments, a double-stranded RNAi agent has a duplex length of about 15, 16, 17, 18, 19, 20, 21, 22, 23, 24, 25, 26, 27, 28, 29, or 30 nucleotides.

Examples of nucleotide sequences used in forming PCSK9 RNAi agents are provided in Tables 2, 3, 4, 5C, 7A, 7B, and 8. Examples of RNAi agent duplexes, that include the sense strand and antisense strand sequences in Tables 2, 3, 4, 5C, 7A, and 7B, are shown in Tables 5A, 5B, 5C., and 8.

In some embodiments, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementaiy).

A sense strand of the PCSK9 RNAi agents described herein includes at least 15 consecutive nucleotides that have at least 85% identity to a core stretch sequence (also referred to herein as a “core stretch” or “core sequence”) of the same number of nucleotides in a PCSK9 mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the PCSK9 mRNA target. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length. In some embodiments, this sense strand core stretch is 21 nucleotides in length.

An antisense strand of a PCSK9 RNAi agent described herein includes at least 15 consecutive nucleotides that have at least 85% complementarity to a core stretch of the same number of nucleotides in a PCSK9 mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the PCSK9 mRNA target. In some embodiments, this antisense strand core stretch is 15, 16, 17, 18, 19, 20, 21, 22, or 23 nucleotides in length. In some embodiments, this antisense strand core stretch is 21 nucleotides in length. In some embodiments, this antisense strand core stretch is 19 nucleotides in length. A sense strand core stretch sequence can be the same length as a corresponding antisense core sequence or it can be a different length.

The PCSK9 RNAi agent sense and antisense strands anneal to form a duplex. A sense strand and an antisense strand of a PCSK9 RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, at least 23, at least 24, or at least 25 nucleotides that is at least 85% or 100% complementary to a corresponding 15, 16, 17, 18, 19, 20, 21, 22, 23, 24, or 25 nucleotide sequence of the antisense strand core stretch sequence (i.e., the sense and antisense core stretch sequences of a PCSK9 RNAi agent have a region of at least 15, at least 16, at least 17, at least 18, at least 19, at least 20, at least 21, at least 22, at least 23, at least 24, or at least 25 nucleotides that is at least 85% base paired or 100% base paired.)

In some embodiments, the antisense strand of a PCSK9 RNAi agent disclosed herein differs by 0, 1, 2, or 3 nucleotides from any of the antisense strand sequences in Table 2, Table 3, Table 5C, Table 7A, or Table 8. In some embodiments, the sense strand of a PCSK9 RNAi agent disclosed herein differs by 0, 1, 2, or 3 nucleotides from any of the sense strand sequences in Table 2, Table 4, Table 5C, Table 7B, or Table 8.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the PCSK9 mRNA. The sense strand additional nucleotides, if present, may or may not be identical to the corresponding sequence in the PCSK9 mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a PCSK9 RNAi agent has an antisense strand having a 3′ extension and a sense strand having a 5′ extension. In some embodiments, the extension nucleotide(s) are unpaired and form an overhang. As used herein and in the art, an “overhang” refers to an extension of a stretch of one or more unpaired nucleotides located at a terminal end of either the sense strand or the antisense strand that does not form part of the hybridized or duplexed portion of an RNAi agent disclosed herein.

In some embodiments, a PCSK9 RNAi agent comprises an antisense strand having a 3′ extension of 1, 2, 3, 4, 5, or 6 nucleotides in length. In other embodiments, a PCSK9 RNAi agent comprises an antisense strand having a 3′ extension of 1, 2, or 3 nucleotides in length. In some embodiments, one or more of the antisense strand extension nucleotides comprise nucleotides that are complementary to the corresponding PCSK9 mRNA sequence. In some embodiments, one or more of the antisense strand extension nucleotides comprise nucleotides that are not complementary to the corresponding PCSK9 mRNA sequence.

In some embodiments, a PCSK9 RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the PCSK9 mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a PCSK9 RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the PCSK9 mRNA sequence.

Examples of sequences used in forming PCSK9 RNAi agents are provided in Tables 2, 3, 4, 5C, 7A, 7B, and 8. In some embodiments, a PCSK9 RNAi agent antisense strand includes a sequence of any of the sequences in Tables 2, 3, 5C, 7A, or 8. In certain embodiments, a PCSK9 RNAi agent antisense strand comprises or consists of any one of the modified sequences in Table 3. In some embodiments, a PCSK9 RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2, 3, 5C, 7A, or 8. In some embodiments, a PCSK9 RNAi agent sense strand includes the sequence of any of the sequences in Tables 2, 4, 5C, 7B, or 8. In some embodiments, a PCSK9 RNAi agent sense strand includes the sequence of nucleotides (from 5′ end - 3′ end) 1-18, 1-19, 1-20, 1-21, 2-19, 2-20, 2-21.,3-20, 3-21, or 4-21 of any of the sequences in Tables 2, 4, 5C, 7B, or 8. In certain embodiments, a PCSK9 RNAi agent sense strand comprises or consists of a modified sequence of any one of the modified sequences in Table 4.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The PCSK9 RNAi agents disclosed herein may also be comprised of one or more modified nucleotides. In some embodiments, substantially all of the nucleotides of the sense strand and substantially all of the nucleotides of the antisense strand of the PCSK9 RNAi agent are modified nucleotides. The PCSK9 RNAi agents disclosed herein may further be comprised of one or more modified intemucleoside linkages, e.g., one or more phosphorothioate linkages. In some embodiments, a PCSK9 RNAi agent contains one or more modified nucleotides and one or more modified intemucleoside linkages. In some embodiments, a 2′-modified nucleotide is combined with modified intemucleoside linkage.

In some embodiments, a PCSK9 RNAi agent is prepared or provided as a salt, mixed salt, or a free-acid. In some embodiments, a PCSK9 RNAi agent is prepared as a pharmaceutically acceptable salt. In some embodiments, a PCSK9 RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon αctivity in humans upon administering of the oligonucleotide construct.

In some embodiments, a PCSK9 RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues or unlocked nucleotides), locked nucleotides, 3′-O-methoxy (2′ intemucleoside linked) nucleotides, 2′-F-Arabino nucleotides, 5′-Me, 2′-fluoro nucleotide, morpholino nucleotides, vinylphosphonate deoxyribonucleotides, vinylphosphonate-containing nucleotides, and cyclopropylphosphonate-containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to herein or in the art as 2′-methoxy nucleotides), 2′-fluoro nucleotides (also referred to herein or in the art as 2′-deoxy-2′-fluoro nucleotides), 2′-deoxy nucleotides, 2′-methoxyethyl(2′-O-2-methoxylethyl) nucleotides (also referred herein or in the art as 2′-MOE nucleotides), 2′-amino nucleotides, and 2′-alkyl nucleotides. It is not necessary for all positions in a given compound to be uniformly modified. Conversely, more than one modification can be incorporated in a single PCSK9 RNAi agent or even in a single nucleotide thereof. The PCSK9 RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrinidines and N-2, N-6 and 0-6 substituted purines, (e.g., 2-aminopropyladenine, 5-propynyluracil, or 5-propynylcytosine), 5-nethylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulflydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 6 herein.

### Modified Internucleoside Linkages

In some embodiments, one or more nucleotides of a PCSK9 RNAi agent are linked by non-standard linkages or backbones (i.e., modified intemucleoside linkages or modified backbones). Modified intemucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified intemucleoside linkage or backbone lacks a phosphorus atom. Modified intemucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified intemucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a PCSK9 RNAi agent can contain 1, 2, 3, 4, 5, or 6 phosphorothioate linkages, an antisense strand of a PCSK9 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a PCSK9 RNAi agent can contain 1, 2, 3, or 4 phosphorothioate linkages, an antisense strand of a PCSK9 RNAi agent can contain 1, 2, 3, or 4 phosphorothioate linkages, or both the sense strand and the antisense strand independently can contain 1, 2, 3, or 4 phosphorothioate linkages.

In some embodiments, a PCSK9 RNAi agent sense strand contains at least two phosphorothioate intemucleoside linkages. In some embodiments, the phosphorothioate intemucleoside linkages are between the nucleotides at positions 1-3 from the 3′ end of the sense strand. In some embodiments, one phosphorothioate intemucleoside linkage is at the 5′ end of the sense strand nucleotide sequence, and another phosphorothioate linkage is at the 3′ end of the sense strand nucleotide sequence. In some embodiments, two phosphorothioate intemucleoside linkages are located at the 5′ end of the sense strand, and another phosphorothioate linkage is at the 3′ end of the sense strand. In some embodiments, the sense strand does not include any phosphorothioate intemucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a PCSK9 RNAi agent antisense strand contains four phosphorothioate intemucleoside linkages. In some embodiments, the four phosphorothioate intemucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate intemucleoside linkages are located between positions 1-4 from the 5′ end of the antisense strand, and a fourth phosphorothioate intemucleoside linkage is located between positions 20-21 from the 5′ end of the antisense strand. In some embodiments, a PCSK9 RNAi agent contains at least three or four phosphorothioate intemucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues. (See, e.g., F. Czauderna, Nucleic Acids Res., 2003, 31(11), 2705-16; U.S. Pat. No. 5,998,203). Capping residues are generally known in the art, and include, for example, inverted abasic residues as well as carbon chains such as a terminal PCSK9H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 6 below.

### PCSK9 RNAi Agents

The PCSK9 RNAi agents disclosed herein are designed to target specific positions on a PCSK9 gene (e.g., SEQ ID NO:1).

| NM_174936.4, Homo sapiens, proprotein convertase subtilisin/ | |
|---|---|
| kexin type 9 (PCSK9), mRNA transcript | |
| (SEQ ID NO: 1): | |
| 1 | agcgacgtcg aggcgctcat ggttgcaggc gggcgccgcc gttcagttca gggtctgagc |
| 61 | ctggaggagt gagccaggca gtgagactgg ctcgggcggg ccgggacgcg tcgttgcagc |
| 121 | agcggctccc agctcccagc caggattccg cgcgcccctt cacgcgccct gctcctgaac |
| 181 | ttcagctcct gcacagtcct ccccaccgca aggctcaagg cgccgccggc gtggaccgcg |
| 241 | cacggcctct aggtctcctc gccaggacag caacctctcc cctggccctc atgggcaccg |
| 301 | tcagctccag gcggtcctgg tggccgctgc cactgctgct gctgctgctg ctgctcctgg |
| 361 | gtcccgcggg cgcccgtgcg caggaggacg aggacggcga ctacgaggag ctggtgctag |
| 421 | ccttgcgttc cgaggaggac ggcctggccg aagcacccga gcacggaacc acagccacct |
| 481 | tccaccgctg cgccaaggat ccgtggaggt tgcctggcac ctacgtggtg gtgctgaagg |
| 541 | aggagaccca cctctcgcag tcagagcgca ctgcccgccg cctgcaggcc caggctgccc |
| 601 | gccggggata cctcaccaag atcctgcatg tcttccatgg ccttcttcct ggcttcctgg |
| 661 | tgaagatgag tggcgacctg ctggagctgg ccttgaagtt gccccatgtc gactacatcg |
| 721 | aggaggactc ctctgtcttt gcccagagca tcccgtggaa cctggagcgg attacccctc |
| 781 | cacggtaccg ggcggatgaa taccagcccc ccgacggagg cagcctggtg gaggtgtatc |
| 841 | tcctagacac cagcatacag agtgaccacc gggaaatcga gggcagggtc atggtcaccg |
| 901 | acttcgagaa tgtgcccgag gaggacggga cccgcttcca cagacaggcc agcaagtgtg |
| 961 | acagtcatgg cacccacctg gcaggggtgg tcagcggccg ggatgccggc gtggccaagg |
| 1021 | gtgccagcat gcgcagcctg cgcgtgctca actgccaagg gaagggcacg gttagcggca |
| 1081 | ccctcatagg cctggagttt attcggaaaa gccagctggt ccagcctgtg gggccactgg |
| 1141 | tggtgctgct gcccctggcg ggtgggtaca gccgcgtcct caacgccgcc tgccagcgcc |
| 1201 | tggcgagggc tggggtcgtg ctggtcaccg ctgccggcaa cttccgggac gatgcctgcc |
| 1261 | tctactcccc agcctcagct cccgaggtca tcacagttgg ggccaccaat gcccaagacc |
| 1321 | agccggtgac cctggggact ttggggacca actttggccg ctgtgtggac ctctttgccc |
| 1381 | caggggagga catcattggt gcctccagcg actgcagcac ctgctttgtg tcacagagtg |
| 1441 | ggacatcaca ggctgctgcc cacgtggctg gcattgcagc catgatgctg tctgccgagc |
| 1501 | cggagctcac cctggccgag ttgaggcaga gactgatcca cttctctgcc aaagatgtca |
| 1561 | tcaatgaggc ctggttccct gaggaccagc gggtactgac ccccaacctg gtggccgccc |
| 1621 | tgccccccag cacccatggg gcaggttggc agctgttttg caggactgta tggtcagcac |
| 1681 | actcggggcc tacacggatg gccacagccg tcgcccgctg cgccccagat gaggagctgc |
| 1741 | tgagctgctc cagtttctcc aggagtggga agcggcgggg cgagcgcatg gaggcccaag |
| 1801 | ggggcaagct ggtctgccgg gcccacaacg cttttggggg tgagggtgtc tacgccattg |
| 1861 | ccaggtgctg cctgctaccc caggccaact gcagcgtcca cacagctcca ccagctgagg |
| 1921 | ccagcatggg gacccgtgtc cactgccacc aacagggcca cgtcctcaca ggctgcagct |
| 1981 | cccactggga ggtggaggac cttggcaccc acaagccgcc tgtgctgagg ccacgaggtc |
| 2041 | agcccaacca gtgcgtgggc cacagggagg ccagcatcca cgcttcctgc tgccatgccc |
| 2101 | caggtctgga atgcaaagtc aaggagcatg gaatcccggc ccctcaggag caggtgaccg |
| 2161 | tggcctgcga ggagggctgg accctgactg gctgcagtgc cctccctggg acctcccacg |
| 2221 | tcctgggggc ctacgccgta gacaacacgt gtgtagtcag gagccgggac gtcagcacta |
| 2281 | caggcagcac cagcgaaggg gccgtgacag ccgttgccat ctgctgccgg agccggcacc |
| 2341 | tggcgcaggc ctcccaggag ctccagtgac agccccatcc caggatgggt gtctggggag |
| 2401 | ggtcaagggc tggggctgag ctttaaaatg gttccgactt gtccctctct cagccctcca |
| 2461 | tggcctggca cgaggggatg gggatgcttc cgcctttccg gggctgctgg cctggccctt |
| 2521 | gagtggggca gcctccttgc ctggaactca ctcactctgg gtgcctcctc cccaggtgga |
| 2581 | ggtgccagga agctccctcc ctcactgtgg ggcatttcac cattcaaaca ggtcgagctg |
| 2641 | tgctcgggtg ctgccagctg ctcccaatgt gccgatgtcc gtgggcagaa tgacttttat |
| 2701 | tgagctcttg ttccgtgcca ggcattcaat cctcaggtct ccaccaagga ggcaggattc |
| 2761 | ttcccatgga taggggaggg ggcggtaggg gctgcaggga caaacatcgt tggggggtga |
| 2821 | gtgtgaaagg tgctgatggc cctcatctcc agctaactgt ggagaagccc ctgggggctc |
| 2881 | cctgattaat ggaggcttag ctttctggat ggcatctagc cagaggctgg agacaggtgc |
| 2941 | gcccctggtg gtcacaggct gtgccttggt ttcctgagcc acctttactc tgctctatgc |
| 3001 | caggctgtgc tagcaacacc caaaggtggc ctgcggggag ccatcaccta ggactgactc |
| 3061 | ggcagtgtgc agtggtgcat gcactgtctc agccaacccg ctccactacc cggcagggta |
| 3121 | cacattcgca cccctacttc acagaggaag aaacctggaa ccagaggggg cgtgcctgcc |
| 3181 | aagctcacac agcaggaact gagccagaaa cgcagattgg gctggctctg aagccaagcc |
| 3241 | tcttcttact tcacccggct gggctcctca tttttacggg taacagtgag gctgggaagg |
| 3301 | ggaacacaga ccaggaagct cggtgagtga tggcagaacg atgcctgcag gcatggaact |
| 3361 | ttttccgtta tcacccaggc ctgattcact ggcctggcgg agatgcttct aaggcatggt |
| 3421 | cgggggagag ggccaacaac tgtccctcct tgagcaccag ccccacccaa gcaagcagac |
| 3481 | atttatcttt tgggtctgtc ctctctgttg cctttttaca gccaactttt ctagacctgt |
| 3541 | tttgcttttg taacttgaag atatttattc tgggttttgt agcattttta ttaatatggt |
| 3601 | gactttttaa aataaaaaca aacaaacgtt gtcctaa |

As defined herein, an antisense strand sequence is designed to target a PCSK9 gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target a PCSK9 gene at position 3536 requires that when base pairing to the gene, the 5′ terminal nucleobase of the antisense strand is aligned with position 3556 of the PCSK9 gene.

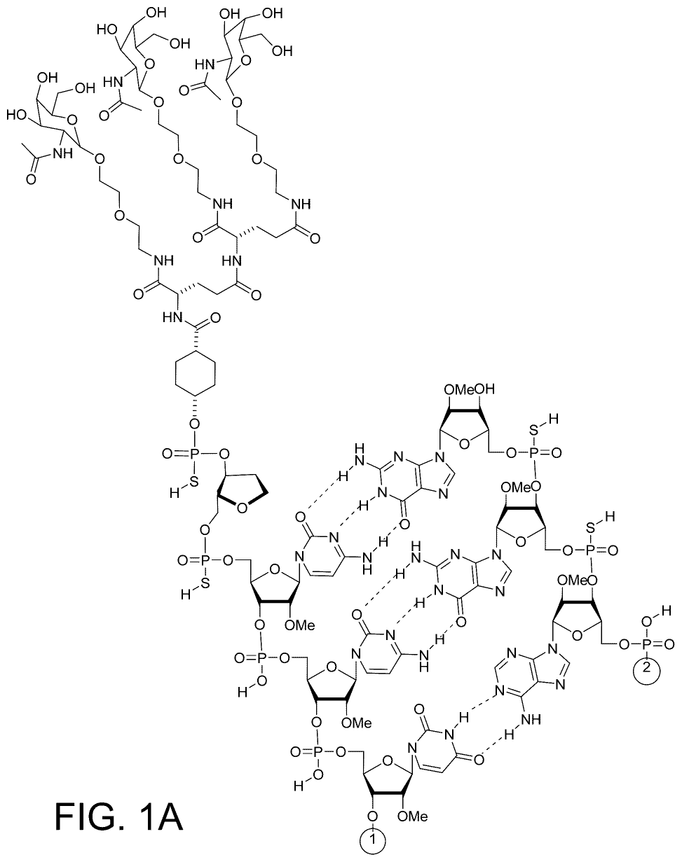

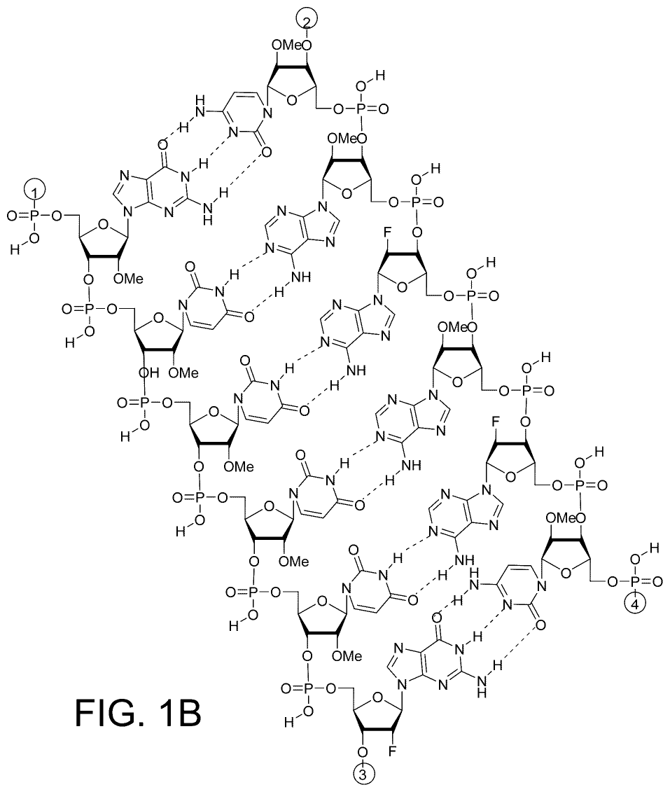

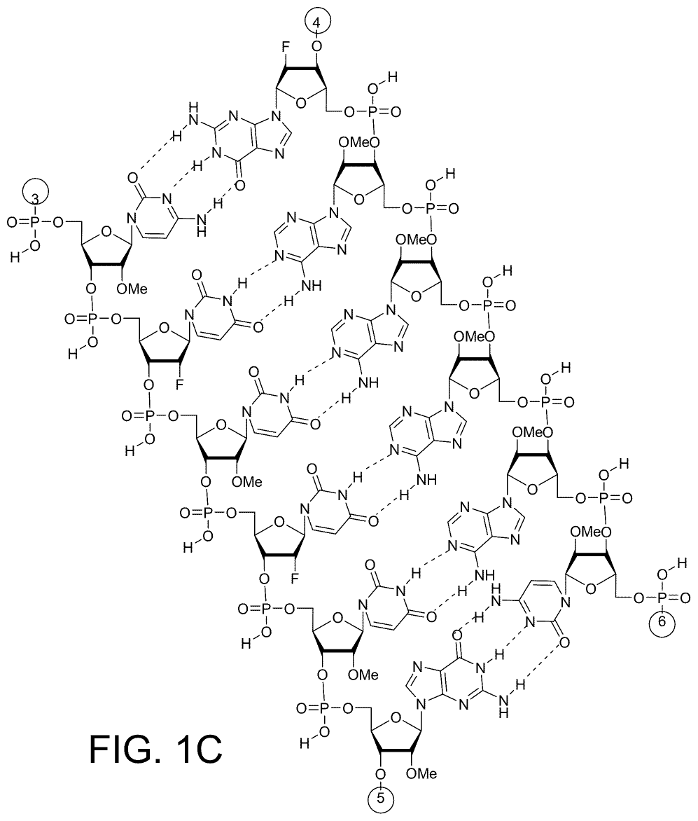

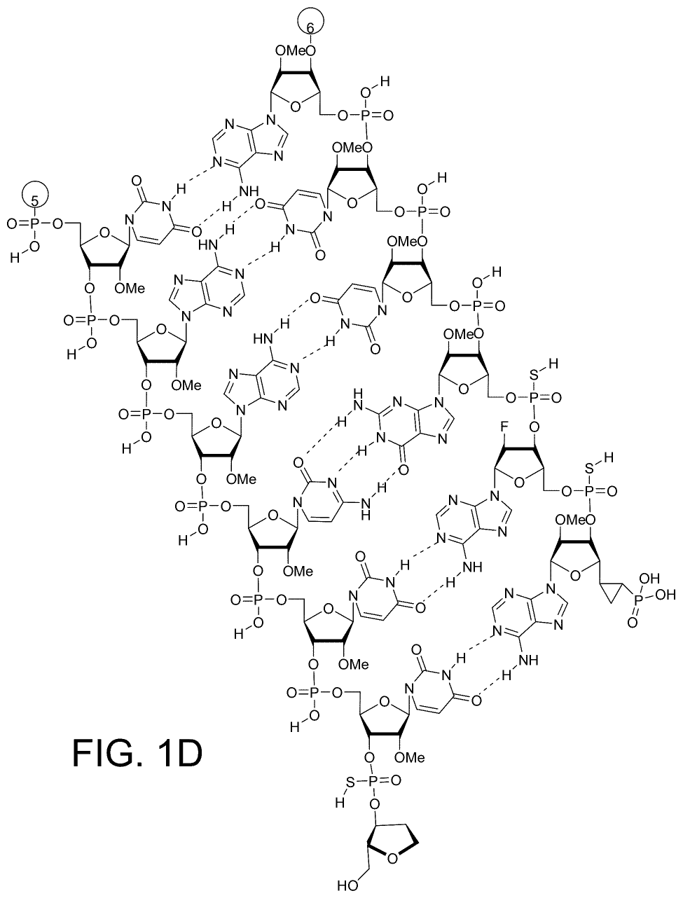

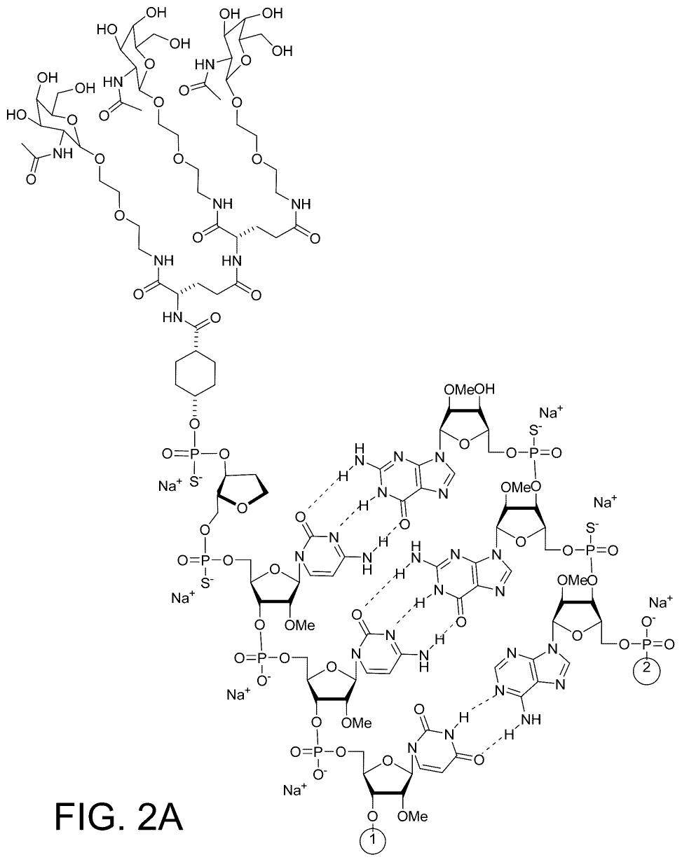

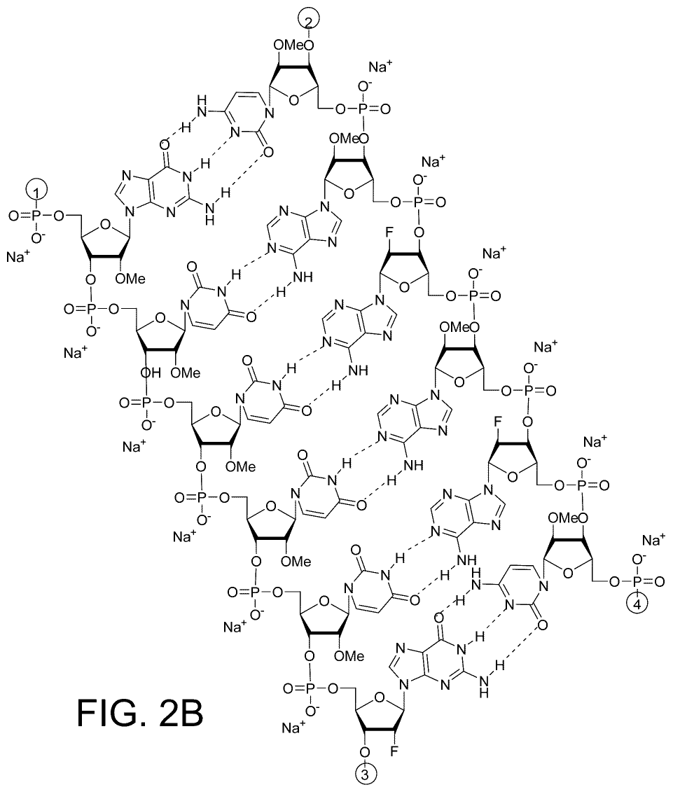

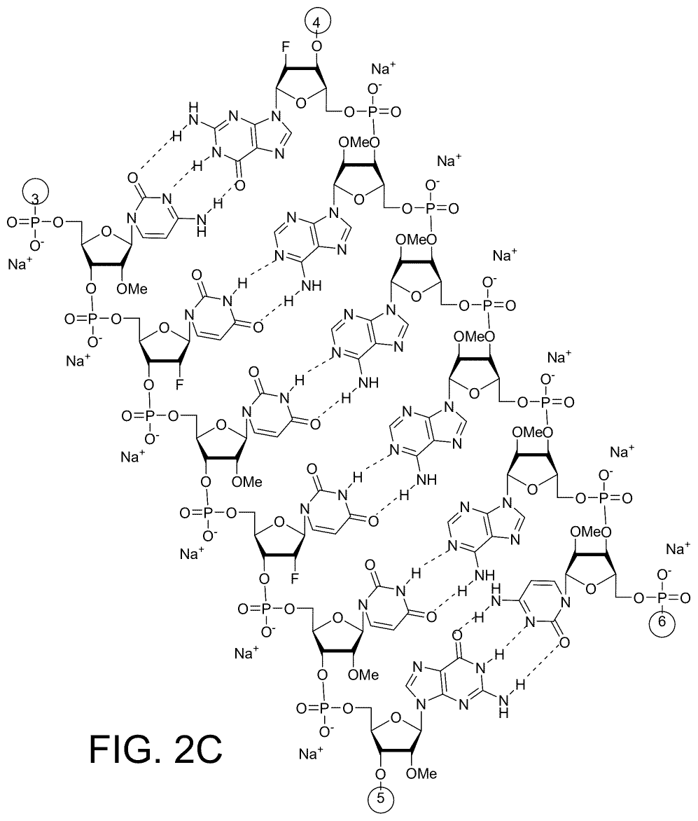

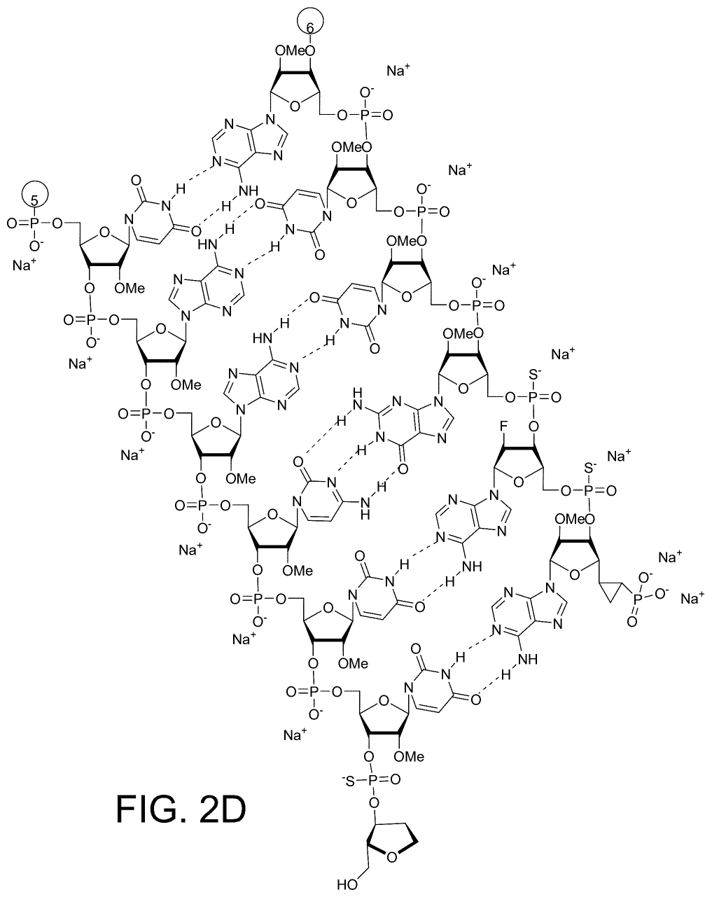

As provided herein, a PCSK9 RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 15 consecutive nucleotides. For example, for a PCSK9 RNAi agent disclosed herein that is designed to target position 3536 of a PCSK9 gene, the 5′ terminal nucleobase of the antisense strand of the of the PCSK9 RNAi agent must be aligned with position 3556 of the gene; however, the 5′ terminal nucleobase of the antisense strand may be, but is not required to be, complementary to position 3556 of a PCSK9 gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 15 consecutive nucleotides. As shown by, among other things, the examples disclosed herein and as is well known in the art, the specific site of binding of the gene by the antisense strand of the PCSK9 RNAi agent (e.g., whether the PCSK9 RNAi agent is designed to target a PCSK9 gene at position 3536 or at some other position) is important to the level of inhibition achieved by the PCSK9 RNAi agent as well as the toxicity profile achieved by the molecule. (See, e.g., Kamola et al., The siRNANon-seedRegion andIts Target Sequences are Auxiliary Determinants of Off-Target Effects, PLOS Computational Biology, 11(12), FIG. 1 (2015)).

In some embodiments, the PCSK9 RNAi agents disclosed herein target a PCSK9 gene at or near the positions of the PCSK9 gene sequence shown in Table 1. In some embodiments, the antisense strand of a PCSK9 RNAi agent disclosed herein includes a core stretch sequence that is fully, substantially, or at least partially complementary to a target PCSK9 21-mer sequence disclosed in Table 1.

TABLE 1
PCSK9 21-mer mRNA Target Sequences (taken
from homo sapiens PCSK9, mRNA, GenBank
NM_174936.4 (SEQ ID NO: 1))

| | | Corre- | |
|---|---|---|---|
| | | sponding | Targeted |
| | | Positions | Gene |
| | | of | Position |
| | | Sequence | (as |
| SEQ | PCSK9 21-mer | on | referred |
| ID | Target Sequences | SEQ ID | to |
| No. | (5′ → 3′) | NO: 1 | herein) |
| 2 | GGGGAUACCUCACCAAGAUCC | 604-624 | 604 |
| 3 | GGGAUACCUCACCAAGAUCCU | 605-625 | 605 |
| 4 | CUCACCAAGAUCCUGCAUGUC | 612-632 | 612 |
| 5 | CACCAAGAUCCUGCAUGUCUU | 614-634 | 614 |
| 6 | GGUCAUGGUCACCGACUUCGA | 887-907 | 887 |
| 7 | GCCUGGAGUUUAUUCGGAAAA | 1090-1110 | 1090 |
| 8 | UCAUUGGUGCCUCCAGCGACU | 1393-1413 | 1393 |
| 9 | UCUCUGCCAAAGAUGUCAUCA | 1543-1563 | 1543 |
| 10 | UGCCAGGUGCUGCCUGCUACC | 1859-1879 | 1859 |
| 11 | GGAACACAGACCAGGAAGCUC | 3301-3321 | 3301 |
| 12 | ACCCAAGCAAGCAGACAUUUA | 3465-3485 | 3465 |
| 13 | CCCAAGCAAGCAGACAUUUAU | 3466-3486 | 3466 |
| 14 | CAAGCAAGCAGACAUUUAUCU | 3468-3488 | 3468 |
| 15 | AGCAGACAUUUAUCUUUUGGG | 3474-3494 | 3474 |
| 16 | UUUAUCUUUUGGGUCUGUCCU | 3482-3502 | 3482 |
| 17 | UCUUUUGGGUCUGUCCUCUCU | 3486-3506 | 3486 |
| 18 | CCUCUCUGUUGCCUUUUUACA | 3500-3520 | 3500 |
| 19 | CUCUCUGUUGCCUUUUUACAG | 3501-3521 | 3501 |
| 20 | UCUCUGUUGCCUUUUUACAGC | 3502-3522 | 3502 |
| 21 | CAACUUUUCUAGACCUGUUUU | 3523-3543 | 3523 |
| 22 | CUAGACCUGUUUUGCUUUUGU | 3531-3551 | 3531 |
| 23 | UAGACCUGUUUUGCUUUUGUA | 3532-3552 | 3532 |
| 24 | GACCUGUUUUGCUUUUGUAAC | 3534-3554 | 3534 |
| 25 | CCUGUUUUGCUUUUGUAACUU | 3536-3556 | 3536 |
| 26 | UUUUGCUUUUGUAACUUGAAG | 3540-3560 | 3540 |
| 27 | UUUUGUAACUUGAAGAUAUUU | 3546-3566 | 3546 |
| 28 | UUUGUAACUUGAAGAUAUUUA | 3547-3567 | 3547 |
| 29 | UUGUAACUUGAAGAUAUUUAU | 3548-3568 | 3548 |

In some embodiments, a PCSK9 RNAi agent includes an antisense strand wherein position 21 of the antisense strand (5′→3′) is capable of forming a base pair with position 1 of a 21-mer target sequence disclosed in Table 1. In some embodiments, a PCSK9 RNAi agent includes an antisense strand wherein position 1 of the antisense strand (5′→3′) is capable of forming a base pair with position 21 of the 21-mer target sequence disclosed in Table 1.

In some embodiments, a PCSK9 RNAi agent includes an antisense strand wherein position 2 of the antisense strand (5′→3′) is capable of forming a base pair with position 20 of the 21-mer target sequence disclosed in Table 1. In some embodiments, a PCSK9 RNAi agent includes an antisense strand wherein positions 2 through 18 of the antisense strand (5′ 4 3′) are capable of forming base pairs with each of the respective complementary bases located at positions 18 through 2 of the 21-mer target sequence disclosed in Table 1.

For the RNAi agents disclosed herein, the nucleotide at position 1 of the antisense strand (from 5′ end→3′ end) can be perfectly complementary to the PCSK9 gene, or can be non-complementary to the PCSK9 gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a PCSK9 RNAi agent antisense strand comprises the sequence of nucleotides (from 5′ end→3′ end) 2-18, 2-19, 2-20, or 2-21 of any of the antisense strand sequences in Table 2, Table 3, Table 5C, Table 7A, or Table 8. In some embodiments, a PCSK9 RNAi sense strand comprises the sequence of nucleotides (from 5′ end - 3′ end) 3-21, 2-21, 1-21, 3-20, 2-20, 1-20, 3-19, 2-19, 1-19, 3-18, 2-18, or 1-18 of any of the sense strand sequences in Table 2, Table 4, Table 5C, Table 7B, or Table 8.

In some embodiments, a PCSK9 RNAi agent antisense strand comprises the sequence of nucleotides (from 5′ end - 3′ end) 2-18, 2-19, 2-20, or 2-21 of any of the antisense strand sequences of Table 2, Table 3, Table 5C, Table 7A, or Table 8. In some embodiments, a PCSK9 RNAi sense strand comprises the sequence of nucleotides (from 5′ end→3′ end) 3-21, 2-21, 1-21, 3-20, 2-20, 1-20, 3-19, 2-19, 1-19, 3-18, 2-18, or 1-18 of any of the sense strand sequences of Table 2, Table 4, Table 5C, Table 7B, or Table 8.

In some embodiments, a PCSK9 RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 3-21, 2-21, 1-21, 3-20, 2-20, 1-20, 3-19, 2-19, 1-19, 3-18, 2-18, or 1-18 of any of the sense strand sequences in Table 2 or Table 4.

In some embodiments, a PCSK9 RNAi agent is comprised of (i) an antisense strand comprising the sequence of nucleotides (from 5′ end→3′ end) 2-18 or 2-19 of any of the antisense strand sequences of Table 2 or Table 3, and (ii) a sense strand comprising the sequence of nucleotides (from 5′ end→3′ end) 3-21, 2-21, 1-21, 3-20, 2-20, 1-20, 3-19, 2-19, 1-19, 3-18, 2-18, or 1-18 of any of the sense strand sequences of Table 2 or Table 4.

In some embodiments, the PCSK9 RNAi agents include core 21-mer nucleotide sequences shown in the following Table 2.

TABLE 2
PCSK9 RNAi Agent Antisense Strand and Sense Strand
Core Stretch Base Sequences (N = any nucleobase;)

| | Antisense Strand | | Sense Strand | Corresponding | |
|---|---|---|---|---|---|
| | Base Sequence | | Base Sequence | Positions of | |
| SEQ | (5′ → 3′) (Shown | SEQ | (5′ → 3′) (Shown | Identified | Targeted |
| ID | as an Unmodified | ID | as an Unmodified | Sequence on | Gene |
| No. | Nucleotide Sequence) | No. | Nucleotide Sequence) | SEQ ID NO: 1 | Position |
| 30 | GGAUCUUGGUGAGGUAUCCCC | 158 | GGGGAUACCUCACCAAGAUCC | 604-624 | 604 |
| 31 | UGAUCUUGGUGAGGUAUCCCC | 159 | GGGGAUACCUCACCAAGAUCA | 604-624 | 604 |
| 32 | AGAUCUUGGUGAGGUAUCCCC | 160 | GGGGAUACCUCACCAAGAUCU | 604-624 | 604 |
| 33 | NGAUCUUGGUGAGGUAUCCCC | 161 | GGGGAUACCUCACCAAGAUCN | 604-624 | 604 |
| 34 | NGAUCUUGGUGAGGUAUCCCN | 162 | NGGGAUACCUCACCAAGAUCN | 604-624 | 604 |
| 35 | AGGAUCUUGGUGAGGUAUCCC | 163 | GGGAUACCUCACCAAGAUCCU | 605-625 | 605 |
| 36 | UGGAUCUUGGUGAGGUAUCCC | 164 | GGGAUACCUCACCAAGAUCCA | 605-625 | 605 |
| 37 | NGGAUCUUGGUGAGGUAUCCC | 165 | GGGAUACCUCACCAAGAUCCN | 605-625 | 605 |
| 38 | NGGAUCUUGGUGAGGUAUCCN | 166 | NGGAUACCUCACCAAGAUCCN | 605-625 | 605 |
| 39 | GACAUGCAGGAUCUUGGUGAG | 167 | CUCACCAAGAUCCUGCAUGUC | 612-632 | 612 |
| 40 | UACAUGCAGGAUCUUGGUGAG | 168 | CUCACCAAGAUCCUGCAUGUA | 612-632 | 612 |
| 41 | AACAUGCAGGAUCUUGGUGAG | 169 | CUCACCAAGAUCCUGCAUGUU | 612-632 | 612 |
| 42 | NACAUGCAGGAUCUUGGUGAG | 170 | CUCACCAAGAUCCUGCAUGUN | 612-632 | 612 |
| 43 | NACAUGCAGGAUCUUGGUGAN | 171 | NUCACCAAGAUCCUGCAUGUN | 612-632 | 612 |
| 44 | AAGACAUGCAGGAUCUUGGUG | 172 | CACCAAGAUCCUGCAUGUCUU | 614-634 | 614 |
| 45 | UAGACAUGCAGGAUCUUGGUG | 173 | CACCAAGAUCCUGCAUGUCUA | 614-634 | 614 |
| 46 | NAGACAUGCAGGAUCUUGGUG | 174 | CACCAAGAUCCUGCAUGUCUN | 614-634 | 614 |
| 47 | NAGACAUGCAGGAUCUUGGUN | 175 | NACCAAGAUCCUGCAUGUCUN | 614-634 | 614 |
| 48 | UCGAAGUCGGUGACCAUGACC | 176 | GGUCAUGGUCACCGACUUCGA | 887-907 | 887 |
| 49 | ACGAAGUCGGUGACCAUGACC | 177 | GGUCAUGGUCACCGACUUCGU | 887-907 | 887 |
| 50 | NCGAAGUCGGUGACCAUGACC | 178 | GGUCAUGGUCACCGACUUCGN | 887-907 | 887 |
| 51 | NCGAAGUCGGUGACCAUGACN | 179 | NGUCAUGGUCACCGACUUCGN | 887-907 | 887 |
| 52 | UUUUCCGAAUAAACUCCAGGC | 180 | GCCUGGAGUUUAUUCGGAAAA | 1090-1110 | 1090 |
| 53 | AUUUCCGAAUAAACUCCAGGC | 181 | GCCUGGAGUUUAUUCGGAAAU | 1090-1110 | 1090 |
| 54 | NUUUCCGAAUAAACUCCAGGC | 182 | GCCUGGAGUUUAUUCGGAAAN | 1090-1110 | 1090 |
| 55 | NUUUCCGAAUAAACUCCAGGN | 183 | NCCUGGAGUUUAUUCGGAAAN | 1090-1110 | 1090 |
| 56 | AGUCGCUGGAGGCACCAAUGA | 184 | UCAUUGGUGCCUCCAGCGACU | 1393-1413 | 1393 |
| 57 | UGUCGCUGGAGGCACCAAUGA | 185 | UCAUUGGUGCCUCCAGCGACA | 1393-1413 | 1393 |
| 58 | NGUCGCUGGAGGCACCAAUGA | 186 | UCAUUGGUGCCUCCAGCGACN | 1393-1413 | 1393 |
| 59 | NGUCGCUGGAGGCACCAAUGN | 187 | NCAUUGGUGCCUCCAGCGACN | 1393-1413 | 1393 |
| 60 | UGAUGACAUCUUUGGCAGAGA | 188 | UCUCUGCCAAAGAUGUCAUCA | 1543-1563 | 1543 |
| 61 | AGAUGACAUCUUUGGCAGAGA | 189 | UCUCUGCCAAAGAUGUCAUCU | 1543-1563 | 1543 |
| 62 | NGAUGACAUCUUUGGCAGAGA | 190 | UCUCUGCCAAAGAUGUCAUCN | 1543-1563 | 1543 |
| 63 | NGAUGACAUCUUUGGCAGAGN | 191 | NCUCUGCCAAAGAUGUCAUCN | 1543-1563 | 1543 |
| 64 | GGUAGCAGGCAGCACCUGGCA | 192 | UGCCAGGUGCUGCCUGCUACC | 1859-1879 | 1859 |
| 65 | UGUAGCAGGCAGCACCUGGCA | 193 | UGCCAGGUGCUGCCUGCUACA | 1859-1879 | 1859 |
| 66 | AGUAGCAGGCAGCACCUGGCA | 194 | UGCCAGGUGCUGCCUGCUACU | 1859-1879 | 1859 |
| 67 | NGUAGCAGGCAGCACCUGGCA | 195 | UGCCAGGUGCUGCCUGCUACN | 1859-1879 | 1859 |
| 68 | NGUAGCAGGCAGCACCUGGCN | 196 | NGCCAGGUGCUGCCUGCUACN | 1859-1879 | 1859 |
| 69 | GAGCUUCCUGGUCUGUGUUCC | 197 | GGAACACAGACCAGGAAGCUC | 3301-3321 | 3301 |
| 70 | UAGCUUCCUGGUCUGUGUUCC | 198 | GGAACACAGACCAGGAAGCUA | 3301-3321 | 3301 |
| 71 | AAGCUUCCUGGUCUGUGUUCC | 199 | GGAACACAGACCAGGAAGCUU | 3301-3321 | 3301 |
| 72 | NAGCUUCCUGGUCUGUGUUCC | 200 | GGAACACAGACCAGGAAGCUN | 3301-3321 | 3301 |
| 73 | NAGCUUCCUGGUCUGUGUUCN | 201 | NGAACACAGACCAGGAAGCUN | 3301-3321 | 3301 |
| 74 | UAAAUGUCUGCUUGCUUGGGU | 202 | ACCCAAGCAAGCAGACAUUUA | 3465-3485 | 3465 |
| 75 | AAAAUGUCUGCUUGCUUGGGU | 203 | ACCCAAGCAAGCAGACAUUUU | 3465-3485 | 3465 |
| 76 | NAAAUGUCUGCUUGCUUGGGU | 204 | ACCCAAGCAAGCAGACAUUUN | 3465-3485 | 3465 |
| 77 | NAAAUGUCUGCUUGCUUGGGN | 205 | NCCCAAGCAAGCAGACAUUUN | 3465-3485 | 3465 |
| 78 | AUAAAUGUCUGCUUGCUUGGG | 206 | CCCAAGCAAGCAGACAUUUAU | 3466-3486 | 3466 |
| 79 | UUAAAUGUCUGCUUGCUUGGG | 207 | CCCAAGCAAGCAGACAUUUAA | 3466-3486 | 3466 |
| 80 | NUAAAUGUCUGCUUGCUUGGG | 208 | CCCAAGCAAGCAGACAUUUAN | 3466-3486 | 3466 |
| 81 | NUAAAUGUCUGCUUGCUUGGN | 209 | NCCAAGCAAGCAGACAUUUAN | 3466-3486 | 3466 |
| 82 | AGAUAAAUGUCUGCUUGCUUG | 210 | CAAGCAAGCAGACAUUUAUCU | 3468-3488 | 3468 |
| 83 | UGAUAAAUGUCUGCUUGCUUG | 211 | CAAGCAAGCAGACAUUUAUCA | 3468-3488 | 3468 |
| 84 | NGAUAAAUGUCUGCUUGCUUG | 212 | CAAGCAAGCAGACAUUUAUCN | 3468-3488 | 3468 |
| 85 | NGAUAAAUGUCUGCUUGCUUN | 213 | NAAGCAAGCAGACAUUUAUCN | 3468-3488 | 3468 |
| 86 | CCCAAAAGAUAAAUGUCUGCU | 214 | AGCAGACAUUUAUCUUUUGGG | 3474-3494 | 3474 |
| 87 | UCCAAAAGAUAAAUGUCUGCU | 215 | AGCAGACAUUUAUCUUUUGGA | 3474-3494 | 3474 |
| 88 | ACCAAAAGAUAAAUGUCUGCU | 216 | AGCAGACAUUUAUCUUUUGGU | 3474-3494 | 3474 |
| 89 | NCCAAAAGAUAAAUGUCUGCU | 217 | AGCAGACAUUUAUCUUUUGGN | 3474-3494 | 3474 |
| 90 | NCCAAAAGAUAAAUGUCUGCN | 218 | NGCAGACAUUUAUCUUUUGGN | 3474-3494 | 3474 |
| 91 | AGGACAGACCCAAAAGAUAAA | 219 | UUUAUCUUUUGGGUCUGUCCU | 3482-3502 | 3482 |
| 92 | UGGACAGACCCAAAAGAUAAA | 220 | UUUAUCUUUUGGGUCUGUCCA | 3482-3502 | 3482 |
| 93 | NGGACAGACCCAAAAGAUAAA | 221 | UUUAUCUUUUGGGUCUGUCCN | 3482-3502 | 3482 |
| 94 | NGGACAGACCCAAAAGAUAAN | 222 | NUUAUCUUUUGGGUCUGUCCN | 3482-3502 | 3482 |
| 95 | AGAGAGGACAGACCCAAAAGA | 223 | UCUUUUGGGUCUGUCCUCUCU | 3486-3506 | 3486 |
| 96 | UGAGAGGACAGACCCAAAAGA | 224 | UCUUUUGGGUCUGUCCUCUCA | 3486-3506 | 3486 |
| 97 | NGAGAGGACAGACCCAAAAGA | 225 | UCUUUUGGGUCUGUCCUCUCN | 3486-3506 | 3486 |
| 98 | NGAGAGGACAGACCCAAAAGN | 226 | NCUUUUGGGUCUGUCCUCUCN | 3486-3506 | 3486 |
| 99 | UGUAAAAAGGCAACAGAGAGG | 227 | CCUCUCUGUUGCCUUUUUACA | 3500-3520 | 3500 |
| 100 | AGUAAAAAGGCAACAGAGAGG | 228 | CCUCUCUGUUGCCUUUUUACU | 3500-3520 | 3500 |
| 101 | NGUAAAAAGGCAACAGAGAGG | 229 | CCUCUCUGUUGCCUUUUUACN | 3500-3520 | 3500 |
| 102 | NGUAAAAAGGCAACAGAGAGN | 230 | NCUCUCUGUUGCCUUUUUACN | 3500-3520 | 3500 |
| 103 | CUGUAAAAAGGCAACAGAGAG | 231 | CUCUCUGUUGCCUUUUUACAG | 3501-3521 | 3501 |
| 104 | UUGUAAAAAGGCAACAGAGAG | 232 | CUCUCUGUUGCCUUUUUACAA | 3501-3521 | 3501 |
| 105 | AUGUAAAAAGGCAACAGAGAG | 233 | CUCUCUGUUGCCUUUUUACAU | 3501-3521 | 3501 |
| 106 | NUGUAAAAAGGCAACAGAGAG | 234 | CUCUCUGUUGCCUUUUUACAN | 3501-3521 | 3501 |
| 107 | NUGUAAAAAGGCAACAGAGAN | 235 | NUCUCUGUUGCCUUUUUACAN | 3501-3521 | 3501 |
| 108 | GCUGUAAAAAGGCAACAGAGA | 236 | UCUCUGUUGCCUUUUUACAGC | 3502-3522 | 3502 |
| 109 | UCUGUAAAAAGGCAACAGAGA | 237 | UCUCUGUUGCCUUUUUACAGA | 3502-3522 | 3502 |
| 110 | ACUGUAAAAAGGCAACAGAGA | 238 | UCUCUGUUGCCUUUUUACAGU | 3502-3522 | 3502 |
| 111 | NCUGUAAAAAGGCAACAGAGA | 239 | UCUCUGUUGCCUUUUUACAGN | 3502-3522 | 3502 |
| 112 | NCUGUAAAAAGGCAACAGAGN | 240 | NCUCUGUUGCCUUUUUACAGN | 3502-3522 | 3502 |
| 113 | AAAACAGGUCUAGAAAAGUUG | 241 | CAACUUUUCUAGACCUGUUUU | 3523-3543 | 3523 |
| 114 | UAAACAGGUCUAGAAAAGUUG | 242 | CAACUUUUCUAGACCUGUUUA | 3523-3543 | 3523 |
| 115 | NAAACAGGUCUAGAAAAGUUG | 243 | CAACUUUUCUAGACCUGUUUN | 3523-3543 | 3523 |
| 116 | NAAACAGGUCUAGAAAAGUUN | 244 | NAACUUUUCUAGACCUGUUUN | 3523-3543 | 3523 |
| 117 | ACAAAAGCAAAACAGGUCUAG | 245 | CUAGACCUGUUUUGCUUUUGU | 3531-3551 | 3531 |
| 118 | UCAAAAGCAAAACAGGUCUAG | 246 | CUAGACCUGUUUUGCUUUUGA | 3531-3551 | 3531 |
| 119 | NCAAAAGCAAAACAGGUCUAG | 247 | CUAGACCUGUUUUGCUUUUGN | 3531-3551 | 3531 |
| 120 | NCAAAAGCAAAACAGGUCUAN | 248 | NUAGACCUGUUUUGCUUUUGN | 3531-3551 | 3531 |
| 121 | UACAAAAGCAAAACAGGUCUA | 249 | UAGACCUGUUUUGCUUUUGUA | 3532-3552 | 3532 |
| 122 | AACAAAAGCAAAACAGGUCUA | 250 | UAGACCUGUUUUGCUUUUGUU | 3532-3552 | 3532 |
| 123 | NACAAAAGCAAAACAGGUCUA | 251 | UAGACCUGUUUUGCUUUUGUN | 3532-3552 | 3532 |
| 124 | NACAAAAGCAAAACAGGUCUN | 252 | NAGACCUGUUUUGCUUUUGUN | 3532-3552 | 3532 |
| 125 | GUUACAAAAGCAAAACAGGUC | 253 | GACCUGUUUUGCUUUUGUAAC | 3534-3554 | 3534 |
| 126 | UUUACAAAAGCAAAACAGGUC | 254 | GACCUGUUUUGCUUUUGUAAA | 3534-3554 | 3534 |
| 127 | AUUACAAAAGCAAAACAGGUC | 255 | GACCUGUUUUGCUUUUGUAAU | 3534-3554 | 3534 |
| 128 | NUUACAAAAGCAAAACAGGUC | 256 | GACCUGUUUUGCUUUUGUAAN | 3534-3554 | 3534 |
| 129 | NUUACAAAAGCAAAACAGGUN | 257 | NACCUGUUUUGCUUUUGUAAN | 3534-3554 | 3534 |
| 130 | AAGUUACAAAAGCAAAACAGG | 258 | CCUGUUUUGCUUUUGUAACUU | 3536-3556 | 3536 |
| 131 | UAGUUACAAAAGCAAAACAGG | 259 | CCUGUUUUGCUUUUGUAACUA | 3536-3556 | 3536 |
| 132 | NAGUUACAAAAGCAAAACAGG | 260 | CCUGUUUUGCUUUUGUAACUN | 3536-3556 | 3536 |
| 133 | AAGUUACAAAAGCAAAACAGN | 261 | NCUGUUUUGCUUUUGUAACUU | 3536-3556 | 3536 |
| 134 | NAGUUACAAAAGCAAAACAGN | 262 | NCUGUUUUGCUUUUGUAACUN | 3536-3556 | 3536 |
| 135 | AAGUUACAAAAGCAAAACANN | 263 | NNUGUUUUGCUUUUGUAACUU | 3536-3556 | 3536 |
| 136 | UAGUUACAAAAGCAAAACANN | 264 | CCUGUUUUGCUUUUGUAACUA | 3536-3556 | 3536 |
| 137 | NAGUUACAAAAGCAAAACANN | 265 | NNUGUUUUGCUUUUGUAACUN | 3536-3556 | 3536 |
| 138 | AAGUUACAAAAGCAAAACNNN | 266 | NNNGUUUUGCUUUUGUAACUU | 3536-3556 | 3536 |
| 139 | UAGUUACAAAAGCAAAACNNN | 267 | NNNGUUUUGCUUUUGUAACUA | 3536-3556 | 3536 |
| 140 | NAGUUACAAAAGCAAAACNNN | 268 | NNNGUUUUGCUUUUGUAACUN | 3536-3556 | 3536 |
| 141 | CUUCAAGUUACAAAAGCAAAA | 269 | UUUUGCUUUUGUAACUUGAAG | 3540-3560 | 3540 |
| 142 | UUUCAAGUUACAAAAGCAAAA | 270 | UUUUGCUUUUGUAACUUGAAA | 3540-3560 | 3540 |
| 143 | AUUCAAGUUACAAAAGCAAAA | 271 | UUUUGCUUUUGUAACUUGAAU | 3540-3560 | 3540 |
| 144 | NUUCAAGUUACAAAAGCAAAA | 272 | UUUUGCUUUUGUAACUUGAAN | 3540-3560 | 3540 |
| 145 | NUUCAAGUUACAAAAGCAAAN | 273 | NUUUGCUUUUGUAACUUGAAN | 3540-3560 | 3540 |
| 146 | AAAUAUCUUCAAGUUACAAAA | 274 | UUUUGUAACUUGAAGAUAUUU | 3546-3566 | 3546 |
| 147 | UAAUAUCUUCAAGUUACAAAA | 275 | UUUUGUAACUUGAAGAUAUUA | 3546-3566 | 3546 |
| 148 | NAAUAUCUUCAAGUUACAAAA | 276 | UUUUGUAACUUGAAGAUAUUN | 3546-3566 | 3546 |
| 149 | NAAUAUCUUCAAGUUACAAAN | 277 | NUUUGUAACUUGAAGAUAUUN | 3546-3566 | 3546 |
| 150 | UAAAUAUCUUCAAGUUACAAA | 278 | UUUGUAACUUGAAGAUAUUUA | 3547-3567 | 3547 |
| 151 | AAAAUAUCUUCAAGUUACAAA | 279 | UUUGUAACUUGAAGAUAUUUU | 3547-3567 | 3547 |
| 152 | NAAAUAUCUUCAAGUUACAAA | 280 | UUUGUAACUUGAAGAUAUUUN | 3547-3567 | 3547 |
| 153 | NAAAUAUCUUCAAGUUACAAN | 281 | NUUGUAACUUGAAGAUAUUUN | 3547-3567 | 3547 |
| 154 | AUAAAUAUCUUCAAGUUACAA | 282 | UUGUAACUUGAAGAUAUUUAU | 3548-3568 | 3548 |
| 155 | UUAAAUAUCUUCAAGUUACAA | 283 | UUGUAACUUGAAGAUAUUUAA | 3548-3568 | 3548 |
| 156 | NUAAAUAUCUUCAAGUUACAA | 284 | UUGUAACUUGAAGAUAUUUAN | 3548-3568 | 3548 |
| 157 | NUAAAUAUCUUCAAGUUACAN | 285 | NUGUAACUUGAAGAUAUUUAN | 3548-3568 | 3548 |

The PCSK9 RNAi agent sense strands and antisense strands that comprise or consist of the sequences in Table 2 can be modified nucleotides or unmodified nucleotides. In some embodiments, the PCSK9 RNAi agents having the sense and antisense strand sequences that comprise or consist of the sequences in Table 2 are all or substantially all modified nucleotides.

In some embodiments, the antisense strand of a PCSK9 RNAi agent disclosed herein differs by 0, 1, 2, or 3 nucleotides from any of the antisense strand sequences in Table 2. In some embodiments, the sense strand of a PCSK9 RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified PCSK9 RNAi agent antisense strands, as well as their underlying unmodified nucleobase sequences, are provided in Table 3. Certain modified PCSK9 RNAi agent sense strands, as well as their underlying unmodified nucleobase sequences, are provided in Table 4. In forming PCSK9 RNAi agents, each of the nucleotides in each of the underlying base sequences listed in Tables 3 and 4, as well as in Table 2, above, can be a modified nucleotide.

The PCSK9 RNAi agents described herein are formed by annealing an antisense strand with a sense strand. A sense strand containing a sequence listed in Table 2 or Table 4, can be hybridized to any antisense strand containing a sequence listed in Table 2 or Table 3, provided the two sequences have a region of at least 85% complementarity over a contiguous 15, 16, 17, 18, 19, 20, or 21 nucleotide sequence.

In some embodiments, a PCSK9 RNAi agent antisense strand comprises a nucleotide sequence of any of the sequences in Table 2 or Table 3.

In some embodiments, a PCSK9 RNAi agent comprises or consists of a duplex having the nucleobase sequences of the sense strand and the antisense strand of any of the sequences in Table 2, Table 3 or Table 4. In some embodiments, a PCSK9 RNAi agent comprises or consists of a duplex sequence prepared or provided as a sodium salt, mixed salt, or a free-acid.

Examples of antisense strands containing modified nucleotides are provided in Table 3 and Table 5C. Examples of sense strands containing modified nucleotides are provided in Table 4 and Table 5C.

As used in Tables 3, 4, 5C, 7A, 7B, and 8, the following notations are used to indicate modified nucleotides and linking groups:
A=adenosine-3′-phosphate;
C=cytidine-3′-phosphate;
G=guanosine-3′-phosphate;
U=uridine-3′-phosphate
I=inosine-3′-phosphate
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
i=2′-O-methylinosine-3′-phosphate
is =2′-O-methylinosine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
A_{UNA}=2′,3′-seco-adenosine-3′-phosphate, see Table 6
A_{UNAS}=2′,3′-seco-adenosine-3′-phosphorothioate, see Table 6
C_{UNA}=2′,3′-seco-cytidine-3′-phosphate, see Table 6
C_{UNAS}=2′,3′-seco-cytidine-3′-phosphorothioate, see Table 6
G_{UNA}=2′,3′-seco-guanosine-3′-phosphate, see Table 6
G_{UNAS}=2′,3′-seco-guanosine-3′-phosphorothioate, see Table 6
U_{UNA}=2′,3′-seco-uridine-3′-phosphate, see Table 6
U_{UNAS}=2′,3′-seco-uridine-3′-phosphorothioate, see Table 6
a_2N=2′-O-methyl-2-aminoadenosine-3′-phosphate, see Table 6
a_2Ns=2′-O-methyl-2-aminoadenosine-3′-phosphorothioate, see Table 6
(invAb)=inverted abasic deoxyribonucleotide, see Table 6
(invAb)s=inverted abasic deoxyribonucleotide-5′-phosphorothioate, see Table 6
cPrpa=5′-cyclopropyl phosphonate-2′-O-methyladenosine-3′-phosphate (see Table 6)
cPrpas=5′-cyclopropyl phosphonate-2′-O-methyladenosine-3′-phosphorothioate (see Table 6)
cPrpu=5′-cyclopropyl phosphonate-2′-O-methyluridine-3′-phosphate (see Table 6)
cPrpus=5′-cyclopropyl phosphonate-2′-O-methyluridine-3′- phosphorothioate (see Table 6)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 6). Moreover, as the person of ordinary skill would readily understand and appreciate, while the phosphorothioate chemical structures depicted herein typically show the anion on the sulfur atom, the inventions disclosed herein encompass all phosphorothioate tautomers and resonance structures (e.g., where the sulfur atom has a double-bond and the anion is on an oxygen atom). Unless expressly indicated otherwise herein, such understandings of the person of ordinary skill in the art are used when describing the PCSK9 RNAi agents and compositions of PCSK9 RNAi agents disclosed herein.

Certain examples of targeting ligands, targeting groups, and linking groups used with the PCSK9 RNAi agents disclosed herein are provided below in Table 6. More specifically, targeting groups and linking groups (which together can form a targeting ligand) include (NAG37) and (NAG37)s, for which their chemical structures are provided below in Table 6. Each sense strand and/or antisense strand can have any targeting ligands, targeting groups, or linking groups listed herein, as well as other groups, conjugated to the 5′ and/or 3′ end of the sequence.

TABLE 3
PCSK9 RNAi Agent Antisense Strand Sequences

| | | | Underlying Base Sequence | |
|---|---|---|---|---|
| Antisense | | SEQ | (5′ → 3′) (Shown | SEQ |
| Strand | Modified Antisense Strand | ID | as an Unmodified | ID |
| ID: | (5′ → 3′) | NO. | Nucleotide Sequence) | NO. |
| AM10205-AS | asAfsgsAfcAfuGfcAfgGfaUfcUfuGfgUfsg | 286 | AAGACAUGCAGGAUCUUGGUG | 395 |
| AM10232-AS | asGfsasUfaAfaUfgUfcUfgCfuUfgCfuUfsg | 287 | AGAUAAAUGUCUGCUUGCUUG | 396 |
| AM10234-AS | usUfsgsUfaAfaAfaGfgCfaAfcAfgAfgAfsg | 288 | UUGUAAAAAGGCAACAGAGAG | 397 |
| AM10236-AS | usCfsusGfuAfaAfaAfgGfcAfaCfaGfaGfsa | 289 | UCUGUAAAAAGGCAACAGAGA | 398 |
| AM10238-AS | asCfsasAfaAfgCfaAfaAfcAfgGfuCfuAfsg | 290 | ACAAAAGCAAAACAGGUCUAG | 399 |
| AM10240-AS | usAfscsAfaAfaGfcAfaAfaCfaGfgUfcUfsa | 291 | UACAAAAGCAAAACAGGUCUA | 400 |
| AM10242-AS | asAfsgsUfuAfcAfaAfaGfcAfaAfaCfaGfsg | 292 | AAGUUACAAAAGCAAAACAGG | 401 |
| AM10702-AS | usGfsasUfcUfuGfgUfgAfgGfuAfuCfcCfsc | 293 | UGAUCUUGGUGAGGUAUCCCC | 402 |
| AM10704-AS | asGfsgsAfuCfuUfgGfuGfaGfgUfaUfcCfsc | 294 | AGGAUCUUGGUGAGGUAUCCC | 403 |
| AM10706-AS | usAfscsAfuGfC_{UNA}AfgGfaUfcUfuGfgUfgAfsg | 295 | UACAUGCAGGAUCUUGGUGAG | 404 |
| AM10728-AS | usAfsgsCfuUfcCfuGfgUfcUfgUfgUfuCfsc | 296 | UAGCUUCCUGGUCUGUGUUCC | 405 |
| AM10730-AS | asUfsasAfaUfgUfcUfgCfuUfgCfuUfgGfsg | 297 | AUAAAUGUCUGCUUGCUUGGG | 406 |
| AM10732-AS | usAfsasCfaGfaGfaGfgAfcAfgAfcCfcAfsa | 298 | UAACAGAGAGGACAGACCCAA | 407 |
| AM10734-AS | usGfsusAfaAfaAfgGfcAfaCfaGfaGfaGfsg | 299 | UGUAAAAAGGCAACAGAGAGG | 408 |
| AM10736-AS | usUfsusAfcAfaAfaGfcAfaAfaCfaGfgUfsc | 300 | UUUACAAAAGCAAAACAGGUC | 409 |
| AM10877-AS | usUfsuscaaguuacAfaAfaGfcaaasc | 301 | UUUCAAGUUACAAAAGCAAAC | 410 |
| AM11034-AS | asAfsgsUfuAfcAfaAfaGfcAfaAfaCfaGfsc | 302 | AAGUUACAAAAGCAAAACAGC | 411 |
| AM11036-AS | cPrpasAfsgsUfuAfcAfaAfaGfcAfaAfaCfaGfsg | 303 | AAGUUACAAAAGCAAAACAGG | 401 |
| AM11037-AS | asAfsgUfuAfcAfaAfaGfcAfaAfaCfasGfsg | 304 | AAGUUACAAAAGCAAAACAGG | 401 |
| AM11041-AS | asAfsgsuuacaaaaGfcAfaAfacagsg | 305 | AAGUUACAAAAGCAAAACAGG | 401 |
| AM11042-AS | asAfsGfsuuacaaaaGfcAfaAfacagsg | 306 | AAGUUACAAAAGCAAAACAGG | 401 |
| AM11295-AS | cPrpasAfsgsuuacaaaaGfcAfaAfacagsg | 307 | AAGUUACAAAAGCAAAACAGG | 401 |
| AM11296-AS | asAfsguuacaaaaGfcAfaAfacasgsg | 308 | AAGUUACAAAAGCAAAACAGG | 401 |
| AM11297-AS | cPrpasAfsguuacaaaaGfcAfaAfacasgsg | 309 | AAGUUACAAAAGCAAAACAGG | 401 |
| AM11987-AS | asAfsgsuuacaaaaGfcAfaAfacasgsg | 310 | AAGUUACAAAAGCAAAACAGG | 401 |
| AM11991-AS | usAfsgsuuacaaaaGfcAfaAfacasgsg | 311 | UAGUUACAAAAGCAAAACAGG | 412 |
| AM11992-AS | cPrpusAfsgsuuacaaaaGfcAfaAfacasgsg | 312 | UAGUUACAAAAGCAAAACAGG | 412 |
| AM11993-AS | cPrpusAfsguuacaaaaGfcAfaAfacasgsg | 313 | UAGUUACAAAAGCAAAACAGG | 412 |
| AM11994-AS | cPrpusAfguuacaaaaGfcAfaAfacasgsg | 314 | UAGUUACAAAAGCAAAACAGG | 412 |
| AM11995-AS | cPrpuAfguuacaaaaGfcAfaAfacasgsg | 315 | UAGUUACAAAAGCAAAACAGG | 412 |
| AM12085-AS | cPrpusUfsguaa_2NaaaggCfaAfcAfgagsasg | 316 | UUGUA(A^{2N})AAAGGCAACAGAGAG | 413 |
| AM12365-AS | asGfsucgcU_{UNA}ggagGfcAfcCfaausgsg | 317 | AGUCGCUGGAGGCACCAAUGG | 414 |
| AM12437-AS | cPrpusCfcaaaagauaAfaUfgUfcugcsu | 318 | UCCAAAAGAUAAAUGUCUGCU | 415 |
| AM12439-AS | asGfsagaggacagAfcCfcAfaaasgsg | 319 | AGAGAGGACAGACCCAAAAGG | 416 |
| AM12441-AS | asAfsasuaucuucaAfgUfuAfcaaasg | 320 | AAAUAUCUUCAAGUUACAAAG | 417 |
| AM12443-AS | cPrpusAfsasauaucuucAfaGfuUfacaasg | 321 | UAAAUAUCUUCAAGUUACAAG | 418 |
| AM12445-AS | cPrpusGfuagcA_{UNA}ggcaGfcAfcCfuggcsa | 322 | UGUAGCAGGCAGCACCUGGCA | 419 |
| AM12447-AS | asGfsgacagacccAfaAfaGfauasasg | 323 | AGGACAGACCCAAAAGAUAAG | 420 |
| AM12449-AS | cPrpusAfsasaugucugcUfuGfcUfugggsu | 324 | UAAAUGUCUGCUUGCUUGGGU | 421 |
| AM12451-AS | cPrpusCfsgaagU_{UNA}cgguGfaCfcAfugacsc | 325 | UCGAAGUCGGUGACCAUGACC | 422 |
| AM12453-AS | asAfsasacaggucuAfgAfaAfaguusg | 326 | AAAACAGGUCUAGAAAAGUUG | 423 |
| AM12455-AS | cPrpusGfsaugacaucuUfuGfgCfagasgsg | 327 | UGAUGACAUCUUUGGCAGAGG | 424 |
| AM12457-AS | cPrpusUfsusuccgaauaAfaCfuCfcaggsc | 328 | UUUUCCGAAUAAACUCCAGGC | 425 |
| AM12459-AS | asUfsasaauaucuuCfaAfgUfuacasg | 329 | AUAAAUAUCUUCAAGUUACAG | 426 |
| AM12718-AS | cPrpusUfsguaaaaaggCfaAfcAfgagsasg | 330 | UUGUAAAAAGGCAACAGAGAG | 397 |
| AM12719-AS | cPrpuUfguaaaaaggCfaAfcAfgagsasg | 331 | UUGUAAAAAGGCAACAGAGAG | 397 |
| AM12720-AS | cPrpuUfguaa_2NaaaggCfaAfcAfgagsasg | 332 | UUGUA(A^{2N})AAAGGCAACAGAGAG | 413 |
| AM12722-AS | cPrpuUfguaaaaaggCfaAfcAfgagasg | 333 | UUGUAAAAAGGCAACAGAGAG | 397 |
| AM12732-AS | cPrpuAfaauaucuucAfaGfuUfacaasg | 334 | UAAAUAUCUUCAAGUUACAAG | 418 |
| AM12734-AS | cPrpuAfaauaucuucAfaGfuUfacagsg | 335 | UAAAUAUCUUCAAGUUACAGG | 427 |
| AM12735-AS | cPrpusAfaauaucuucAfaGfuUfacagsg | 336 | UAAAUAUCUUCAAGUUACAGG | 427 |
| AM12737-AS | cPrpaGfagaggacagAfcCfcAfaaagsg | 337 | AGAGAGGACAGACCCAAAAGG | 416 |
| AM12739-AS | cPrpuGfagaggacagAfcCfcAfaaagsg | 338 | UGAGAGGACAGACCCAAAAGG | 428 |
| AM12759-AS | cPrpaAfaacaggucuAfgAfaAfaguusg | 339 | AAAACAGGUCUAGAAAAGUUG | 423 |
| AM12878-AS | cPrpaAfguuacaaaaGfcAfaAfacagsg | 340 | AAGUUACAAAAGCAAAACAGG | 401 |
| AM13565-AS | asGfsuuaCfaaaagcaAfaAfcagsgsu | 341 | AGUUACAAAAGCAAAACAGGU | 429 |
| AM14058-AS | cPrpasAfsgUfuacaaaaGfcAfaAfacasgsg | 342 | AAGUUACAAAAGCAAAACAGG | 401 |
| AM14059-AS | cPrpasAfsguuAfcaaaaGfcAfaAfacasgsg | 343 | AAGUUACAAAAGCAAAACAGG | 401 |
| AM14060-AS | cPrpasAfsguuacAfaaaGfcAfaAfacasgsg | 344 | AAGUUACAAAAGCAAAACAGG | 401 |
| AM14061-AS | cPrpasAfsguuacaaAfaGfcAfaAfacasgsg | 345 | AAGUUACAAAAGCAAAACAGG | 401 |
| AM14062-AS | cPrpasAfsguuacaaaaGfcAfaAfaCfasgsg | 346 | AAGUUACAAAAGCAAAACAGG | 401 |

(A^{2N})= 2-aminoadenosine nucleotide

TABLE 4
PCSK9 RNAi Agent Sense Strand Sequences

| | | | Underlying Base Sequence | |
|---|---|---|---|---|
| Sense | | SEQ | (5′ → 3′) (Shown | SEQ |
| Strand | | ID | as an Unmodified | ID |
| ID: | Modified Sense Strand (5′ → 3′) | NO. | Nucleotide Sequence) | NO. |
| AM11040-SS | (NAG37)s(invAb)sccuguuuuGfcUfuUfuguaacuus(invAb) | 368 | CCUGUUUUGCUUUUGUAACUU | 430 |
| AM10204-SS | (NAG37)s(invAb)scaccaagaUfCfCfugcaugucuus(invAb) | 348 | CACCAAGAUCCUGCAUGUCUU | 431 |
| AM10231-SS | (NAG37)s(invAb)sca_2NagcaagCfAfGfacauuuaucus(invAb) | 349 | C(A^{2N})AGCAAGCAGACAUUUAUCU | 432 |
| AM10233-SS | (NAG37)s(invAb)scucucuguUfGfCfcuuuuuacaas(invAb) | 350 | CUCUCUGUUGCCUUUUUACAA | 433 |
| AM10235-SS | (NAG37)s(invAb)sucucuguuGfCfCfuuuuuacagas(invAb) | 351 | UCUCUGUUGCCUUUUUACAGA | 434 |
| AM10237-SS | (NAG37)s(invAb)scuagaccuGfUfUfuugcuuuugus(invAb) | 352 | CUAGACCUGUUUUGCUUUUGU | 435 |
| AM10239-SS | (NAG37)s(invAb)sua_2NgaccugUfUfUfugcuuuuguas(invAb) | 353 | U(A^{2N})GACCUGUUUUGCUUUUGUA | 436 |
| AM10241-SS | (NAG37)s(invAb)sccuguuuuGfCfUfuuuguaacuus(invAb) | 354 | CCUGUUUUGCUUUUGUAACUU | 430 |
| AM10701-SS | (NAG37)s(invAb)sggggauacCfUfCfaccaaiaucas(invAb) | 355 | GGGGAUACCUCACCAAIAUCA | 437 |
| AM10703-SS | (NAG37)s(invAb)sgggauaccUfCfAfccaaiauccus(invAb) | 356 | GGGAUACCUCACCAAIAUCCU | 438 |
| AM10705-SS | (NAG37)s(invAb)scucaccaaGfAfUfccugcauguas(invAb) | 357 | CUCACCAAGAUCCUGCAUGUA | 439 |
| AM10727-SS | (NAG37)s(invAb)sggaacacaGfAfCfcaggaaicuas(invAb) | 358 | GGAACACAGACCAGGAAICUA | 440 |
| AM10729-SS | (NAG37)s(invAb)scccaagcaAfGfCfagacauuua_2Nus(invAb) | 359 | CCCAAGCAAGCAGACAUUU(A^{2N})U | 441 |
| AM10731-SS | (NAG37)s(invAb)suugggucuGfUfCfcucucuguuas(invAb) | 360 | UUGGGUCUGUCCUCUCUGUUA | 442 |
| AM10733-SS | (NAG37)s(invAb)sccucucugUfUfGfccuuuuuacas(invAb) | 361 | CCUCUCUGUUGCCUUUUUACA | 443 |
| AM10735-SS | (NAG37)s(invAb)sgaccuguuUfUfGfcuuuuguaa_2Nas(invAb) | 362 | GACCUGUUUUGCUUUUGUA(A^{2N})A | 444 |
| AM10876-SS | (NAG37)s(invAb)sguuugcUfuUfuGfuaacuugaaas(invAb) | 363 | GUUUGCUUUUGUAACUUGAAA | 445 |
| AM11033-SS | (NAG37)s(invAb)sgcuguuuuGfCfUfuuuguaacuus(invAb) | 364 | GCUGUUUUGCUUUUGUAACUU | 446 |
| AM11035-SS | (NAG37)s(invAb)sccuguuuuGfCfUfuuugua_2Nacuus(invAb) | 365 | CCUGUUUUGCUUUUGU(A^{2N})ACUU | 447 |
| AM11038-SS | (NAG37)s(invAb)sccuguuUfuGfcUfuuuguaacuus(invAb) | 366 | CCUGUUUUGCUUUUGUAACUU | 430 |
| AM11039-SS | (NAG37)s(invAb)sccuguuuuGfcUfUfuuguaacuus(invAb) | 367 | CCUGUUUUGCUUUUGUAACUU | 430 |
| AM11040-SS | (NAG37)s(invAb)sccuguuuuGfcUfuUfuguaacuus(invAb) | 368 | CCUGUUUUGCUUUUGUAACUU | 430 |
| AM11294-SS | (NAG37)s(invAb)sccuguuuuGfcUfuUfugua_2Nacuus(invAb) | 369 | CCUGUUUUGCUUUUGU(A^{2N})ACUU | 447 |
| AM11988-SS | (NAG37)s(invAb)sccuguuuuGfcUfUfuugua_2Nacuus(invAb) | 370 | CCUGUUUUGCUUUUGU(A^{2N})ACUU | 447 |
| AM11989-SS | (NAG37)s(invAb)sccuguuuuGfcUfuuugua_2Nacuus(invAb) | 371 | CCUGUUUUGCUUUUGU(A^{2N})ACUU | 447 |
| AM11990-SS | (NAG37)s(invAb)sccuguuuuGfCfUfuuugua_2Nacuas(invAb) | 372 | CCUGUUUUGCUUUUGU(A^{2N})ACUA | 448 |
| AM12084-SS | (NAG37)s(invAb)scucucuguUfgCfcUfuuuuacaas(invAb) | 373 | CUCUCUGUUGCCUUUUUACAA | 433 |
| AM12364-SS | (NAG37)s(invAb)sccauugguGfcCfuCfcagcgacus(invAb) | 374 | CCAUUGGUGCCUCCAGCGACU | 449 |
| AM12436-SS | (NAG37)s(invAb)sagcagacaUfUfUfaucuuuuigas(invAb) | 375 | AGCAGACAUUUAUCUUUUIGA | 450 |
| AM12438-SS | (NAG37)s(invAb)sccuuuuggGfUfCfuguccucucus(invAb) | 376 | CCUUUUGGGUCUGUCCUCUCU | 451 |
| AM12440-SS | (NAG37)s(invAb)scuuuguaaCfUfUfgaagauauuus(invAb) | 377 | CUUUGUAACUUGAAGAUAUUU | 452 |
| AM12442-SS | (NAG37)s(invAb)scuuguaacUfUfGfaagauauuuas(invAb) | 378 | CUUGUAACUUGAAGAUAUUUA | 453 |
| AM12444-SS | (NAG37)s(invAb)sugccagguGfCfUfgccugcuacas(invAb) | 379 | UGCCAGGUGCUGCCUGCUACA | 454 |
| AM12446-SS | (NAG37)s(invAb)scuuaucuuUfUfGfggucuiuccus(invAb) | 380 | CUUAUCUUUUGGGUCUIUCCU | 455 |
| AM12448-SS | (NAG37)s(invAb)sacccaagcAfAfGfcagacauuuas(invAb) | 381 | ACCCAAGCAAGCAGACAUUUA | 456 |
| AM12450-SS | (NAG37)s(invAb)sggucauggUfCfAfccgacuucgas(invAb) | 382 | GGUCAUGGUCACCGACUUCGA | 457 |
| AM12452-SS | (NAG37)s(invAb)scaacuuuuCfUfAfgaccuguuuus(invAb) | 383 | CAACUUUUCUAGACCUGUUUU | 458 |
| AM12454-SS | (NAG37)s(invAb)sccucugccAfAfAfgaugucaucas(invAb) | 384 | CCUCUGCCAAAGAUGUCAUCA | 459 |
| AM12456-SS | (NAG37)s(invAb)sgccuggagUfUfUfauucggaaa_2Nas(invAb) | 385 | GCCUGGAGUUUAUUCGGAA(A^{2N})A | 460 |
| AM12458-SS | (NAG37)s(invAb)scuguaacuUfGfAfagauauuua_2Nus(invAb) | 386 | CUGUAACUUGAAGAUAUUU(A^{2N})U | 461 |
| AM12506-SS | (NAG37)s(invAb)sccuguuuuGfcUfUfuugua_2Nacuas(invAb) | 387 | CCUGUUUUGCUUUUGU(A^{2N})ACUA | 448 |
| AM12507-SS | (NAG37)s(invAb)sccuguuuuGfcUfUfuuguaacuas(invAb) | 388 | CCUGUUUUGCUUUUGUAACUA | 462 |
| AM12508-SS | (NAG37)s(invAb)sccuguuuuGfCfUfuuuguaacuas(invAb) | 389 | CCUGUUUUGCUUUUGUAACUA | 462 |
| AM12721-SS | (NAG37)s(invAb)scucucuguUfgCfCfuuuuuacaas(invAb) | 390 | CUCUCUGUUGCCUUUUUACAA | 433 |
| AM12723-SS | (NAG37)s(invAb)scucucuguuGfCfcuuuuuacaas(invAb) | 391 | CUCUCUGUUGCCUUUUUACAA | 433 |
| AM12733-SS | (NAG37)s(invAb)sccuguaacUfUfGfaagauauuuas(invAb) | 392 | CCUGUAACUUGAAGAUAUUUA | 463 |
| AM12736-SS | (NAG37)s(invAb)sccuguaacUfUfGfaagaua_2Nuuuas(invAb) | 393 | CCUGUAACUUGAAGAU(A^{2N})UUUA | 464 |
| AM12738-SS | (NAG37)s(invAb)sccuuuuggGfUfCfuguccucucas(invAb) | 394 | CCUUUUGGGUCUGUCCUCUCA | 465 |

(A^{2N})= 2-aminoadenosine nucleotide;
i= hypoxanthine (inosine) nucleotide

The PCSK9 RNAi agents described herein are formed by annealing an antisense strand with a sense strand. A sense strand containing a sequence listed in Table 2, Table 4, Table 5C, Table 7B, or Table 8 can be hybridized to any antisense strand containing a sequence listed in Table 2, Table 3, Table 5C, Table 7A, or Table 8 provided the two sequences have a region of at least 85% complementarity over a contiguous 15, 16, 17, 18, 19, 20, or 21 nucleotide sequence.

In some embodiments, the antisense strand of a PCSK9 RNAi agent disclosed herein differs by 0, 1, 2, or 3 nucleotides from any of the antisense strand sequences in Table 3 or Table 5C. In some embodiments, the sense strand of a PCSK9 RNAi agent disclosed herein differs by 0, 1, 2, or 3 nucleotides from any of the sense strand sequences in Table 4 or Table 5C.

In some embodiments, a PCSK9 RNAi agent antisense strand comprises a nucleotide sequence of any of the sequences in Table 2, Table 3, or Table 5C. In some embodiments, a PCSK9 RNAi agent antisense strand comprises the sequence of nucleotides (from 5′ end→3′ end) 1-17, 2-17, 1-18, 2-18, 1-19, 2-19, 1-20, 2-20, 1-21, or 2-21, of any of the sequences in Table 2, Table 3, or Table 5C. In certain embodiments, a PCSK9 RNAi agent antisense strand comprises or consists of a modified sequence of any one of the modified sequences in Table 3 or Table 5C.

In some embodiments, a PCSK9 RNAi agent sense strand comprises the nucleotide sequence of any of the sequences in Table 2, Table 4, or Table 5C. In some embodiments, a PCSK9 RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, or 4-21, of any of the sequences in Table 2, Table 4, or Table 5C. In certain embodiments, a PCSK9 RNAi agent sense strand comprises or consists of a modified sequence of any one of the modified sequences in Table 4 or Table 5C.

For the PCSK9 RNAi agents disclosed herein, the nucleotide at position 1 of the antisense strand (from 5′ end→3′ end) can be perfectly complementary to a PCSK9 gene, or can be non-complementary to a PCSK9 gene. In some embodiments, the nucleotide at position 1 of the antisense strand (from 5′ end→3′ end) is a U, A, or dT (or a modified version thereof). In some embodiments, the nucleotide at position 1 of the antisense strand (from 5′ end→3′ end) forms an A:U or U:A base pair with the sense strand.

A sense strand containing a sequence listed in Table 2, Table 4, Table 5C, Table 7B, or Table 8 can be hybridized to any antisense strand containing a sequence listed in Table 2, Table 3, Table 5C, Table 7A, or Table 8 provided the two sequences have a region of at least 85% complementarity over a contiguous 15, 16, 17, 18, 19, 20, or 21 nucleotide sequence. In some embodiments, the PCSK9 RNAi agent has a sense strand consisting of the modified sequence of any of the modified sequences in Table 4 or Table 5C, and an antisense strand consisting of the modified sequence of any of the modified sequences in Table 3 or Table 5C. Certain representative sequence pairings are exemplified by the Duplex ID Nos. shown in Tables 5A, 5B, 5C., and 8.

In some embodiments, a PCSK9 RNAi agent comprises, consists of, or consists essentially of a duplex represented by any one of the Duplex ID Nos. presented herein. In some embodiments, a PCSK9 RNAi agent comprises the sense strand and antisense strand nucleotide sequences of any of the duplexes represented by any of the Duplex ID Nos. presented herein. In some embodiments, a PCSK9 RNAi agent comprises the sense strand and antisense strand nucleotide sequences of any of the duplexes represented by any of the Duplex ID Nos. presented herein and a targeting group and/or linking group wherein the targeting group and/or linking group is covalently linked (i.e., conjugated) to the sense strand or the antisense strand. In some embodiments, a PCSK9 RNAi agent includes the sense strand and antisense strand modified nucleotide sequences of any of the Duplex ID Nos. presented herein. In some embodiments, a PCSK9 RNAi agent comprises the sense strand and antisense strand modified nucleotide sequences of any of the Duplex ID Nos. presented herein and a targeting group and/or linking group, wherein the targeting group and/or linking group is covalently linked to the sense strand or the antisense strand.

In some embodiments, a PCSK9 RNAi agent comprises an antisense strand and a sense strand having the nucleotide sequences of any of the antisense strand/sense strand duplexes of Table 2 or Tables 5A, 5B, and 5C, and further comprises a targeting group or targeting ligand. In some embodiments, a PCSK9 RNAi agent comprises an antisense strand and a sense strand having the nucleotide sequences of any of the antisense strand/sense strand duplexes of Table 2 or Tables 5A, 5B, and 5C, and further comprises an asialoglycoprotein receptor ligand targeting group.

A targeting group, with or without a linker, can be linked to the 5′ or 3′ end of any of the sense and/or antisense strands disclosed in Tables 2, 3, 4, or 5C. A linker, with or without a targeting group, can be attached to the 5′ or 3′ end of any of the sense and/or antisense strands disclosed in Tables 2, 3, 4, and 5C.

In some embodiments, a PCSK9 RNAi agent comprises an antisense strand and a sense strand having the nucleotide sequences of any of the antisense strand/sense strand duplexes of Table 2 or Tables 5A, 5B and 5C, and further comprises a targeting ligand selected from the group consisting of. (NAG37) and (NAG37)s, each as defined in Table 6.

In some embodiments, a PCSK9 RNAi agent comprises an antisense strand and a sense strand having the modified nucleotide sequence of any of the antisense strand and/or sense strand nucleotide sequences in Table 3 or Table 4.

In some embodiments, a PCSK9 RNAi agent comprises an antisense strand and a sense strand having a modified nucleotide sequence of any of the antisense strand and/or sense strand nucleotide sequences of any of the duplexes Tables 5A, 5B, and 5C, and further comprises an asialoglycoprotein receptor ligand targeting group.

In some embodiments, a PCSK9 RNAi agent comprises, consists of, or consists essentially of any of the duplexes of Tables 5A, 5B, and 5C.

TABLE 5A
PCSK9 RNAi Agents Duplexes with Corresponding Sense and Antisense Strand ID Numbers
and Sequence ID numbers for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD07404 | AM10205-AS | 286 | 395 | AM10204-SS | 348 | 431 |
| AD07418 | AM10232-AS | 287 | 396 | AM10231-SS | 349 | 432 |
| AD07419 | AM10234-AS | 288 | 397 | AM10233-SS | 350 | 433 |
| AD07420 | AM10236-AS | 289 | 398 | AM10235-SS | 351 | 434 |
| AD07421 | AM10238-AS | 290 | 399 | AM10237-SS | 352 | 435 |
| AD07422 | AM10240-AS | 291 | 400 | AM10239-SS | 353 | 436 |
| AD07423 | AM10242-AS | 292 | 401 | AM10241-SS | 354 | 430 |
| AD07690 | AM10702-AS | 293 | 402 | AM10701-SS | 355 | 437 |
| AD07691 | AM10704-AS | 294 | 403 | AM10703-SS | 356 | 438 |
| AD07692 | AM10706-AS | 295 | 404 | AM10705-SS | 357 | 439 |
| AD07710 | AM10728-AS | 296 | 405 | AM10727-SS | 358 | 440 |
| AD07711 | AM10730-AS | 297 | 406 | AM10729-SS | 359 | 441 |
| AD07712 | AM10732-AS | 298 | 407 | AM10731-SS | 360 | 442 |
| AD07713 | AM10734-AS | 299 | 408 | AM10733-SS | 361 | 443 |
| AD07714 | AM10736-AS | 300 | 409 | AM10735-SS | 362 | 444 |
| AD07802 | AM10877-AS | 301 | 410 | AM10876-SS | 363 | 445 |
| AD07911 | AM11034-AS | 302 | 411 | AM11033-SS | 364 | 446 |
| AD07912 | AM10242-AS | 292 | 401 | AM11035-SS | 365 | 447 |
| AD07913 | AM11036-AS | 303 | 401 | AM10241-SS | 354 | 430 |
| AD07914 | AM11037-AS | 304 | 401 | AM10241-SS | 354 | 430 |
| AD07915 | AM10242-AS | 292 | 401 | AM11038-SS | 366 | 430 |
| AD07916 | AM10242-AS | 292 | 401 | AM11039-SS | 367 | 430 |
| AD07917 | AM10242-AS | 292 | 401 | AM11040-SS | 368 | 430 |
| AD07918 | AM11041-AS | 305 | 401 | AM10241-SS | 354 | 430 |
| AD07919 | AM11042-AS | 306 | 401 | AM10241-SS | 354 | 430 |
| AD08105 | AM10242-AS | 292 | 401 | AM11294-SS | 369 | 447 |
| AD08106 | AM11041-AS | 305 | 401 | AM11035-SS | 365 | 447 |
| AD08107 | AM11041-AS | 305 | 401 | AM11294-SS | 369 | 447 |
| AD08108 | AM11295-AS | 307 | 401 | AM11294-SS | 369 | 447 |
| AD08109 | AM11296-AS | 308 | 401 | AM11294-SS | 369 | 447 |
| AD08110 | AM11297-AS | 309 | 401 | AM11294-SS | 369 | 447 |
| AD08574 | AM11987-AS | 310 | 401 | AM11035-SS | 365 | 447 |
| AD08575 | AM11987-AS | 310 | 401 | AM11988-SS | 370 | 447 |
| AD08576 | AM11987-AS | 310 | 401 | AM11989-SS | 371 | 447 |
| AD08577 | AM11991-AS | 311 | 412 | AM11990-SS | 372 | 448 |
| AD08578 | AM11992-AS | 312 | 412 | AM11990-SS | 372 | 448 |
| AD08579 | AM11993-AS | 313 | 412 | AM11990-SS | 372 | 448 |
| AD08580 | AM11994-AS | 314 | 412 | AM11990-SS | 372 | 448 |
| AD08581 | AM11995-AS | 315 | 412 | AM11990-SS | 372 | 448 |
| AD08631 | AM12085-AS | 316 | 413 | AM12084-SS | 373 | 433 |
| AD08796 | AM12365-AS | 317 | 414 | AM12364-SS | 374 | 449 |
| AD08834 | AM12437-AS | 318 | 415 | AM12436-SS | 375 | 450 |
| AD08835 | AM12439-AS | 319 | 416 | AM12438-SS | 376 | 451 |
| AD08836 | AM12441-AS | 320 | 417 | AM12440-SS | 377 | 452 |
| AD08837 | AM12443-AS | 321 | 418 | AM12442-SS | 378 | 453 |
| AD08838 | AM12445-AS | 322 | 419 | AM12444-SS | 379 | 454 |
| AD08839 | AM12447-AS | 323 | 420 | AM12446-SS | 380 | 455 |
| AD08840 | AM12449-AS | 324 | 421 | AM12448-SS | 381 | 456 |
| AD08841 | AM12451-AS | 325 | 422 | AM12450-SS | 382 | 457 |
| AD08842 | AM12453-AS | 326 | 423 | AM12452-SS | 383 | 458 |
| AD08843 | AM12455-AS | 327 | 424 | AM12454-SS | 384 | 459 |
| AD08844 | AM12457-AS | 328 | 425 | AM12456-SS | 385 | 460 |
| AD08845 | AM12459-AS | 329 | 426 | AM12458-SS | 386 | 461 |
| AD08878 | AM11995-AS | 315 | 412 | AM12506-SS | 387 | 448 |
| AD08879 | AM11995-AS | 315 | 412 | AM12507-SS | 388 | 462 |
| AD08880 | AM11995-AS | 315 | 412 | AM12508-SS | 389 | 462 |
| AD09027 | AM12085-AS | 316 | 413 | AM10233-SS | 350 | 433 |
| AD09028 | AM12718-AS | 330 | 397 | AM10233-SS | 350 | 433 |
| AD09029 | AM12719-AS | 331 | 397 | AM10233-SS | 350 | 433 |
| AD09030 | AM12720-AS | 332 | 413 | AM10233-SS | 350 | 433 |
| AD09031 | AM12720-AS | 332 | 413 | AM12721-SS | 390 | 433 |
| AD09032 | AM12719-AS | 331 | 397 | AM12721-SS | 390 | 433 |
| AD09033 | AM12722-AS | 333 | 397 | AM12721-SS | 390 | 433 |
| AD09034 | AM12722-AS | 333 | 397 | AM12723-SS | 391 | 433 |
| AD09038 | AM12732-AS | 334 | 418 | AM12442-SS | 378 | 453 |
| AD09039 | AM12734-AS | 335 | 427 | AM12733-SS | 392 | 463 |
| AD09040 | AM12735-AS | 336 | 427 | AM12733-SS | 392 | 463 |
| AD09041 | AM12735-AS | 336 | 427 | AM12736-SS | 393 | 464 |
| AD09042 | AM12737-AS | 337 | 416 | AM12438-SS | 376 | 451 |
| AD09043 | AM12739-AS | 338 | 428 | AM12738-SS | 394 | 465 |
| AD09059 | AM12759-AS | 339 | 423 | AM12452-SS | 383 | 458 |
| AD09131 | AM11295-AS | 307 | 401 | AM10241-SS | 354 | 430 |
| AD09132 | AM12878-AS | 340 | 401 | AM10241-SS | 354 | 430 |
| AD09327 | AM11297-AS | 309 | 401 | AM11040-SS | 368 | 430 |
| AD09851 | AM14058-AS | 342 | 401 | AM11040-SS | 368 | 430 |
| AD09852 | AM14059-AS | 343 | 401 | AM11040-SS | 368 | 430 |
| AD09853 | AM14060-AS | 344 | 401 | AM11040-SS | 368 | 430 |
| AD09854 | AM14061-AS | 345 | 401 | AM11040-SS | 368 | 430 |
| AD09855 | AM14062-AS | 346 | 401 | AM11040-SS | 368 | 430 |

TABLE 5B
PCSK9 RNAi Agents Duplexes with Corresponding Sense
and Antisense Strand ID Numbers Referencing Position
Targeted on PCSK9 Gene (SEQ ID NO: 1)

| | | | Targeted PCSK9 Gene |
|---|---|---|---|
| | Antisense | | Position |
| Duplex ID | Strand ID | Sense Strand ID | (Of SEQ ID NO: 1) |
| AD07404 | AM10205-AS | AM10204-SS | 614 |
| AD07418 | AM10232-AS | AM10231-SS | 3468 |
| AD07419 | AM10234-AS | AM10233-SS | 3501 |
| AD07420 | AM10236-AS | AM10235-SS | 3502 |
| AD07421 | AM10238-AS | AM10237-SS | 3531 |
| AD07422 | AM10240-AS | AM10239-SS | 3532 |
| AD07423 | AM10242-AS | AM10241-SS | 3536 |
| AD07690 | AM10702-AS | AM10701-SS | 604 |
| AD07691 | AM10704-AS | AM10703-SS | 605 |
| AD07692 | AM10706-AS | AM10705-SS | 612 |
| AD07710 | AM10728-AS | AM10727-SS | 3301 |
| AD07711 | AM10730-AS | AM10729-SS | 3466 |
| AD07712 | AM10732-AS | AM10731-SS | 3466 |
| AD07713 | AM10734-AS | AM10733-SS | 3500 |
| AD07714 | AM10736-AS | AM10735-SS | 3534 |
| AD07802 | AM10877-AS | AM10876-SS | 3540 |
| AD07911 | AM11034-AS | AM11033-SS | 3536 |
| AD07912 | AM10242-AS | AM11035-SS | 3536 |
| AD07913 | AM11036-AS | AM10241-SS | 3536 |
| AD07914 | AM11037-AS | AM10241-SS | 3536 |
| AD07915 | AM10242-AS | AM11038-SS | 3536 |
| AD07916 | AM10242-AS | AM11039-SS | 3536 |
| AD07917 | AM10242-AS | AM11040-SS | 3536 |
| AD07918 | AM11041-AS | AM10241-SS | 3536 |
| AD07919 | AM11042-AS | AM10241-SS | 3536 |
| AD08105 | AM10242-AS | AM11294-SS | 3536 |
| AD08106 | AM11041-AS | AM11035-SS | 3536 |
| AD08107 | AM11041-AS | AM11294-SS | 3536 |
| AD08108 | AM11295-AS | AM11294-SS | 3536 |
| AD08109 | AM11296-AS | AM11294-SS | 3536 |
| AD08110 | AM11297-AS | AM11294-SS | 3536 |
| AD08574 | AM11987-AS | AM11035-SS | 3536 |
| AD08575 | AM11987-AS | AM11988-SS | 3536 |
| AD08576 | AM11987-AS | AM11989-SS | 3536 |
| AD08577 | AM11991-AS | AM11990-SS | 3536 |
| AD08578 | AM11992-AS | AM11990-SS | 3536 |
| AD08579 | AM11993-AS | AM11990-SS | 3536 |
| AD08580 | AM11994-AS | AM11990-SS | 3536 |
| AD08581 | AM11995-AS | AM11990-SS | 3536 |
| AD08631 | AM12085-AS | AM12084-SS | 3501 |
| AD08796 | AM12365-AS | AM12364-SS | 1393 |
| AD08834 | AM12437-AS | AM12436-SS | 3474 |
| AD08835 | AM12439-AS | AM12438-SS | 3486 |
| AD08836 | AM12441-AS | AM12440-SS | 3546 |
| AD08837 | AM12443-AS | AM12442-SS | 3547 |
| AD08838 | AM12445-AS | AM12444-SS | 1859 |
| AD08839 | AM12447-AS | AM12446-SS | 3482 |
| AD08840 | AM12449-AS | AM12448-SS | 3465 |
| AD08841 | AM12451-AS | AM12450-SS | 887 |
| AD08842 | AM12453-AS | AM12452-SS | 3523 |
| AD08843 | AM12455-AS | AM12454-SS | 1543 |
| AD08844 | AM12457-AS | AM12456-SS | 1090 |
| AD08845 | AM12459-AS | AM12458-SS | 3548 |
| AD08878 | AM11995-AS | AM12506-SS | 3536 |
| AD08879 | AM11995-AS | AM12507-SS | 3536 |
| AD08880 | AM11995-AS | AM12508-SS | 3536 |
| AD09027 | AM12085-AS | AM10233-SS | 3501 |
| AD09028 | AM12718-AS | AM10233-SS | 3501 |
| AD09029 | AM12719-AS | AM10233-SS | 3501 |
| AD09030 | AM12720-AS | AM10233-SS | 3501 |
| AD09031 | AM12720-AS | AM12721-SS | 3501 |
| AD09032 | AM12719-AS | AM12721-SS | 3501 |
| AD09033 | AM12722-AS | AM12721-SS | 3501 |
| AD09034 | AM12722-AS | AM12723-SS | 3501 |
| AD09038 | AM12732-AS | AM12442-SS | 3547 |
| AD09039 | AM12734-AS | AM12733-SS | 3547 |
| AD09040 | AM12735-AS | AM12733-SS | 3547 |
| AD09041 | AM12735-AS | AM12736-SS | 3547 |
| AD09042 | AM12737-AS | AM12438-SS | 3486 |
| AD09043 | AM12739-AS | AM12738-SS | 3486 |
| AD09059 | AM12759-AS | AM12452-SS | 3523 |
| AD09131 | AM11295-AS | AM10241-SS | 3536 |
| AD09132 | AM12878-AS | AM10241-SS | 3536 |
| AD09327 | AM11297-AS | AM11040-SS | 3536 |
| AD09851 | AM14058-AS | AM11040-SS | 3536 |
| AD09852 | AM14059-AS | AM11040-SS | 3536 |
| AD09853 | AM14060-AS | AM11040-SS | 3536 |
| AD09854 | AM14061-AS | AM11040-SS | 3536 |
| AD09855 | AM14062-AS | AM11040-SS | 3536 |

TABLE 5C
PCSK9 RNAi Agent Duplexes Showing Chemically Modified Antisense Strand and Sense Strand Sequences

| | | SEQ | | SEQ |
|---|---|---|---|---|
| Duplex | | ID | | ID |
| ID: | Modified Antisense Strand (5′ → 3′) | NO. | Modified Sense Strand (5′ → 3′) | NO. |
| AD07404 | asAfsgsAfcAfuGfcAfgGfaUfcUfuGfgUfsg | 286 | (NAG37)s(invAb)scaccaagaUfCfCfugcaugucuus(invAb) | 348 |
| AD07418 | asGfsasUfaAfaUfgUfcUfgCfuUfgCfuUfsg | 287 | (NAG37)s(invAb)sca_2NagcaagCfAfGfacauuuaucus | 349 |
| | | | (invAb) | |
| AD07419 | usUfsgsUfaAfaAfaGfgCfaAfcAfgAfgAfsg | 288 | (NAG37)s(invAb)scucucuguUfGfCfcuuuuuacaas(invAb) | 350 |
| AD07420 | usCfsusGfuAfaAfaAfgGfcAfaCfaGfaGfsa | 289 | (NAG37)s(invAb)sucucuguuGfCfCfuuuuuacagas(invAb) | 351 |
| AD07421 | asCfsasAfaAfgCfaAfaAfcAfgGfuCfuAfsg | 290 | (NAG37)s(invAb)scuagaccuGfUfUfuugcuuuugus(invAb) | 352 |
| AD07422 | usAfscsAfaAfaGfcAfaAfaCfaGfgUfcUfsa | 291 | (NAG37)s(invAb)sua_2NgaccugUfUfUfugcuuuuguas | 353 |
| | | | (invAb) | |
| AD07423 | asAfsgsUfuAfcAfaAfaGfcAfaAfaCfaGfsg | 292 | (NAG37)s(invAb)sccuguuuuGfCfUfuuuguaacuus(invAb) | 354 |
| AD07690 | usGfsasUfcUfuGfgUfgAfgGfuAfuCfcCfsc | 293 | (NAG37)s(invAb)sggggauacCfUfCfaccaaiaucas(invAb) | 355 |
| AD07691 | asGfsgsAfuCfuUfgGfuGfaGfgUfaUfcCfsc | 294 | (NAG37)s(invAb)sgggauaccUfCfAfccaaiauccus(invAb) | 356 |
| AD07692 | usAfscsAfuGfC_{UNA}AfgGfaUfcUfuGfgUfgAf | 295 | (NAG37)s(invAb)scucaccaaGfAfUfccugcauguas(invAb) | 357 |
| | sg | | | |
| AD07710 | usAfsgsCfuUfcCfuGfgUfcUfgUfgUfuCfsc | 296 | (NAG37)s(invAb)sggaacacaGfAfCfcaggaaicuas(invAb) | 358 |
| AD07711 | asUfsasAfaUfgUfcUfgCfuUfgCfuUfgGfsg | 297 | (NAG37)s(invAb)scccaagcaAfGfCfagacauuua_2Nus | 359 |
| | | | (invAb) | |
| AD07712 | usAfsasCfaGfaGfaGfgAfcAfgAfcCfcAfsa | 298 | (NAG37)s(invAb)suugggucuGfUfCfcucucuguuas(invAb) | 360 |
| AD07713 | usGfsusAfaAfaAfgGfcAfaCfaGfaGfaGfsg | 299 | (NAG37)s(invAb)sccucucugUfUfGfccuuuuuacas(invAb) | 361 |
| AD07714 | usUfsusAfcAfaAfaGfcAfaAfaCfaGfgUfsc | 300 | (NAG37)s(invAb)sgaccuguuUfUfGfcuuuuguaa_2Nas | 362 |
| | | | (invAb) | |
| AD07802 | usUfsuscaaguuacAfaAfaGfcaaasc | 301 | (NAG37)s(invAb)sguuugcUfuUfuGfuaacuugaaas(invAb) | 363 |
| AD07911 | asAfsgsUfuAfcAfaAfaGfcAfaAfaCfaGfsc | 302 | (NAG37)s(invAb)sgcuguuuuGfCfUfuuuguaacuus(invAb) | 364 |
| AD07912 | asAfsgsUfuAfcAfaAfaGfcAfaAfaCfaGfsg | 292 | (NAG37)s(invAb)sccuguuuuGfCfUfuuugua_2Nacuus | 365 |
| | | | (invAb) | |
| AD07913 | cPrpasAfsgsUfuAfcAfaAfaGfcAfaAfaCfa | 303 | (NAG37)s(invAb)sccuguuuuGfCfUfuuuguaacuus(invAb) | 354 |
| | Gfsg | | | |
| AD07914 | asAfsgUfuAfcAfaAfaGfcAfaAfaCfasGfsg | 304 | (NAG37)s(invAb)sccuguuuuGfCfUfuuuguaacuus(invAb) | 354 |
| AD07915 | asAfsgsUfuAfcAfaAfaGfcAfaAfaCfaGfsg | 292 | (NAG37)s(invAb)sccuguuUfuGfcUfuuuguaacuus(invAb) | 366 |
| AD07916 | asAfsgsUfuAfcAfaAfaGfcAfaAfaCfaGfsg | 292 | (NAG37)s(invAb)sccuguuuuGfcUfUfuuguaacuus(invAb) | 367 |
| AD07917 | asAfsgsUfuAfcAfaAfaGfcAfaAfaCfaGfsg | 292 | (NAG37)s(invAb)sccuguuuuGfcUfuUfuguaacuus(invAb) | 368 |
| AD07918 | asAfsgsuuacaaaaGfcAfaAfacagsg | 305 | (NAG37)s(invAb)sccuguuuuGfCfUfuuuguaacuus(invAb) | 354 |
| AD07919 | asAfsGfsuuacaaaaGfcAfaAfacagsg | 306 | (NAG37)s(invAb)sccuguuuuGfCfUfuuuguaacuus(invAb) | 354 |
| AD08105 | asAfsgsUfuAfcAfaAfaGfcAfaAfaCfaGfsg | 292 | (NAG37)s(invAb)sccuguuuuGfcUfuUfugua_2Nacuus | 369 |
| | | | (invAb) | |
| AD08106 | asAfsgsuuacaaaaGfcAfaAfacagsg | 305 | (NAG37)s(invAb)sccuguuuuGfCfUfuuugua_2Nacuus | 365 |
| | | | (invAb) | |
| AD08107 | asAfsgsuuacaaaaGfcAfaAfacagsg | 305 | (NAG37)s(invAb)sccuguuuuGfcUfuUfugua_2Nacuus | 369 |
| | | | (invAb) | |
| AD08108 | cPrpasAfsgsuuacaaaaGfcAfaAfacagsg | 307 | (NAG37)s(invAb)sccuguuuuGfcUfuUfugua_2Nacuus | 369 |
| | | | (invAb) | |
| AD08109 | asAfsguuacaaaaGfcAfaAfacasgsg | 308 | (NAG37)s(invAb)sccuguuuuGfcUfuUfugua_2Nacuus | 369 |
| | | | (invAb) | |
| AD08110 | cPrpasAfsguuacaaaaGfcAfaAfacasgsg | 309 | (NAG37)s(invAb)sccuguuuuGfcUfuUfugua_2Nacuus | 369 |
| | | | (invAb) | |
| AD08574 | asAfsgsuuacaaaaGfcAfaAfacasgsg | 310 | (NAG37)s(invAb)sccuguuuuGfCfUfuuugua_2Nacuus | 365 |
| | | | (invAb) | |
| AD08575 | asAfsgsuuacaaaaGfcAfaAfacasgsg | 310 | (NAG37)s(invAb)sccuguuuuGfcUfUfuugua_2Nacuus | 370 |
| | | | (invAb) | |
| AD08576 | asAfsgsuuacaaaaGfcAfaAfacasgsg | 310 | (NAG37)s(invAb)sccuguuuuGfcUfuuugua_2Nacuus | 371 |
| | | | (invAb) | |
| AD08577 | usAfsgsuuacaaaaGfcAfaAfacasgsg | 311 | (NAG37)s(invAb)sccuguuuuGfCfUfuuugua_2Nacuas | 372 |
| | | | (invAb) | |
| AD08578 | cPrpusAfsgsuuacaaaaGfcAfaAfacasgsg | 312 | (NAG37)s(invAb)sccuguuuuGfCfUfuuugua_2Nacuas | 372 |
| | | | (invAb) | |
| AD08579 | cPrpusAfsguuacaaaaGfcAfaAfacasgsg | 313 | (NAG37)s(invAb)sccuguuuuGfCfUfuuugua_2Nacuas | 372 |
| | | | (invAb) | |
| AD08580 | cPrpusAfguuacaaaaGfcAfaAfacasgsg | 314 | (NAG37)s(invAb)sccuguuuuGfCfUfuuugua_2Nacuas | 372 |
| | | | (invAb) | |
| AD08581 | cPrpuAfguuacaaaaGfcAfaAfacasgsg | 315 | (NAG37)s(invAb)sccuguuuuGfCfUfuuugua_2Nacuas | 372 |
| | | | (invAb) | |
| AD08631 | cPrpusUfsguaa_2NaaaggCfaAfcAfgagsas | 316 | (NAG37)s(invAb)scucucuguUfgCfcUfuuuuacaas(invAb) | 373 |
| | g | | | |
| AD08796 | asGfsucgcU_{UNA}ggagGfcAfcCfaausgsg | 317 | (NAG37)s(invAb)sccauugguGfcCfuCfcagcgacus(invAb) | 374 |
| AD08834 | cPrpusCfcaaaagauaAfaUfgUfcugcsu | 318 | (NAG37)s(invAb)sagcagacaUfUfUfaucuuuuigas(invAb) | 375 |
| AD08835 | asGfsagaggacagAfcCfcAfaaasgsg | 319 | (NAG37)s(invAb)sccuuuuggGfUfCfuguccucucus(invAb) | 376 |
| AD08836 | asAfsasuaucuucaAfgUfuAfcaaasg | 320 | (NAG37)s(invAb)scuuuguaaCfUfUfgaagauauuus(invAb) | 377 |
| AD08837 | cPrpusAfsasauaucuucAfaGfuUfacaasg | 321 | (NAG37)s(invAb)scuuguaacUfUfGfaagauauuuas(invAb) | 378 |
| AD08838 | cPrpusGfuagcA_{UNA}ggcaGfcAfcCfuggcsa | 322 | (NAG37)s(invAb)sugccagguGfCfUfgccugcuacas(invAb) | 379 |
| AD08839 | asGfsgacagacccAfaAfaGfauasasg | 323 | (NAG37)s(invAb)scuuaucuuUfUfGfggucuiuccus(invAb) | 380 |
| AD08840 | cPrpusAfsasaugucugcUfuGfcUfugggsu | 324 | (NAG37)s(invAb)sacccaagcAfAfGfcagacauuuas(invAb) | 381 |
| AD08841 | cPrpusCfsgaagU_{UNA}cgguGfaCfcAfugacsc | 325 | (NAG37)s(invAb)sggucauggUfCfAfccgacuucgas(invAb) | 382 |
| AD08842 | asAfsasacaggucuAfgAfaAfaguusg | 326 | (NAG37)s(invAb)scaacuuuuCfUfAfgaccuguuuus(invAb) | 383 |
| AD08843 | cPrpusGfsaugacaucuUfuGfgCfagasgsg | 327 | (NAG37)s(invAb)sccucugccAfAfAfgaugucaucas(invAb) | 384 |
| AD08844 | cPrpusUfsusuccgaauaAfaCfuCfcaggsc | 328 | (NAG37)s(invAb)sgccuggagUfUfUfauucggaaa_2Nas | 385 |
| | | | (invAb) | |
| AD08845 | asUfsasaauaucuuCfaAfgUfuacasg | 329 | (NAG37)s(invAb)scuguaacuUfGfAfagauauuua_2Nus | 386 |
| | | | (invAb) | |
| AD08878 | cPrpuAfguuacaaaaGfcAfaAfacasgsg | 315 | (NAG37)s(invAb)sccuguuuuGfcUfUfuugua_2Nacuas | 387 |
| | | | (invAb) | |
| AD08879 | cPrpuAfguuacaaaaGfcAfaAfacasgsg | 315 | (NAG37)s(invAb)sccuguuuuGfcUfUfuuguaacuas(invAb) | 388 |
| AD08880 | cPrpuAfguuacaaaaGfcAfaAfacasgsg | 315 | (NAG37)s(invAb)sccuguuuuGfCfUfuuuguaacuas(invAb) | 389 |
| AD09027 | cPrpusUfsguaa_2NaaaggCfaAfcAfgagsas | 316 | (NAG37)s(invAb)scucucuguUfGfCfcuuuuuacaas(invAb) | 350 |
| | g | | | |
| AD09028 | cPrpusUfsguaaaaaggCfaAfcAfgagsasg | 330 | (NAG37)s(invAb)scucucuguUfGfCfcuuuuuacaas(invAb) | 350 |
| AD09029 | cPrpuUfguaaaaaggCfaAfcAfgagsasg | 331 | (NAG37)s(invAb)scucucuguUfGfCfcuuuuuacaas(invAb) | 350 |
| AD09030 | cPrpuUfguaa_2NaaaggCfaAfcAfgagsasg | 332 | (NAG37)s(invAb)scucucuguUfGfCfcuuuuuacaas(invAb) | 350 |
| AD09031 | cPrpuUfguaa_2NaaaggCfaAfcAfgagsasg | 332 | (NAG37)s(invAb)scucucuguUfgCfCfuuuuuacaas(invAb) | 390 |
| AD09032 | cPrpuUfguaaaaaggCfaAfcAfgagsasg | 331 | (NAG37)s(invAb)scucucuguUfgCfCfuuuuuacaas(invAb) | 390 |
| AD09033 | cPrpuUfguaaaaaggCfaAfcAfgagasg | 333 | (NAG37)s(invAb)scucucuguUfgCfCfuuuuuacaas(invAb) | 390 |
| AD09034 | cPrpuUfguaaaaaggCfaAfcAfgagasg | 333 | (NAG37)s(invAb)scucucuguuGfCfcuuuuuacaas(invAb) | 391 |
| AD09038 | cPrpuAfaauaucuucAfaGfuUfacaasg | 334 | (NAG37)s(invAb)scuuguaacUfUfGfaagauauuuas(invAb) | 378 |
| AD09039 | cPrpuAfaauaucuucAfaGfuUfacagsg | 335 | (NAG37)s(invAb)sccuguaacUfUfGfaagauauuuas(invAb) | 392 |
| AD09040 | cPrpusAfaauaucuucAfaGfuUfacagsg | 336 | (NAG37)s(invAb)sccuguaacUfUfGfaagauauuuas(invAb) | 392 |
| AD09041 | cPrpusAfaauaucuucAfaGfuUfacagsg | 336 | (NAG37)s(invAb)sccuguaacUfUfGfaagaua_2Nuuuas | 393 |
| | | | (invAb) | |
| AD09042 | cPrpaGfagaggacagAfcCfcAfaaagsg | 337 | (NAG37)s(invAb)sccuuuuggGfUfCfuguccucucus(invAb) | 376 |
| AD09043 | cPrpuGfagaggacagAfcCfcAfaaagsg | 338 | (NAG37)s(invAb)sccuuuuggGfUfCfuguccucucas(invAb) | 394 |
| AD09059 | cPrpaAfaacaggucuAfgAfaAfaguusg | 339 | (NAG37)s(invAb)scaacuuuuCfUfAfgaccuguuuus(invAb) | 383 |
| AD09131 | cPrpasAfsgsuuacaaaaGfcAfaAfacagsg | 307 | (NAG37)s(invAb)sccuguuuuGfCfUfuuuguaacuus(invAb) | 354 |
| AD09132 | cPrpaAfguuacaaaaGfcAfaAfacagsg | 340 | (NAG37)s(invAb)sccuguuuuGfCfUfuuuguaacuus(invAb) | 354 |
| AD09327 | cPrpasAfsguuacaaaaGfcAfaAfacasgsg | 309 | (NAG37)s(invAb)sccuguuuuGfcUfuUfuguaacuus(invAb) | 368 |
| AD09851 | cPrpasAfsgUfuacaaaaGfcAfaAfacasgsg | 342 | (NAG37)s(invAb)sccuguuuuGfcUfuUfuguaacuus(invAb) | 368 |
| AD09852 | cPrpasAfsguuAfcaaaaGfcAfaAfacasgsg | 343 | (NAG37)s(invAb)sccuguuuuGfcUfuUfuguaacuus(invAb) | 368 |
| AD09853 | cPrpasAfsguuacAfaaaGfcAfaAfacasgsg | 344 | (NAG37)s(invAb)sccuguuuuGfcUfuUfuguaacuus(invAb) | 368 |
| AD09854 | cPrpasAfsguuacaaAfaGfcAfaAfacasgsg | 345 | (NAG37)s(invAb)sccuguuuuGfcUfuUfuguaacuus(invAb) | 368 |
| AD09855 | cPrpasAfsguuacaaaaGfcAfaAfaCfasgsg | 346 | (NAG37)s(invAb)sccuguuuuGfcUfuUfuguaacuus(invAb) | 368 |

In some embodiments, a PCSK9 RNAi agent is prepared or provided as a salt, mixed salt, or a free-acid. The RNAi agents described herein, upon delivery to a cell expressing a PCSK9 gene, inhibit or knockdowN expression of one or more PCSK9 genes in vivo and/or in vitro.

### Targeting Ligands or Groups, Linking Groups, and Delivery Vehicles

In some embodiments, a PCSK9 RNAi agent is conjugated to one or more non-nucleotide groups including, but not limited to, a targeting group, a linking group, a targeting ligand, a delivery polymer, or a delivery vehicle. The non-nucleotide group can enhance targeting, delivery or attachment of the RNAi agent. Examples of targeting groups and linking groups are provided in Table 6. The non-nucleotide group can be covalently linked to the 3′ and/or 5′ end of either the sense strand and/or the antisense strand. In some embodiments, a PCSK9 RNAi agent contains a non-nucleotide group linked to the 3′ and/or 5′ end of the sense strand. In some embodiments, a non-nucleotide group is linked to the 5′ end of a PCSK9 RNAi agent sense strand. A non-nucleotide group may be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the RNAi agent or conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules.

In some embodiments, a targeting group is linked to an RNAi agent using a linker, such as a PEG linker or one, two, or three abasic and/or ribitol (abasic ribose) residues, which can in some instances serve as linkers. In some embodiments, a targeting ligand comprises a galactose-derivative cluster.

The PCSK9 RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

In some embodiments, a targeting group comprises an asialoglycoprotein receptor ligand. As used herein, an asialoglycoprotein receptor ligand is a ligand that contains a moiety having affinity for the asialoglycoprotein receptor. As noted herein, the asialoglycoprotein receptor is highly expressed on hepatocytes. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formyl-galactosamine, N-acetyl-galactosamine, N-propionyl-galactosamine, N-n-butanoyl-galactosamine, and N-iso-butanoylgalactos-amine (see for example: S. T. Iobst and K. Drickamer, J. B. C., 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative, also referred to as monovalent or monodentate) or multimeric (e.g., having multiple galactose derivatives). The galactose derivative or galactose derivative cluster can be attached to the 3′ or 5′ end of the sense or antisense strand of the RNAi agent using methods known in the art.

The preparation of targeting ligands, such as galactose derivative clusters, is described in, for example, International Patent Application Publication No. WO 2018/044350 to Arrowhead Pharmaceuticals, Inc., and International Patent Application Publication No. WO 2017/156012 to Arrowhead Pharmaceuticals, Inc., the contents of both of which are incorporated by reference herein in their entirety.

As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamine moieties. In some embodiments, the galactose derivative cluster comprises three N-acetyl-galactosamine moieties. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamine moieties.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, e.g., U.S. Pat. No. 5,885,968; Biessen et al. J. Med. Chem. 1995 Vol. 39 p. 1538-1546). In some embodiments, the PEG spacer is a PEG3 spacer. The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

Embodiments of the present disclosure include pharmaceutical compositions for delivering a PCSK9 RNAi agent to a liver cell in vivo. Such pharmaceutical compositions can include, for example, a PCSK9 RNAi agent conjugated to a galactose derivative cluster. In some embodiments, the galactose derivative cluster is comprised of a galactose derivative trimer, which can be, for example, an N-acetyl-galactosamine trimer, or galactose derivative tetramer, which can be, for example, an N-acetyl-galactosamine tetramer.

A targeting ligand or targeting group can be linked to the 3′ or 5′ end of a sense strand or an antisense strand of a PCSK9 RNAi agent disclosed herein.

Targeting ligands include, but are not limited to (NAG37) and (NAG37)s as defined in Table 6. Other targeting groups and targeting ligands, including galactose cluster targeting ligands, are known in the art.

In some embodiments, a linking group is conjugated to the RNAi agent. The linking group facilitates covalent linkage of the agent to a targeting group, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, can include, but are not limited to: reactive groups such a primary amines and alkynes, alkyl groups, abasic nucleotides, ribitol (abasic ribose), and/or PEG groups.

In some embodiments, a targeting group is linked internally to a nucleotide on the sense strand and/or the antisense strand of the RNAi agent. In some embodiments, a targeting group is linked to the RNAi agent via a linker.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage can optionally include a spacer that increases the distance between the two joined atoms. A spacer can further add flexibility and/or length to the linkage. Spacers include, but are not limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, when two or more RNAi agents are included in a single composition, each of the RNAi agents may be linked to the same targeting group or two a different targeting groups (i.e., targeting groups having different chemical structure). In some embodiments, targeting groups are linked to the PCSK9 RNAi agents disclosed herein without the use of an additional linker. In some embodiments, the targeting group itself is designed having a linker or other site to facilitate conjugation readily present. In some embodiments, when two or more PCSK9 RNAi agents are included in a single molecule, each of the RNAi agents may utilize the same linker or different linkers (i.e., linkers having different chemical structures).

Any of the PCSK9 RNAi agent nucleotide sequences listed in Tables 2, 3, 4, 5C, 7A, 7B, or 8 whether modified or unmodified, can contain 3′ and/or 5′ targeting group(s) or linking group(s). Any of the PCSK9 RNAi agent sequences listed in Table 3 or 4, or are otherwise described herein, which contain a 3′ or 5′ targeting group or linking group, can alternatively contain no 3′ or 5′ targeting group or linking group, or can contain a different 3′ or 5′ targeting group or linking group including, but not limited to, those depicted in Table 6. Any of the PCSK9 RNAi agent duplexes listed in Tables 5A, 5B, 5C, and 8, whether modified or unmodified, can further comprise a targeting group or linking group, including, but not limited to, those depicted in Table 6, and the targeting group or linking group can be attached to the 3′ or 5′ terminus of either the sense strand or the antisense strand of the PCSK9 RNAi agent duplex.

Examples of targeting groups and linking groups (which when combined can form targeting ligands) are provided in Table 6. Table 4, Table 5C, and Table 8 provide certain embodiments of PCSK9 RNAi agent sense strands having a targeting group or linking group linked to the 5′ or 3′ end.

TABLE 6
Structures Representing Various Modified Nucleotides, Targeting Ligands or
Targeting Groups, Capping Residues, and Linkage Groups

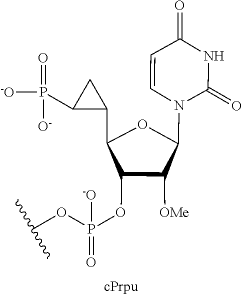

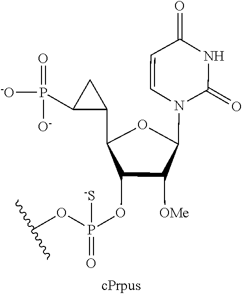

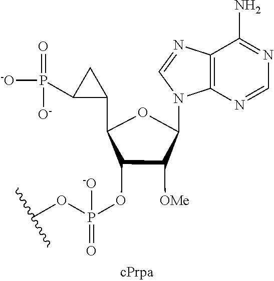

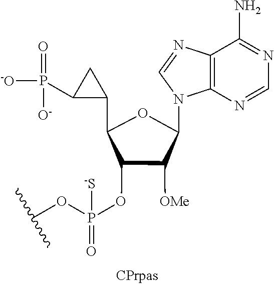

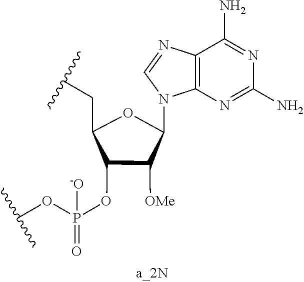

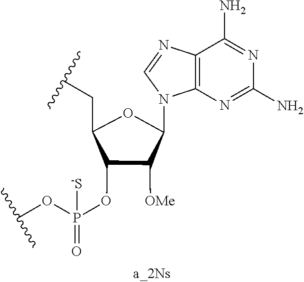

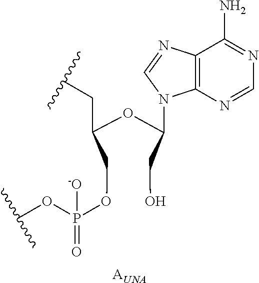

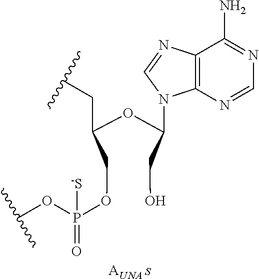

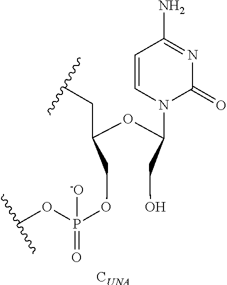

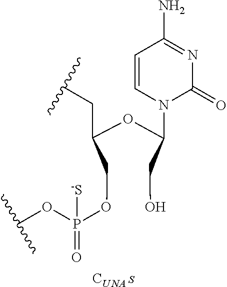

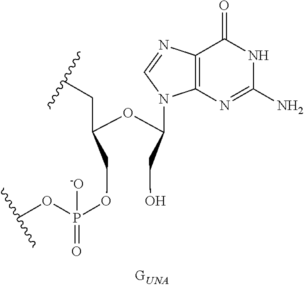

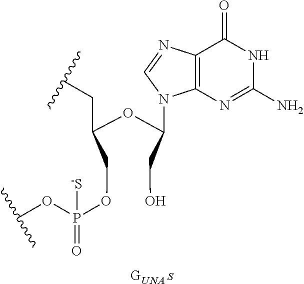

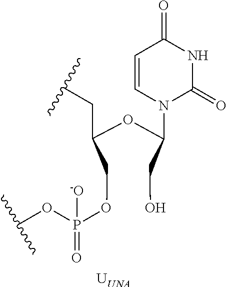

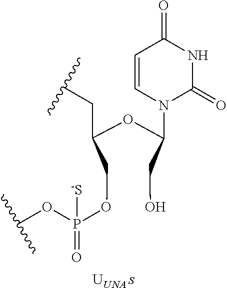

When positioned internally:

| | |
|---|---|
| 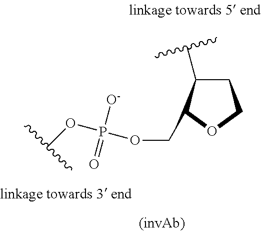 | 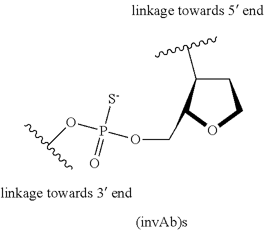 |

When positioned at the 3′ terminal end:

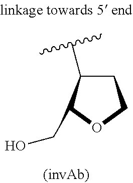

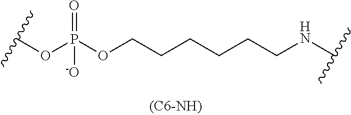

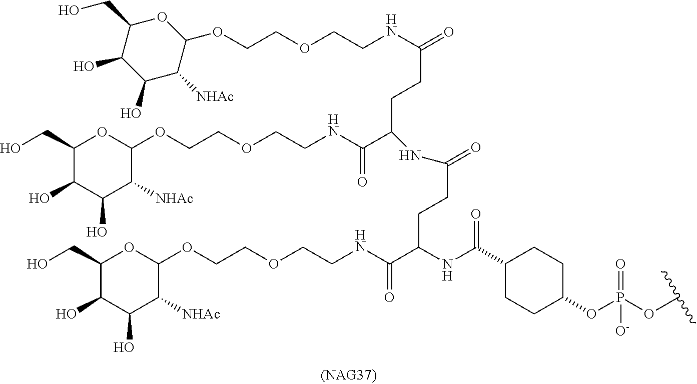 [SMILES: *OP(=O)([O-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

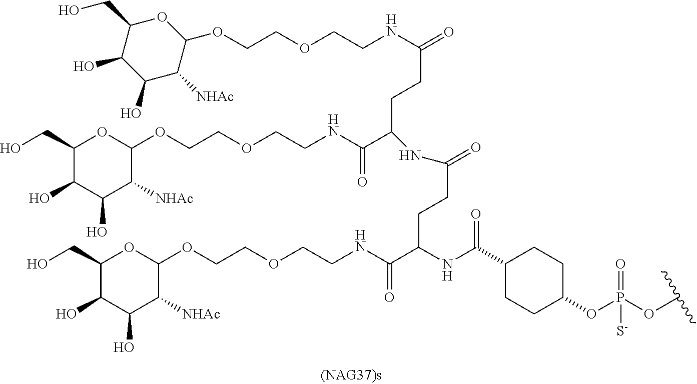 [SMILES: *OP(=O)([S-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

In each of the above structures in Table 6, NAG comprises an N-acetyl-galactosamine. In some embodiments, NAG as depicted in Table 6 above can comprise another galactose derivative that has affinity for the asialoglycoprotein receptor present on hepatocytes, as would be understood by a person of ordinary skill in the art to be attached in view of the structures above and description provided herein. Other linking groups known in the art may be used.

In some embodiments, a delivery vehicle can be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine. In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/0022309, WO 2011/104169, and WO 2012/083185, WO 2013/032829, WO 2013/158141, each of which is incorporated herein by reference), hydrogels, cyclodextrins, biodegradable nanocapsules, and bioadhesive microspheres, proteinaceous vectors, or other delivery systems suitable for nucleic acid or oligonucleotide delivery as known and available in the art.

### Pharmaceutical Compositions

The PCSK9 RNAi agents disclosed herein can be prepared as pharmaceutical compositions or formulations (also referred to herein as “medicaments”). In some embodiments, pharmaceutical compositions include at least one PCSK9 RNAi agent. These pharmaceutical compositions are particularly useful in the inhibition of the expression of the target mRNA in a target cell, a group of cells, a tissue, or an organism (such as a human).

The pharmaceutical compositions can be used to treat a subject or patient having a disease, disorder, or condition that would benefit from reduction in the level of PCSK9 mRNA and/or the PCSK9 protein translated therefrom. The pharmaceutical compositions can be used to treat a subject at risk of developing a disease, disorder, symptom, or condition that would benefit from reduction of the level of the target mRNA or an inhibition in expression the target gene. In one embodiment, the method includes administering a PCSK9 RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a PCSK9 RNAi agent, thereby forming a pharmaceutical formulation or medicament suitable for in vivo delivery to a subject, including a human.

The pharmaceutical compositions that include a PCSK9 RNAi agent and methods disclosed herein decrease the level of the target mRNA in a cell, group of cells, tissue, organ, or subject, including by administering to the subject a therapeutically effective amount of a herein described PCSK9 RNAi agent, thereby inhibiting the expression or translation of PCSK9 mRNA in the subject. In some embodiments, the subject has been previously identified as having a pathogenic upregulation of the target gene in hepatocytes. In some embodiments, the subject has been previously identified or diagnosed as having hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease. In some embodiments, the subject has been suffering from symptoms associated with hypercholesterolemia. In some embodiments, the subject would benefit from a reduction of PCSK9 gene expression in the subject's liver.

In some embodiments, the described pharmaceutical compositions including a PCSK9 RNAi agent are used for treating or managing clinical presentations associated with including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease. Other diseases or conditions for which a PCSK9 RNAi agent may be useful include coronary artery disease, polygenic dyslipidemia, or heart disease. In some embodiments, a therapeutically (including prophylactically) effective amount of one or more of pharmaceutical compositions is administered to a subject in need of such treatment. In some embodiments, administration of any of the disclosed PCSK9 RNAi agents can be used to decrease the number, severity, and/or frequency of symptoms of a disease in a subject.

The described pharmaceutical compositions that include a PCSK9 RNAi agent can be used to treat at least one symptom in a subject having a disease or disorder that would benefit from reduction or inhibition in expression of PCSK9 mRNA and/or a reduction in PCSK9 protein levels. Measuring PCSK9 levels can be conducted in accordance with established methods known in the art. The Examples disclosed herein provide

In some embodiments, the subject is administered a therapeutically effective amount of one or more pharmaceutical compositions that include a PCSK9 RNAi agent thereby treating the symptom. In other embodiments, the subject is administered a prophylactically effective amount of one or more PCSK9 RNAi agents, thereby preventing or inhibiting the at least one symptom.

The route of administration is the path by which a PCSK9 RNAi agent is brought into contact with the body. In general, methods of administering drugs and oligonucleotides and nucleic acids for treatment of a mammal are well known in the art and can be applied to administration of the compositions described herein. The PCSK9 RNAi agents disclosed herein can be administered via any suitable route in a preparation appropriately tailored to the particular route. Thus, herein described pharmaceutical compositions can be administered by injection, for example, intravenously, intramuscularly, intracutaneously, subcutaneously, intraarticularly, or intraperitoneally. In some embodiments, the herein described pharmaceutical compositions are administered via subcutaneous injection.

The pharmaceutical compositions including a PCSK9 RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., PCSK9 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® ELTM (BASF, Parsippany, NJ) or phosphate buffered saline (PBS). Suitable carriers should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

In some embodiments, pharmaceutical formulations that include the PCSK9 RNAi agents disclosed herein suitable for subcutaneous administration can be prepared in an aqueous sodium phosphate buffer (e.g., the PCSK9 RNAi agent formulated in 0.5 mM sodium phosphate monobasic, 0.5 mM sodium phosphate dibasic, in water). In some embodiments, pharmaceutical formulations that include the PCSK9 RNAi agents disclosed herein suitable for subcutaneous administration can be prepared in water for injection (sterile water). PCSK9 RNAi agents disclosed herein suitable for subcutaneous administration can be prepared in isotonic saline (0.9%).

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for oral administration of the PCSK9 RNAi agents disclosed herein can also be prepared. In some embodiments, the PCSK9 RNAi agents disclosed herein are administered orally. In some embodiments, the PCSK9 RNAi agents disclosed herein are formulated in a capsule for oral administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The PCSK9 RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, analgesics, antihistamines, or anti-inflammatory agents (e.g., acetaminophen, NSAIDs, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another PCSK9 RNAi agent (e.g., a PCSK9 RNAi agent that targets a different sequence within the PCSK9 target). In other embodiments, the second therapeutic can be a small molecule drug, an antibody, an antibody fragment, or an aptamer.

In some embodiments, the described PCSK9 RNAi agent(s) are optionally combined with one or more additional therapeutics. The PCSK9 RNAi agent and additional therapeutic(s) can be administered in a single composition or they can be administered separately. In some embodiments, the one or more additional therapeutics is administered separately in separate dosage forms from the RNAi agent (e.g., the PCSK9 RNAi agent is administered by subcutaneous injection, while the additional therapeutic involved in the method of treatment dosing regimen is administered orally). In some embodiments, the described PCSK9 RNAi agent(s) are administered to a subject in need thereof via subcutaneous injection, and the one or more optional additional therapeutics are administered orally, which together provide for a treatment regimen for diseases and conditions associated with including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease. In some embodiments, the described PCSK9 RNAi agent(s) are administered to a subject in need thereof via subcutaneous injection, and the one or more optional additional therapeutics are administered via a separate subcutaneous injection. In some embodiments, the PCSK9 RNAi agent and one or more additional therapeutics are combined into a single dosage form (e.g., a “cocktail” formulated into a single composition for subcutaneous injection). The PCSK9 RNAi agents, with or without the one or more additional therapeutics, can be combined with one or more excipients to form pharmaceutical compositions.

Generally, an effective amount of a PCSK9 RNAi agent will be in the range of from about 0.1 to about 100 mg/kg of body weight/dose, e.g., from about 1.0 to about 50 mg/kg of body weight/dose. In some embodiments, an effective amount of an active compound will be in the range of from about 0.25 to about 5 mg/kg of body weight per dose. In some embodiments, an effective amount of an active ingredient will be in the range of from about 0.5 to about 4 mg/kg of body weight per dose. In some embodiments, an effective amount of a PCSK9 RNAi agent may be a fixed dose. In some embodiments, the fixed dose is in the range of from about 5 mg to about 1,000 mg of PCSK9 RNAi agent. In some embodiments, the fixed does is in the range of 50 to 400 mg of PCSK9 RNAi agent. Dosing may be weekly, bi-weekly, monthly, quarterly, or at any other interval depending on the dose of PCSK9 RNAi agent administered, the activity level of the particular PCSK9 RNAi agent, and the desired level of inhibition for the particular subject. The Examples herein show suitable levels for inhibition in certain animal species. The amount administered will depend on such variables as the overall health status of the patient or subject, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum.

For treatment of disease or for formation of a medicament or composition for treatment of a disease, the pharmaceutical compositions described herein including a PCSK9 RNAi agent can be combined with an excipient or with a second therapeutic agent or treatment including, but not limited to: a second or other RNAi agent, a small molecule drug, an antibody, an antibody fragment, peptide and/or an aptamer.

The described PCSK9 RNAi agents, when added to pharmaceutically acceptable excipients or adjuvants, can be packaged into kits, containers, packs, or dispensers. The pharmaceutical compositions described herein may be packaged in pre-filled syringes, pen injectors, autoinjectors, infusion bags/devices, or vials.

### PCSK9 RNAi Agent Drug Substances and Formulations

In some embodiments, the PCSK9 RNAi agent disclosed herein has the nucleotide sequences of the PCSK9 RNAi Drug Substance shown in Table 8, below. The nucleotide sequences of the PCSK9 RNAi agent found in PCSK9 RNAi Drug Substance include an antisense strand nucleotide sequence as set forth in the following Table 7A, and a sense strand nucleotide sequence as set forth in the following Table 7B.

TABLE 7A
PCSK9 RNAi Agent Antisense Strand Sequence

| SEQ | Antisense | SEQ | Underlying |
|---|---|---|---|
| ID | Sequence (Modified) | ID | Base Sequence |
| NO. | (5′ → 3′) | NO. | (5′ → 3′) |
| 309 | cPrpasAfsguuacaaaaGfcAfaAfacasgsg | 401 | AAGUUACAAAAGCAAAACAGG |

TABLE 7B
PCSK9 RNAi Agent Sense Strand Nucleotide Sequence
(shown as modified version without inverted abasic
residues or NAG targeting group present in PCSK9
RNAi Drug Substance)

| SEQ | | SEQ | Underlying |
|---|---|---|---|
| ID | Sense Sequence (Modified) | ID | Base Sequence |
| NO. | (5′ → 3′) | NO. | (5′ → 3′) |
| 368 | (NAG37)s(invAb)sccuguuuuG | 430 | CCUGUUUUGCUUU |
| | fcUfuUfuguaacuus(invAb) | | UGUAACUU |

As used in Tables 7A, 7B, and 8 herein, the following notations are used to indicate modified nucleotides, targeting groups, and linking groups: A, C, G, and U represent adenosine, cytidine, guanosine, and uridine, respectively; a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue (see Table 6); and (NAG37)s represents the following structure (depicted as both a sodium salt and free acid):

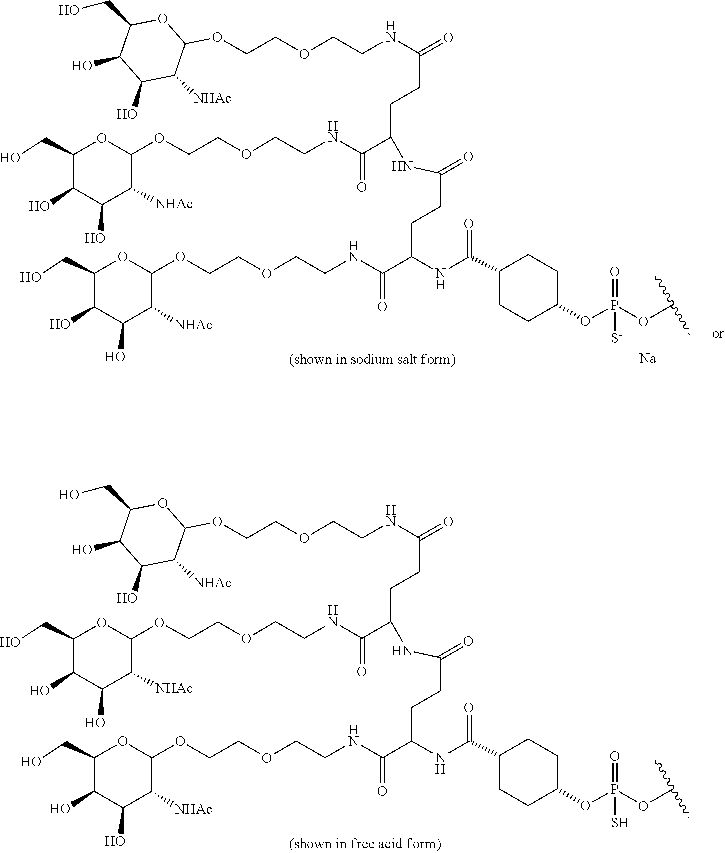

Each sense strand and/or antisense strand can have any targeting groups or linking groups listed above, as well as other targeting or linking groups, conjugated to the 5′ and/or 3′ end of the sequence.

The PCSK9 RNAi agent antisense strand sequence is designed to target mRNA transcripts from a PCSK9 gene in a human subject, thereby silencing translation of PCSK9 protein using an RNA interference mechanism for human subjects with PCSK9.

In some embodiments, the methods disclosed herein use the PCSK9 RNAi Drug Substance set forth in the following Table 8:

TABLE 8
PCSK9 RNAi Drug Substance
Sense and Antisense Strands (The sense and antisense strands are annealed to form a duplex):

| | | |
|---|---|---|
| Sense Strand (Modified Sequence) (5′ → 3′): | (NAG37)s(invAb)sccuguuuuGfcUfuUfuguaacuus(invAb) | (SEQ ID NO: 368) |
| Antisense Strand (Modified Sequence) (5′ → 3′): | cPrpasAfsguuacaaaaGfcAfaAfacasgsg | (SEQ ID NO: 309) |

In some embodiments, the PCSK9 RNAi Drug Substance is prepared or provided as a salt, mixed salt, or a free acid. In some embodiments, the form is a sodium salt.

In some embodiments, the PCSK9 RNAi Drug Substance as provided in Table 8 is formulated with one or more pharmaceutically acceptable excipients to form a pharmaceutical composition suitable for administration to a human subject. In some embodiments, the PCSK9 RNAi Drug Substance described in Table 8 is formulated at 200 mg/mL in an aqueous sodium phosphate buffer (0.5 mM sodium phosphate monobasic, 0.5 mM sodium phosphate dibasic), which is suitable for subcutaneous administration in humans.

### Methods of Treatment and Inhibition of Expression

The PCSK9 RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from reduction and/or inhibition in expression of PCSK9 mRNA and/or PCSK9 protein levels, for example, a subject that has been diagnosed with or is suffering from symptoms related to including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease.

In some embodiments, the subject is administered a therapeutically effective amount of any one or more PCSK9 RNAi agents. Treatment of a subject can include therapeutic and/or prophylactic treatment. The subject is administered a therapeutically effective amount of any one or more PCSK9 RNAi agents described herein. The subject may be an adult, adolescent, child, or infant. Administration of a pharmaceutical composition described herein can be to a human being or animal.

The PCSK9 RNAi agents described herein can be used to treat at least one symptom in a subject having a PCSK9-related disease or disorder, or having a disease or disorder that is mediated at least in part by PCSK9 gene expression. In some embodiments, the PCSK9 RNAi agents are used to treat or manage a clinical presentation of a subject with a disease or disorder that would benefit from or be mediated at least in part by a reduction in PCSK9 mRNA or PCSK9 protein levels. The subject is administered a therapeutically effective amount of one or more of the PCSK9 RNAi agents or PCSK9 RNAi agent-containing compositions described herein. In some embodiments, the methods disclosed herein comprise administering a composition comprising a PCSK9 RNAi agent described herein to a subject to be treated. In some embodiments, the subject is administered a prophylactically effective amount of any one or more of the described PCSK9 RNAi agents, thereby treating the subject by preventing or inhibiting the at least one symptom.

In certain embodiments, the present disclosure provides methods for treatment of diseases, disorders, conditions, or pathological states mediated at least in part by PCSK9 gene expression, in a patient in need thereof, wherein the methods include administering to the patient any of the PCSK9 RNAi agents described herein.

In some embodiments, the gene expression level and/or mRNA level of a PCSK9 gene in a subject to whom a described PCSK9 RNAi agent is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the PCSK9 RNAi agent or to a subject not receiving the PCSK9 RNAi agent. The PCSK9 mRNA level in the subject may be reduced in a cell, group of cells, and/or tissue of the subject. In some embodiments, the PCSK9 gene expression is inhibited by at least about 30%, 35%, 40%, 45% 50%, 55%, 60%, 65%, or greater than 65% in hepatocytes relative to the subject prior to being administered the PCSK9 RNAi agent or to a subject not receiving the PCSK9 RNAi agent.

In some embodiments, the PCSK9 protein level in a subject to whom a described PCSK9 RNAi agent has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the PCSK9 RNAi agent or to a subject not receiving the PCSK9 RNAi agent. The protein level in the subject may be reduced in a cell, group of cells, tissue, blood, and/or other fluid of the subject.

A reduction in PCSK9 mRNA levels and PCSK9 protein levels can be assessed by any methods known in the art. As used herein, a reduction or decrease in PCSK9 mRNA level and/or protein level are collectively referred to herein as a reduction or decrease in PCSK9 or inhibiting or reducing the gene expression of PCSK9. The Examples set forth herein illustrate known methods for assessing inhibition of PCSK9 gene expression. The person of ordinary skill in the art would further know suitable methods for assessing inhibition of PCSK9 gene expression in vivo and/or in vitro.

In some embodiments, disclosed herein are methods of treatment (including prophylactic or preventative treatment) of diseases, disorders, or symptoms caused by caused by including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease, wherein the methods include administering to a subject in need thereof a therapeutically effective amount of a PCSK9 RNAi agent that includes an antisense strand that is at least partially complementary to the portion of the PCSK9 mRNA having the sequence in Table 1. In some embodiments, disclosed herein are methods of treatment (including prophylactic or preventative treatment) of diseases or symptoms caused by caused by including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease, wherein the methods include administering to a subject in need thereof a therapeutically effective amount of a PCSK9 RNAi agent that includes an antisense strand comprising the sequence of any of the sequences in Tables 2, 3, 5C, 7A, or 8, and a sense strand that comprises any of the sequences in Tables 2, 4, 5C, 7B, or 8 that is at least partially complementary to the antisense strand. In some embodiments, disclosed herein are methods of treatment (including prophylactic or preventative treatment) of diseases or symptoms caused by caused by including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease, wherein the methods include administering to a subject in need thereof a therapeutically effective amount of a PCSK9 RNAi agent that includes a sense strand that comprises any of the sequences in Tables 2, 4, 5C, 7B, or 8 and an antisense strand comprising the sequence of any of the sequences in Tables 2, 3, 5C, 7A, or 8 that is at least partially complementary to the sense strand.

In some embodiments, disclosed herein are methods for inhibiting expression of a PCSK9 gene in a cell, wherein the methods include administering to the cell a PCSK9 RNAi agent that includes an antisense strand that is at least partially complementary to the portion of the PCSK9 mRNA having the sequence in Table 1. In some embodiments, disclosed herein are methods of inhibiting expression of a PCSK9 gene in a cell, wherein the methods include administering to a cell a PCSK9 RNAi agent that includes an antisense strand comprising the sequence of any of the sequences in Tables 2, 3, 5C, 7A, or 8 and a sense strand that comprises any of the sequences in Tables 2, 4, 5C, 7B, or 8 that is at least partially complementary to the antisense strand. In some embodiments, disclosed herein are methods of inhibiting expression of a PCSK9 gene in a cell, wherein the methods include administering a PCSK9 RNAi agent that includes a sense strand that comprises any of the sequences in Tables 2, 4, 5C, 7B, or 8, and an antisense strand that includes the sequence of any of the sequences in Tables 2, 3, 5C, 7A, or 8 that is at least partially complementary to the sense strand.

The use of PCSK9 RNAi agents provides methods for therapeutic (including prophylactic) treatment of diseases/disorders associated with including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease. The described PCSK9 RNAi agents mediate RNA interference to inhibit the expression of one or more genes necessary for production of PCSK9 protein. PCSK9 RNAi agents can also be used to treat or prevent various diseases, disorders, or conditions, including including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease. Furthermore, compositions for delivery of PCSK9 RNAi agents to liver cells, and specifically to hepatocytes, in vivo, are described.

### Cells, Tissues, Organs, and Non-Human Organisms

Cells, tissues, organs, and non-human organisms that include at least one of the PCSK9 RNAi agents described herein are contemplated. The cell, tissue, organ, or non-human organism is made by delivering the RNAi agent to the cell, tissue, organ or non-human organism.

### Additional Illustrative Embodiments

Provided here are certain additional illustrative embodiments of the disclosed invention. These embodiments are illustrative only and do not limit the scope of the present disclosure or of the claims attached hereto.
Embodiment 1. An RNAi agent for inhibiting expression of a PCSK9 gene, comprising:
   an antisense strand wherein nucleotides 1-21 of the antisense strand comprise nucleotides 1-21 of the antisense strand sequences of Table 2, Table 3, Table 5C, Table 7A, or Table 8; and a sense strand comprising a nucleotide sequence that is at least partially complementary to the antisense strand,
   wherein all or substantially all of the nucleotides of the antisense strand and/or the sense strand are modified nucleotides, and the RNAi agent is linked to a targeting ligand that comprises N-acetyl-galactosamine.
Embodiment 2. An RNAi agent for inhibiting expression of a PCSK9 gene, wherein the sense strand comprises a nucleotide sequence of at least 15 contiguous nucleotides differing by 0 or 1 nucleotides from 15 contiguous nucleotides of any one of the sense strand sequences of Table 2, Table 4, Table 5C, Table 7B, or Table 8, and wherein the sense strand has a region of at least 85% complementarity over at least 15 contiguous nucleotides to the antisense strand.
Embodiment 3. The RNAi agent of any one of Embodiments 1-2, wherein at least one nucleotide of the RNAi agent includes a modified intemucleoside linkage.
Embodiment 4. The RNAi agent of any one of Embodiments 1-3, wherein the modified nucleotides are independently selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.
Embodiment 5. The RNAi agent of Embodiment 4, wherein all or substantially all of the modified nucleotides are 2′-O-methyl nucleotides, 2′-fluoro nucleotides, or combinations thereof.
Embodiment 6. The RNAi agent of any one of Embodiments 1-5, wherein the antisense strand consists of or consists essentially of the nucleotide sequence of any one of the modified antisense strand sequences of Table 3, Table 5C, Table 7A, or Table 8.
Embodiment 7. The RNAi agent of any one of Embodiments 1-6, wherein the sense strand consists of, consists essentially of, or comprises the nucleotide sequence of any of the modified sense strand sequences of Table 4, Table 5C, Table 7B, or Table 8.
Embodiment 8. The RNAi agent of Embodiment 1, wherein the antisense strand comprises the nucleotide sequence of any one of the modified sequences of Table 3, Table 5C, Table 7A, or Table 8, and the sense strand comprises the nucleotide sequence of any one of the modified sequences of Table 4, Table 5C, Table 7B, or Table 8.
Embodiment 9. The RNAi agent of any one of Embodiments 1-8, wherein the targeting ligand comprises:

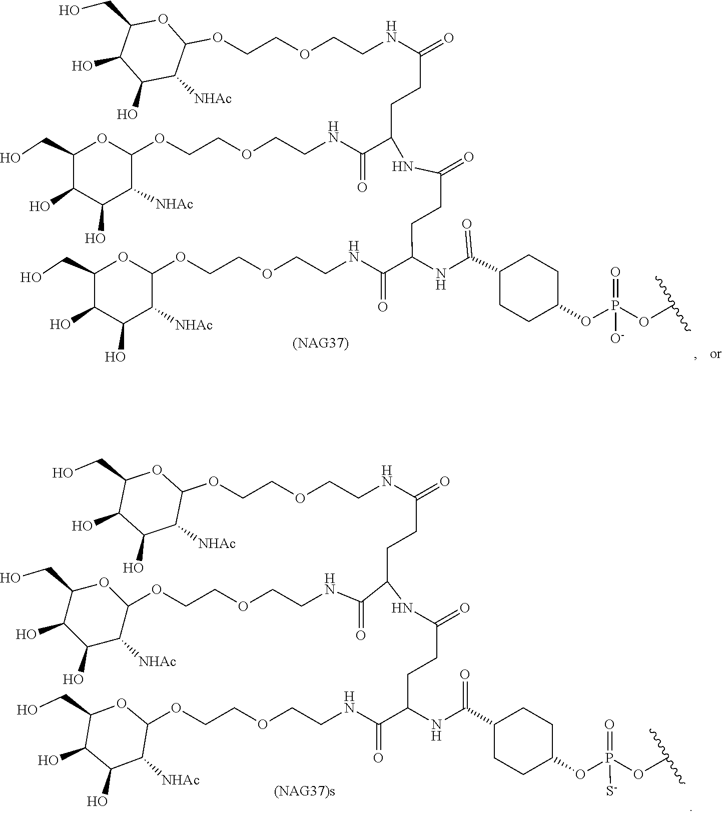

Embodiment 10. The RNAi agent of any one of Embodiments 1-9, wherein the targeting ligand is linked to the sense strand.
Embodiment 11. The RNAi agent of Embodiment 10, wherein the targeting ligand is linked to the 5′ terminal end of the sense strand.
Embodiment 12. The RNAi agent of any one of Embodiments 1-11, wherein the sense strand is between 15 and 30 nucleotides in length, and the antisense strand is between 21 and 30 nucleotides in length.
Embodiment 13. The RNAi agent of Embodiment 12, wherein the sense strand and the antisense strand are each between 21 and 27 nucleotides in length.
Embodiment 14. The RNAi agent of Embodiment 13, wherein the sense strand and the antisense strand are each between 21 and 24 nucleotides in length.
Embodiment 15. The RNAi agent of Embodiment 14, wherein the sense strand and the antisense strand are each 21 nucleotides in length.
Embodiment 16. The RNAi agent of any one of Embodiments 1-15, wherein the RNAi agent has two blunt ends.
Embodiment 17. The RNAi agent of any one of Embodiments 1-16, wherein the sense strand comprises one or two terminal caps.
Embodiment 18. The RNAi agent of any one of Embodiments 1-17, wherein the sense strand comprises one or two inverted abasic residues.
Embodiment 19. The RNAi agent of Embodiment 1, wherein the RNAi agent is comprised of a sense strand and an antisense strand that form a duplex sequence of any of the duplexes set forth in Table 5A, 5B, 5C, or 8.
Embodiment 20. The RNAi agent of any of Embodiments 1-19, wherein the RNAi agent is a pharmaceutically acceptable salt.
Embodiment 21. The RNAi agent of Embodiment 20, wherein the RNAi agent is a sodium salt.
Embodiment 22. A composition comprising the RNAi agent of any one of Embodiments 1-21, wherein the composition comprises a pharmaceutically acceptable excipient.
Embodiment 23. The composition of Embodiment 22, wherein the pharmaceutically acceptable excipient is a sodium phosphate buffer.
Embodiment 24. The composition of Embodiment 22, wherein the pharmaceutically acceptable excipient is isotonic saline or water for injection.
Embodiment 25. A method for inhibiting expression of a PCSK9 gene in a hepatocyte cell, the method comprising introducing into a cell an effective amount of an RNAi agent of any one of Embodiments 1-21 or the composition of any one of Embodiments 22-24.
Embodiment 26. The method of Embodiment 25, wherein the subject is a human subject.
Embodiment 27. The method of any one of Embodiments 25-26, wherein the PCSK9 mRNA levels are reduced by at least about 50% in the hepatocyte cell or in the subject.
Embodiment 28. The method of any one of Embodiments 25-27, wherein the PCSK9 protein levels are reduced by at least about 50% in the hepatocyte cell or in the subject.
Embodiment 29. A method of treating a PCSK9-related disease, disorder, or symptom, the method comprising administering to a human subject in need thereof a therapeutically effective amount of the composition of any one of Embodiments 22-24.
Embodiment 30. The method of Embodiment 29, wherein the disease is including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease.
Embodiment 31. The method of any one of Embodiments 25-30, wherein the level of serum PCSK9 protein is decreased in the subject.
Embodiment 32. The method of any one of Embodiments 25-31, wherein the RNAi agent is administered to a human subject at a dose of about 0.05 mg/kg to about 5.0 mg/kg of body weight of the human subject.
Embodiment 33. Use of the RNAi agent of any one of Embodiments 1-21 or the composition according to any one of Embodiments 22-24, for the treatment of a disease, disorder, or symptom that is mediated at least in part by a reduction in PCSK9 gene expression.
Embodiment 34. Use according to Embodiment 33, wherein the disease is including hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease.
Embodiment 35. Use of the RNAi agent of any one of Embodiments 1-21 or the composition according to any one of Embodiments 22-24, for the preparation of a pharmaceutical composition for treating a disease, disorder, or symptom that is mediated at least in part by a reduction in PCSK9 gene expression.
Embodiment 36. Use according to any one of Embodiments 33 to 35, wherein the RNAi agent is administered to a human subject at a dose of about 0.05 mg/kg to about 5.0 mg/kg of body weight of the human subject.

The above provided embodiments and items are now illustrated with the following, non-limiting examples.

### EXAMPLES

### Example 1. Synthesis of PCSK9 RN4i Agents

PCSK9 RNAi agent duplexes shown in Tables 5A, 5B, 5C, and 8 above, were synthesized in accordance with the following general procedures: A. Synthesis.

The sense and antisense strands of the RNAi agents were synthesized according to phosphoramidite technology on solid phase used in oligonucleotide synthesis. Such standard synthesis is generally known in the art. Depending on the scale, either a MerMade96E® (Bioautomation), a MerMadel2® (Bioautomation), or an OP Pilot 100 (GE Healthcare) was used. Syntheses were performed on a solid support made of controlled pore glass (CPG, 500 Å or 600A, obtained from Prime Synthesis, Aston, PA, USA). The monomer positioned at the 3′ end of the respective strand was attached to the solid support as a starting point for synthesis. All RNA and 2′-modified RNA phosphoramidites were purchased from Thermo Fisher Scientific (Milwaukee, WI, USA) or Hongene Biotech (Shanghai, PRC). The 2′-O-methyl phosphoramidites included the following: (5′-O-dimethoxytrityl-N⁶-(benzoyl)-2′-O-methyl-adenosine-3′-O-(2-cyanoethyl-N,N-diisopropylamino)phosphoramidite, 5′-O-dimethoxy-trityl-N⁴-(acetyl)-2′-O-methyl-cytidine-3′-O-(2-cyanoethyl-N,N-diisopropyl-amino)phosphoramidite, (5′-O-dimethoxytrityl-N²-(isobutyryl)-2′-O-methyl-guanosine-3′-O-(2-cyanoethyl-N,N-diisopropylamino)phosphoramidite, and 5′-O-dimethoxytrityl-2′-O-methyl-uridine-3′-O-(2-cyanoethyl-N,N-diisopropylamino)phosphoramidite. The 2′-deoxy-2′-fluoro-phosphoramidites carried the same protecting groups as the 2′-O-methyl amidites. 5′-(4,4′-Dimethoxytrityl)-2′,3′-seco-uridine, 2′-benzoyl-3′-[(2- cyanoethyl)-(N,N- diisopropyl)]-phosphoramidite was also purchased from Thermo Fisher Scientific or Hongene Biotech. 5′-dimethoxytrityl-2′-O-methyl-inosine-3′-O-(2-cyanoethyl-N,N-diisopropylamino)phosphoramidites were purchased from Glen Research (Virginia) or Hongene Biotech. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)). The inverted abasic (3′-O-dimethoxytrityl-2′-deoxyribose-5′-O-(2-cyanoethyl-N,N-diisopropylamino)phosphoramidites were purchased from ChemGenes (Wilmington, MA, USA) or SAFC (St Louis, MO, USA). 5′-O-dimethoxytrityl-N²,N⁶-(phenoxyacetate)-2′-O-methyl-diaminopurine-3′-O-(2-cyanoethyl-N,N-diisopropylamino)phosphoramidites were obtained from ChemGenes or Hongene Biotech.

Targeting ligand-containing phosphoramidites were dissolved in anhydrous dichloromethane or anhydrous acetonitrile (50 mM), while all other amidites were dissolved in anhydrous acetonitrile (50 mM), or anhydrous dimethylformamide and molecular sieves (3A) were added. 5-Benzylthio-1H-tetrazole (BTT, 250 mM in acetonitrile), 5-Ethylthio-1H-tetrazole (ETT, 250 mM in acetonitrile), or 4,5-dicyanoimidazole (DCI) was used as activator solution. Coupling times were 12 min (RNA), 15 min (targeting ligand), 90 sec (2′OMe), and 60 sec (2′F). In order to introduce phosphorothioate linkages, a 100 mM solution of 3-phenyl 1,2,4-dithiazoline-5-one (POS, obtained from PolyOrg, Inc., Leominster, MA, USA) in anhydrous Acetonitrile was employed. Each of the PCSK9 RNAi agent duplexes synthesized and tested in the following Examples utilized N-acetyl-galactosamine as “NAG” in the targeting ligand chemical structures represented in Table 6. (NAG37) and (NAG37)s targeting ligand phosphoramidite compounds can be synthesized in accordance with International Patent Application Publication No. WO 2018/044350 to Arrowhead Pharmaceuticals, Inc.

### B. Cleavage and Deprotection of Support Bound Oligoner.

After finalization of the solid phase synthesis, the dried solid support was treated with a 1:1 volume solution of 40 wt. % methylamine in water and 28% ammonium hydroxide solution (Aldrich) for 1.5 hours at 30° C. The solution was evaporated and the solid residue was reconstituted in water (see below).

### C. Purification.

Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 26/40 column packed with Sephadex G-25 fine with a running buffer of filtered DI water or 100 mM ammonium bicarbonate, pH 6.7 and 20% Acetonitrile.

### D. Annealing.

Complementary strands were mixed by combining equimolar RNA solutions (sense and antisense) in 1×Phosphate-Buffered Saline (Coming, Cellgro) to form the RNAi agents. Some RNAi agents were lyophilized and stored at −15 to −25° C. Duplex concentration was determined by measuring the solution absorbance on a UV-Vis spectrometer in 1×Phosphate-Buffered Saline. The solution absorbance at 260 nm was then multiplied by a conversion factor and the dilution factor to determine the duplex concentration. The conversion factor used was either 0.050 mg/(mL-cm) or was calculated from an experimentally determined extinction coefficient.

### Example 2. hPCSK9 AAV Mouse Model

To evaluate certain PCSK9 RNAi agents, a human PCSK9 (hPCSK9) AAV (Adeno-associated virus) mouse model was used. Six- to eight-week-old male C57BL/6 mice were transduced with the hPCSK9 AAV serotype 8, administered at least 14 days prior to administration of a PCSK9 RNAi agent or control. The genome of the hPCSK9 AAV contains the 291-3637 region (ORF plus 3′-UTR region) of the human PCSK9 cDNA sequence (GenBank NM_174936.4 (SEQ ID NO:1)), driven by a liver specific promoter. 4E12 to 1E13 GC/kg of the virus in PBS in a total volume of 10 mL/kg animal's body weight was injected into mice via the tail vein to create hPCSK9 AAV model mice. Inhibition of expression of hPCSK9 in liver by a PCSK9 RNAi agent results in reduction in serum levels of hPCSK9 protein, which is measured. Prior to administration of a treatment (between day -7 and day 1 pre-dose), hPCSK9 protein levels in serum were measured by the Human Proprotein Convertase 9/PCSK9 Quantikine ELISA Kit (Biotechne), and the mice were grouped according to average hPCSK9 protein levels.

Mice were anesthetized with 2-3% isoflurane and blood samples were collected from the submandibular area into serum separation tubes (Sarstedt A_{G} & Co., Numbrecht, Germany). Blood was allowed to coagulate at ambient temperature for 20 min. The tubes were centrifuged at 8,000×g for 3 min to separate the serum and stored at 4° C. Serum was collected and measured by the Human Proprotein Convertase 9/PCSK9 Quantikine ELISA Kit according to the manufacturer's instructions. Serum hPCSK9 levels for each animal can be normalized to the control group of mice injected with vehicle control in order to account for the non-treatment related shift in hPCSK9 expression with this model. To do so, first, the hPCSK9 level for each animal at a time point was divided by the pre-treatment level of expression in that animal (Day 1) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal vehicle control group. Alternatively, the serum hPCSK9 levels for each animal was assessed by normalizing to pre-treatment levels only.

### Example 3. In Vivo Testing of PCSK9 RNAi Agents in hPCSK9 AAV Mice

The hPCSK9 AAV mouse model described in Example 2, above, having the hPCSK9 AAV containing the 291-3637 region of the human PCSK9 cDNA sequence, was used. At day 1, each mouse was given a single subcutaneous administration of 250 μl/25 g animal weight containing 1.0 mg/kg (mpk) of a PCSK9 RNAi agent formulated in isotonic saline, or vehicle control (isotonic saline with no RNAi agent), according to the following Table 9:

TABLE 9
Targeted Positions and Dosing Groups of Example 3.

| | Targeted Gene | | |
|---|---|---|---|
| | Position | | |
| | (within SEQ | RNAi Agent and | |
| Group | ID NO: 1) | Dose | Dosing Regimen |
| 1 | N/A | Saline | Single injection on day 1 |
| | | (no RNAi agent) | |
| 2 | 614 | 1.0 mg/kg AD07404 | Single injection on day 1 |
| 3 | 604 | 1.0 mg/kg AD07690 | Single injection on day 1 |
| 4 | 605 | 1.0 mg/kg AD07691 | Single injection on day 1 |
| 5 | 612 | 1.0 mg/kg AD07692 | Single injection on day 1 |
| 6 | 3468 | 1.0 mg/kg AD07418 | Single injection on day 1 |
| 7 | 3501 | 1.0 mg/kg AD07419 | Single injection on day 1 |
| 8 | 3502 | 1.0 mg/kg AD07420 | Single injection on day 1 |
| 9 | 3531 | 1.0 mg/kg AD07421 | Single injection on day 1 |
| 10 | 3532 | 1.0 mg/kg AD07422 | Single injection on day 1 |
| 11 | 3536 | 1.0 mg/kg AD07423 | Single injection on day 1 |
| 12 | 3301 | 1.0 mg/kg AD07710 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD07404 (Group 2) included nucleotide sequences that were designed to inhibit expression of a PCSK9 gene at position 614 of the gene; the PCSK9 RNAi agent AD07690 (Group 3) included nucleotide sequences that were designed to inhibit expression of a PCKS9 gene at position 604 of the gene; the PCSK9 RNAi agent AD07691 (Group 4) included nucleotide sequences that were designed to inhibit expression of a PCSK9 gene at position 605 of the gene; the PCSK9 RNAi agent AD07692 (Group 5) included nucleotide sequences that were designed to inhibit expression of a PCSK9 gene at position 612 of the gene; the PCSK9 RNAi agent AD07418 (Group 6) included nucleotide sequences that were designed to inhibit expression of a PCSK9 gene at position 3468 of the gene; the PCSK9 RNAi agent AD07419 (Group 7) included nucleotide sequences that were designed to inhibit expression of a PCSK9 gene at position 3501 of the gene; the PCSK9 RNAi agent AD07420 (Group 8) included nucleotide sequences that were designed to inhibit expression of a PCSK9 gene at position 3502 of the gene; the PCSK9 RNAi agent AD07421 (Group 9) included nucleotide sequences that were designed to inhibit expression of a PCSK9 gene at position 3531 of the gene; the PCSK9 RNAi agent AD07422 (Group 10) included nucleotide sequences that were designed to inhibit expression of a PCSK9 gene at position 3532 of the gene; the PCSK9 RNAi agent AD07423 (Group 11) included nucleotide sequences that were designed to inhibit expression of a PCSK9 gene at position 3536 of the gene; the PCSK9 RNAi agent AD07710 (Group 12) included nucleotide sequences that were designed to inhibit expression of a PCSK9 gene at position 3301 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, day 22, and day 29, and PCSK9 expression levels were determined pursuant to the procedure set forth in Example 2, above. Data from the experiment are shown in the following Table 10:

TABLE 10
Average Serum PCSK9 Protein Levels Normalized to Pre-
Treatment & Control in hPCSK9 AAV Mice from Example 3.

| | Day 8 | | Day 15 | | Day 22 | | Day 29 | |
|---|---|---|---|---|---|---|---|---|
| | Avg | Std Dev | Avg | Std Dev | Avg | Std Dev | Avg | Std Dev |
| Group ID | PCSK9 | (+/−) | PCSK9 | (+/−) | PCSK9 | (+/−) | PCSK9 | (+/−) |
| Group 1 (Saline vehicle) | 1.000 | 0.044 | 1.000 | 0.089 | 1.000 | 0.035 | 1.000 | 0.143 |
| Group 2 1.0 mg/kg AD07404 | 0.784 | 0.062 | 0.944 | 0.047 | 0.922 | 0.107 | 0.890 | 0.145 |
| Group 3 1.0 mg/kg AD07690 | 0.956 | 0.029 | 1.245 | 0.075 | 1.133 | 0.075 | 1.117 | 0.102 |
| Group 4 1.0 mg/kg AD07691 | 1.077 | 0.089 | 1.148 | 0.124 | 1.173 | 0.054 | 1.168 | 0.139 |
| Group 5 1.0 mg/kg AD07692 | 0.923 | 0.064 | 1.156 | 0.068 | 1.081 | 0.088 | 1.066 | 0.110 |
| Group 6 1.0 mg/kg AD07418 | 0.518 | 0.087 | 0.610 | 0.131 | 0.543 | 0.172 | 0.651 | 0.153 |
| Group 7 1.0 mg/kg AD07419 | 0.802 | 0.025 | 1.008 | 0.156 | 0.901 | 0.176 | 1.040 | 0.217 |
| Group 8 1.0 mg/kg AD07420 | 0.782 | 0.077 | 0.759 | 0.111 | 0.769 | 0.125 | 0.813 | 0.076 |
| Group 9 1.0 mg/kg AD07421 | 0.596 | 0.069 | 0.690 | 0.119 | 0.771 | 0.139 | 0.845 | 0.161 |
| Group 10 1.0 mg/kg AD07422 | 0.872 | 0.162 | 0.801 | 0.126 | 0.680 | 0.055 | 0.728 | 0.124 |
| Group 11 1.0 mg/kg AD07423 | 0.472 | 0.062 | 0.411 | 0.095 | 0.472 | 0.075 | 0.537 | 0.128 |
| Group 12 1.0 mg/kg AD07710 | 0.868 | 0.178 | 0.993 | 0.283 | 1.049 | 0.078 | 1.012 | 0.205 |

As shown in Table 10, while a few of the PCSK9 RNAi agents tested showed little to no inhibitory activity compared to saline control, certain PCSK9 RNAi agents robustly silenced PCSK9 gene expression. In particular, a single dose of 1.0 mg/kg of AD07423 (targeting position 3536 of the PCSK9 gene) showed approximately 59% (0.411) PCSK9 protein reduction at Day 15.

### Example 4. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.5 mg/kg (mpk) of an PCSK9 RNAi agent formulated in isotonic saline, or vehicle control (isotonic saline with no RNAi agent), according to the following Table 11.

TABLE 11
Targeted Positions and Dosing Groups of Example 4.

| | Targeted Gene | | |
|---|---|---|---|
| | Position | | |
| | (within SEQ | RNAi Agent and | |
| Group | ID NO: 1) | Dose | Dosing Regimen |
| 1 | N/A | Saline | Single injection on day 1 |
| | | (no RNAi agent) | |
| 2 | 3536 | 1.5 mg/kg AD07423 | Single injection on day 1 |
| 3 | 3466 | 1.5 mg/kg AD07711 | Single injection on day 1 |
| 4 | 3490 | 1.5 mg/kg AD07712 | Single injection on day 1 |
| 5 | 3500 | 1.5 mg/kg AD07713 | Single injection on day 1 |
| 6 | 3534 | 1.5 mg/kg AD07714 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD07423 (Group 2) included nucleotide sequences that were designed to inhibit expression of a PCSK9 gene at position 3536 of the gene; the PCSK9 RNAi agent AD07711 (Group 3) included nucleotide sequences that were designed to inhibit expression of a PCKS9 gene at position 3466 of the gene; the PCSK9 RNAi agent AD07712 (Group 4) included nucleotide sequences that were designed to inhibit expression of a PCSK9 gene at position 3490 of the gene; the PCSK9 RNAi agent AD07713 (Group 5) included nucleotide sequences that were designed to inhibit expression of a PCSK9 gene at position 3500 of the gene; the PCSK9 RNAi agent AD07714 (Group 6) included nucleotide sequences that were designed to inhibit expression of a PCSK9 gene at position 3534 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, and day 15, and PCSK9 expression levels were determined pursuant to the procedure set forth in Example 2, above. Data from the experiment are shown in the following Table 12:

TABLE 12
Average Serum PCSK9 Protein Levels Normalized to Pre-
Treatment & Control in hPCSK9 AAV Mice from Example 4.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |

| | | | | |
|---|---|---|---|---|
| Group 1 (Saline vehicle) | 1.000 | 0.143 | 1.000 | 0.063 |
| Group 2 1.5 mg/kg AD07423 | 0.325 | 0.112 | 0.281 | 0.106 |
| Group 3 1.5 mg/kg AD07711 | 0.637 | 0.039 | 0.660 | 0.108 |
| Group 4 1.5 mg/kg AD07712 | 0.661 | 0.163 | 0.709 | 0.171 |
| Group 5 1.5 mg/kg AD07713 | 0.663 | 0.146 | 0.710 | 0.042 |
| Group 6 1.5 mg/kg AD07714 | 0.411 | 0.009 | 0.384 | 0.014 |

As shown in Table 12, each of the PCSK9 RNAi agents tested showed silencing of PCSK9 protein expression in the hPCSK9 AAV mouse model. In particular, a single dose of 1.5 mg/kg of PCSK9 RNAi agent AD07423 showed approximately 72% (0.281) inhibition at Day 15.

### Example 5. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.5 mg/kg (mpk) of an PCSK9 RNAi agent formulated in isotonic saline, or vehicle control (isotonic saline with no RNAi agent), according to the following Table 13.

TABLE 13
Targeted Positions and Dosing Groups of Example 5.

| | Targeted Gene | | |
|---|---|---|---|
| | Position | | |
| | (within SEQ | RNAi Agent and | |
| Group | ID NO: 1) | Dose | Dosing Regimen |
| 1 | N/A | Saline | Single injection on day 1 |
| | | (no RNAi agent) | |
| 2 | 3536 | 1.5 mg/kg AD07423 | Single injection on day 1 |
| 3 | 3536 | 1.5 mg/kg AD07911 | Single injection on day 1 |
| 4 | 3536 | 1.5 mg/kg AD07912 | Single injection on day 1 |
| 5 | 3536 | 1.5 mg/kg AD07913 | Single injection on day 1 |
| 6 | 3536 | 1.5 mg/kg AD07914 | Single injection on day 1 |
| 7 | 3536 | 1.5 mg/kg AD07915 | Single injection on day 1 |
| 8 | 3536 | 1.5 mg/kg AD07916 | Single injection on day 1 |
| 9 | 3536 | 1.5 mg/kg AD07917 | Single injection on day 1 |
| 10 | 3536 | 1.5 mg/kg AD07918 | Single injection on day 1 |
| 11 | 3536 | 1.5 mg/kg AD07919 | Single injection on day 1 |
| 12 | 3540 | 1.5 mg/kg AD07802 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agents in each of Groups 2 through 11 each included nucleotide sequences that were designed to inhibit expression of a PCSK9 gene at position 3536 of the gene; the PCSK9 RNAi agent AD07802 (Group 12) included nucleotide sequences that were designed to inhibit expression of a PCSK9 gene at position 3540 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, and day 15, and PCSK9 expression levels were determined pursuant to the procedure set forth in Example 2, above. Data from the experiment are shown in the following Table 14:

TABLE 14
Average Serum PCSK9 Protein Levels Normalized to Pre-
Treatment & Control in hPCSK9 AAV Mice from Example 5.

| | Day 8 | | Day 15 | |
|---|---|---|---|---|
| | Avg | Std Dev | Avg | Std Dev |
| Group ID | PCSK9 | (+/−) | PCSK9 | (+/−) |
| Group 1 (Saline vehicle) | 1.000 | 0.121 | 1.000 | 0.254 |
| Group 2 1.5 mg/kg AD07423 | 0.297 | 0.026 | 0.295 | 0.013 |
| Group 3 1.5 mg/kg AD07911 | 0.320 | 0.053 | 0.288 | 0.040 |
| Group 4 1.5 mg/kg AD07912 | 0.248 | 0.017 | 0.230 | 0.019 |
| Group 5 1.5 mg/kg AD07913 | 0.319 | 0.041 | 0.262 | 0.053 |
| Group 6 1.5 mg/kg AD07914 | 0.272 | 0.023 | 0.239 | 0.018 |
| Group 7 1.5 mg/kg AD07915 | 0.344 | 0.102 | 0.273 | 0.049 |
| Group 8 1.5 mg/kg AD07916 | 0.266 | 0.050 | 0.250 | 0.089 |
| Group 9 1.5 mg/kg AD07917 | 0.281 | 0.052 | 0.219 | 0.081 |
| Group 10 1.5 mg/kg AD07918 | 0.252 | 0.030 | 0.204 | 0.046 |
| Group 11 1.5 mg/kg AD07919 | 0.341 | 0.037 | 0.259 | 0.014 |
| Group 12 1.5 mg/kg AD07802 | 0.339 | 0.039 | 0.357 | 0.008 |

As shown in Table 14, each of the PCSK9 RNAi agents robustly silenced PCSK9 gene expression. Every PCSK9 RNAi agent targeting position 3536 of the PCSK9 gene (i.e., Groups 2-11) outperformed the PCSK9 RNAi agent of Group 12 (AD07802) that targeted position 3540. Groups 2-11 each had reductions at day 15 between 70% and 80% of PCSK9 protein normalized to pre-treatment and control.

### Example 6. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.0 mg/kg (mpk) of an PCSK9 RNAi agent formulated in isotonic saline, or vehicle control (isotonic saline with no RNAi agent), according to the following Table 15.

TABLE 15
Targeted Positions and Dosing Groups of Example 6.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 1.0 mg/kg AD07912 | Single injection on day 1 |
| 3 | 3536 | 1.0 mg/kg AD08105 | Single injection on day 1 |
| 4 | 3536 | 1.0 mg/kg AD08106 | Single injection on day 1 |
| 5 | 3536 | 1.0 mg/kg AD08107 | Single injection on day 1 |
| 6 | 3536 | 1.0 mg/kg AD08108 | Single injection on day 1 |
| 7 | 3536 | 1.0 mg/kg AD08109 | Single injection on day 1 |
| 8 | 3536 | 1.0 mg/kg AD08110 | Single injection on day 1 |
| 9 | 3536 | 1.0 mg/kg AD07423 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). All of the PCSK9 RNAi agents tested included nucleotide sequences that were designed to inhibit expression of a PCSK9 gene at position 3536 of the gene (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4) for Groups 1-8, and three (3) mice were tested (n=3) for Group 9. Serum was collected on day 1 (pre-treatment), day 8, day 15, and day 22, and PCSK9 expression levels were determined pursuant to the procedure set forth in Example 2, above. Data from the experiment are shown in the following Table 16:

TABLE 16
Average Serum PCSK9 Protein Levels Normalized to Pre-
Treatment & Control in hPCSK9 AAV Mice from Example 6.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.143 | 1.000 | 0.088 | 1.000 | 0.169 |
| Group 2 1.0 mg/kg AD07912 | 0.414 | 0.044 | 0.345 | 0.055 | 0.453 | 0.041 |
| Group 3 1.0 mg/kg AD08105 | 0.429 | 0.055 | 0.341 | 0.057 | 0.378 | 0.028 |
| Group 4 1.0 mg/kg AD08106 | 0.318 | 0.045 | 0.252 | 0.015 | 0.340 | 0.051 |
| Group 5 1.0 mg/kg AD08107 | 0.445 | 0.080 | 0.389 | 0.073 | 0.457 | 0.084 |
| Group 6 1.0 mg/kg AD08108 | 0.475 | 0.181 | 0.318 | 0.069 | 0.399 | 0.087 |
| Group 7 1.0 mg/kg AD08109 | 0.399 | 0.069 | 0.326 | 0.030 | 0.381 | 0.022 |
| Group 8 1.0 mg/kg AD08110 | 0.334 | 0.032 | 0.272 | 0.029 | 0.312 | 0.034 |
| Group 9 1.0 mg/kg AD07423 | 0.446 | 0.066 | 0.322 | 0.048 | 0.443 | 0.032 |

As shown in Table 16, each of the PCSK9 RNAi agents silenced PCSK9 gene expression.

### Example 7. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg (mpk) of an PCSK9 RNAi agent formulated in isotonic saline, or vehicle control (isotonic saline with no RNAi agent), according to the following Table 17.

TABLE 17
Targeted Positions and Dosing Groups of Example 7.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD08110 | Single injection on day 1 |
| 3 | 3536 | 0.3 mg/kg AD08574 | Single injection on day 1 |
| 4 | 3536 | 0.3 mg/kg AD08575 | Single injection on day 1 |
| 5 | 3536 | 0.3 mg/kg AD08576 | Single injection on day 1 |
| 6 | 3536 | 0.3 mg/kg AD08577 | Single injection on day 1 |
| 7 | 3536 | 0.3 mg/kg AD08578 | Single injection on day 1 |
| 8 | 3536 | 0.3 mg/kg AD08579 | Single injection on day 1 |
| 9 | 3536 | 0.3 mg/kg AD08580 | Single injection on day 1 |
| 10 | 3536 | 0.3 mg/kg AD08581 | Single injection on day 1 |
| 11 | 3536 | 0.3 mg/kg AD08106 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). Each of the PCSK9 RNAi agents tested included nucleotide sequences that were designed to inhibit expression of a PCSK9 gene at position 3536 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4) for Groups 1-10, and three (3) mice were tested (n=3) for Group 11. Serum was collected on day 1 (pre-treatment), day 8, day 15, day 22, day 29, and day 36, and PCSK9 expression levels were determined pursuant to the procedure set forth in Example 2, above. Data from the experiment are shown in the following Table 18:

TABLE 18
Average Serum PCSK9 Protein Levels Normalized to Pre-Treatment & Control in hPCSK9 AAV Mice from Example 7.

| | Day 8 | | Day 15 | | Day 22 | | Day 29 | | Day 36 | |
|---|---|---|---|---|---|---|---|---|---|---|
| | Avg | Std Dev | Avg | Std Dev | Avg | Std Dev | Avg | Std Dev | Avg | Std Dev |
| Group ID | PCSK9 | (+/−) | PCSK9 | (+/−) | PCSK9 | (+/−) | PCSK9 | (+/−) | PCSK9 | (+/−) |
| Group 1 (Saline vehicle) | 1.000 | 0.133 | 1.000 | 0.130 | 1.000 | 0.085 | 1.000 | 0.095 | 1.000 | 0.057 |
| Group 2 0.3 mg/kg AD08110 | 0.638 | 0.078 | 0.642 | 0.068 | 0.519 | 0.070 | 0.615 | 0.084 | 0.629 | 0.040 |
| Group 3 0.3 mg/kg AD08574 | 0.528 | 0.059 | 0.576 | 0.035 | 0.553 | 0.068 | 0.729 | 0.067 | 0.649 | 0.083 |
| Group 4 0.3 mg/kg AD08575 | 0.540 | 0.081 | 0.539 | 0.156 | 0.523 | 0.134 | 0.682 | 0.220 | 0.741 | 0.223 |
| Group 5 0.3 mg/kg AD08576 | 0.645 | 0.109 | 0.632 | 0.125 | 0.676 | 0.127 | 0.773 | 0.180 | 0.690 | 0.133 |
| Group 6 0.3 mg/kg AD08577 | 0.707 | 0.049 | 0.727 | 0.036 | 0.613 | 0.135 | 0.835 | 0.122 | 0.829 | 0.041 |
| Group 7 0.3 mg/kg AD08578 | 0.500 | 0.080 | 0.453 | 0.104 | 0.393 | 0.101 | 0.484 | 0.117 | 0.521 | 0.151 |
| Group 8 0.3 mg/kg AD08579 | 0.529 | 0.080 | 0.461 | 0.084 | 0.398 | 0.102 | 0.548 | 0.133 | 0.558 | 0.104 |
| Group 9 0.3 mg/kg AD08580 | 0.504 | 0.115 | 0.495 | 0.126 | 0.439 | 0.102 | 0.544 | 0.061 | 0.515 | 0.112 |
| Group 10 0.3 mg/kg AD08581 | 0.448 | 0.064 | 0.424 | 0.101 | 0.330 | 0.014 | 0.516 | 0.072 | 0.531 | 0.080 |
| Group 11 0.3 mg/kg AD08106 | 0.599 | 0.039 | 0.526 | 0.081 | 0.585 | 0.040 | 0.735 | 0.103 | 0.766 | 0.092 |

As shown in Table 18, each of the PCSK9 RNAi agents silenced PCSK9 gene expression, with reductions of PCSK9 protein compared to control continuing through day 36.

### Example 8. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg (mpk) of an PCSK9 RNAi agent formulated in isotonic saline, or vehicle control (isotonic saline with no RNAi agent), according to the following Table 19.

TABLE 19
Targeted Positions and Dosing Groups of Example 8.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD08110 | Single injection on day 1 |
| 3 | 3536 | 0.3 mg/kg AD08575 | Single injection on day 1 |
| 4 | 3536 | 0.3 mg/kg AD08581 | Single injection on day 1 |
| 5 | 3536 | 0.3 mg/kg AD08878 | Single injection on day 1 |
| 6 | 3536 | 0.3 mg/kg AD08879 | Single injection on day 1 |
| 7 | 3536 | 0.3 mg/kg AD08880 | Single injection on day 1 |
| 8 | 3531 | 1.0 mg/kg AD07421 | Single injection on day 1 |
| 9 | 3474 | 1.0 mg/kg AD08834 | Single injection on day 1 |
| 10 | 3486 | 1.0 mg/kg AD08835 | Single injection on day 1 |
| 11 | 3546 | 1.0 mg/kg AD08836 | Single injection on day 1 |
| 12 | 3547 | 1.0 mg/kg AD08837 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD08110 (Group 2) included nucleotide sequences that were designed to inhibit expression of a PCSK9 gene at position 3536 of the gene; the PCSK9 RNAi agent AD08575 (Group 3) included nucleotide sequences that were designed to inhibit expression of a PCKS9 gene at position 3536 of the gene; the PCSK9 RNAi agent AD08581 (Group 4) included nucleotide sequences that were designed to inhibit expression of a PCSK9 gene at position 3536 of the gene; the PCSK9 RNAi agent AD08878 (Group 5) included nucleotide sequences that were designed to inhibit expression of a PCSK9 gene at position 3536 of the gene; the PCSK9 RNAi agent AD08879 (Group 6) included nucleotide sequences that were designed to inhibit expression of a PCSK9 gene at position 3536 of the gene; the PCSK9 RNAi agent AD08880 (Group 7) included nucleotide sequences that were designed to inhibit expression of a PCSK9 gene at position 3536 of the gene; the PCSK9 RNAi agent AD07421 (Group 8) included nucleotide sequences that were designed to inhibit expression of a PCSK9 gene at position 3531 of the gene; the PCSK9 RNAi agent AD08834 (Group 9) included nucleotide sequences that were designed to inhibit expression of a PCSK9 gene at position 3474 of the gene; the PCSK9 RNAi agent AD08835 (Group 10) included nucleotide sequences that were designed to inhibit expression of a PCSK9 gene at position 3486 of the gene; the PCSK9 RNAi agent AD08836 (Group 11) included nucleotide sequences that were designed to inhibit expression of a PCSK9 gene at position 3546 of the gene; the PCSK9 RNAi agent AD08837 (Group 12) included nucleotide sequences that were designed to inhibit expression of a PCSK9 gene at position 3547 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 20:

TABLE 20
Average Serum PCSK9 Protein Levels Normalized to Pre-
Treatment & Control in hPCSK9 AAV Mice from Example 8.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.133 | 1.000 | 0.070 | 1.000 | 0.145 |
| Group 2 0.3 mg/kg AD08110 | 0.628 | 0.059 | 0.556 | 0.070 | 0.636 | 0.145 |
| Group 3 0.3 mg/kg AD08575 | 0.450 | 0.156 | 0.419 | 0.121 | 0.578 | 0.095 |
| Group 4 0.3 mg/kg AD08581 | 0.406 | 0.027 | 0.359 | 0.026 | 0.468 | 0.047 |
| Group 5 0.3 mg/kg AD08878 | 0.416 | 0.066 | 0.347 | 0.049 | 0.472 | 0.044 |
| Group 6 0.3 mg/kg AD08879 | 0.503 | 0.022 | 0.429 | 0.052 | 0.485 | 0.182 |
| Group 7 0.3 mg/kg AD08880 | 0.474 | 0.276 | 0.400 | 0.254 | 0.430 | 0.299 |
| Group 8 1.0 mg/kg AD07421 | 0.575 | 0.074 | 0.569 | 0.060 | 0.881 | 0.051 |
| Group 9 1.0 mg/kg AD08834 | 0.609 | 0.026 | 0.550 | 0.051 | 0.727 | 0.147 |
| Group 10 1.0 mg/kg AD08835 | 0.642 | 0.123 | 0.492 | 0.108 | 0.671 | 0.132 |
| Group 11 1.0 mg/kg AD08836 | 0.655 | 0.027 | 0.666 | 0.087 | 0.778 | 0.194 |
| Group 12 1.0 mg/kg AD08837 | 0.640 | 0.073 | 0.673 | 0.073 | 0.852 | 0.058 |

As shown in Table 20, at day 22 the top performers were Groups 2-7, which each have nucleotide sequences designed to inhibit expression at position 3536 of the PCSK9 gene. As noted above, Groups 2-7 were only dosed at 0.3 mg/kg, while Groups 8-12 were dosed at 1.0 mg/kg, indicating that the PCSK9 RNAi agents of Groups 2-7 are substantially more potent at inhibiting PCSK9 gene expression compared to the RNAi agents of Groups 8-12.

### Example 9. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.0 mg/kg (mpk) of an PCSK9 RNAi agent formulated in isotonic saline, or vehicle control (isotonic saline with no RNAi agent), according to the following Table 21.

TABLE 21
Targeted Positions and Dosing Groups of Example 9.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3531 | 1.0 mg/kg AD07421 | Single injection on day 1 |
| 3 | 1393 | 1.0 mg/kg AD08796 | Single injection on day 1 |
| 4 | 1859 | 1.0 mg/kg AD08838 | Single injection on day 1 |
| 5 | 3482 | 1.0 mg/kg AD08839 | Single injection on day 1 |
| 6 | 3465 | 1.0 mg/kg AD08840 | Single injection on day 1 |
| 7 | 887 | 1.0 mg/kg AD08841 | Single injection on day 1 |
| 8 | 3523 | 1.0 mg/kg AD08842 | Single injection on day 1 |
| 9 | 1543 | 1.0 mg/kg AD08843 | Single injection on day 1 |
| 10 | 1090 | 1.0 mg/kg AD08844 | Single injection on day 1 |
| 11 | 3548 | 1.0 mg/kg AD08845 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD07421 (Group 2) included nucleotide sequences that were designed to inhibit expression of a PCSK9 gene at position 3531 of the gene; the PCSK9 RNAi agent AD08796 (Group 3) included nucleotide sequences that were designed to inhibit expression of a PCKS9 gene at position 1393 of the gene; the PCSK9 RNAi agent AD08838 (Group 4) included nucleotide sequences that were designed to inhibit expression of a PCSK9 gene at position 1859 of the gene; the PCSK9 RNAi agent AD08839 (Group 5) included nucleotide sequences that were designed to inhibit expression of a PCSK9 gene at position 3482 of the gene; the PCSK9 RNAi agent AD08840 (Group 6) included nucleotide sequences that were designed to inhibit expression of a PCSK9 gene at position 3465 of the gene; the PCSK9 RNAi agent AD08841 (Group 7) included nucleotide sequences that were designed to inhibit expression of a PCSK9 gene at position 887 of the gene; the PCSK9 RNAi agent AD08842 (Group 8) included nucleotide sequences that were designed to inhibit expression of a PCSK9 gene at position 3523 of the gene; the PCSK9 RNAi agent AD08843 (Group 9) included nucleotide sequences that were designed to inhibit expression of a PCSK9 gene at position 1543 of the gene; the PCSK9 RNAi agent AD08844 (Group 10) included nucleotide sequences that were designed to inhibit expression of a PCSK9 gene at position 1090 of the gene; the PCSK9 RNAi agent AD08845 (Group 11) included nucleotide sequences that were designed to inhibit expression of a PCSK9 gene at position 3548 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 22:

TABLE 22
Average Serum PCSK9 Protein Levels Normalized to Pre-
Treatment & Control in hPCSK9 AAV Mice from Example 9.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.088 | 1.000 | 0.115 | 1.000 | 0.241 |
| Group 2 1.0 mg/kg AD07421 | 0.523 | 0.037 | 0.531 | 0.044 | 0.570 | 0.039 |
| Group 3 1.0 mg/kg AD08796 | 0.857 | 0.138 | 0.844 | 0.125 | 0.801 | 0.057 |
| Group 4 1.0 mg/kg AD08838 | 0.843 | 0.064 | 0.854 | 0.041 | 0.753 | 0.076 |
| Group 5 1.0 mg/kg AD08839 | 0.736 | 0.113 | 0.780 | 0.083 | 0.668 | 0.055 |
| Group 6 1.0 mg/kg AD08840 | 0.732 | 0.155 | 0.785 | 0.190 | 0.691 | 0.120 |
| Group 7 1.0 mg/kg AD08841 | 0.884 | 0.132 | 0.979 | 0.105 | 0.920 | 0.066 |
| Group 8 1.0 mg/kg AD08842 | 0.436 | 0.048 | 0.444 | 0.036 | 0.494 | 0.028 |
| Group 9 1.0 mg/kg AD08843 | 0.810 | 0.063 | 0.904 | 0.084 | 0.826 | 0.079 |
| Group 10 1.0 mg/kg AD08844 | 0.937 | 0.065 | 0.983 | 0.142 | 0.764 | 0.071 |
| Group 11 1.0 mg/kg AD08845 | 0.597 | 0.163 | 0.552 | 0.104 | 0.629 | 0.124 |

### Example 10. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.0 mg/kg (mpk) of an PCSK9 RNAi agent formulated in isotonic saline, or vehicle control (isotonic saline with no RNAi agent), according to the following Table 23.

TABLE 23
Targeted Positions and Dosing Groups of Example 10.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3531 | 1.0 mg/kg AD07421 | Single injection on day 1 |
| 3 | 3501 | 1.0 mg/kg AD08631 | Single injection on day 1 |
| 4 | 3501 | 1.0 mg/kg AD09027 | Single injection on day 1 |
| 5 | 3501 | 1.0 mg/kg AD09028 | Single injection on day 1 |
| 6 | 3501 | 1.0 mg/kg AD09029 | Single injection on day 1 |
| 7 | 3501 | 1.0 mg/kg AD09030 | Single injection on day 1 |
| 8 | 3501 | 1.0 mg/kg AD09031 | Single injection on day 1 |
| 9 | 3501 | 1.0 mg/kg AD09032 | Single injection on day 1 |
| 10 | 3501 | 1.0 mg/kg AD09033 | Single injection on day 1 |
| 11 | 3501 | 1.0 mg/kg AD09034 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD07421 (Group 2) included nucleotide sequences that were designed to inhibit expression of a PCSK9 gene at position 3531 of the gene; the PCSK9 RNAi agent AD08631 (Group 3) included nucleotide sequences that were designed to inhibit expression of a PCKS9 gene at position 3501 of the gene; the PCSK9 RNAi agent AD09027 (Group 4) included nucleotide sequences that were designed to inhibit expression of a PCKS9 gene at position 3501 of the gene; the PCSK9 RNAi agent AD09028 (Group 5) included nucleotide sequences that were designed to inhibit expression of a PCKS9 gene at position 3501 of the gene; the PCSK9 RNAi agent AD09029 (Group 6) included nucleotide sequences that were designed to inhibit expression of a PCKS9 gene at position 3501 of the gene; the PCSK9 RNAi agent AD09030 (Group 7) included nucleotide sequences that were designed to inhibit expression of a PCKS9 gene at position 3501 of the gene; the PCSK9 RNAi agent AD09031 (Group 8) included nucleotide sequences that were designed to inhibit expression of a PCKS9 gene at position 3501 of the gene; the PCSK9 RNAi agent AD09032 (Group 9) included nucleotide sequences that were designed to inhibit expression of a PCKS9 gene at position 3501 of the gene; the PCSK9 RNAi agent AD09033 (Group 10) included nucleotide sequences that were designed to inhibit expression of a PCKS9 gene at position 3501 of the gene; the PCSK9 RNAi agent AD09034 (Group 11) included nucleotide sequences that were designed to inhibit expression of a PCKS9 gene at position 3501 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 24:

TABLE 24
Average Serum PCSK9 Protein Levels Normalized to Pre-Treatment
& Control in hPCSK9 AAV Mice from Example 10.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.149 | 1.000 | 0.188 | 1.000 | 0.135 |
| Group 2 1.0 mg/kg AD07421 | 0.640 | 0.108 | 0.647 | 0.127 | 0.760 | 0.102 |
| Group 3 1.0 mg/kg AD08631 | 0.494 | 0.096 | 0.472 | 0.103 | 0.582 | 0.086 |
| Group 4 1.0 mg/kg AD09027 | 0.428 | 0.046 | 0.438 | 0.095 | 0.498 | 0.025 |
| Group 5 1.0 mg/kg AD09028 | 0.550 | 0.153 | 0.487 | 0.111 | 0.537 | 0.159 |
| Group 6 1.0 mg/kg AD09029 | 0.551 | 0.119 | 0.493 | 0.118 | 0.501 | 0.080 |
| Group 7 1.0 mg/kg AD09030 | 0.575 | 0.054 | 0.489 | 0.091 | 0.563 | 0.103 |
| Group 8 1.0 mg/kg AD09031 | 0.578 | 0.194 | 0.450 | 0.078 | 0.494 | 0.084 |
| Group 9 1.0 mg/kg AD09032 | 0.467 | 0.111 | 0.450 | 0.074 | 0.472 | 0.088 |
| Group 10 1.0 mg/kg AD09033 | 0.889 | 0.745 | 0.626 | 0.448 | 0.744 | 0.538 |
| Group 11 1.0 mg/kg AD09034 | 0.671 | 0.160 | 0.536 | 0.065 | 0.719 | 0.070 |

### Example 11. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 1.0 mg/kg (mpk) of an PCSK9 RNAi agent formulated in isotonic saline, or vehicle control (isotonic saline with no RNAi agent), according to the following Table 25.

TABLE 25
Targeted Positions and Dosing Groups of Example 11.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3531 | 1.0 mg/kg AD07421 | Single injection on day 1 |
| 3 | 3547 | 1.0 mg/kg AD08837 | Single injection on day 1 |
| 4 | 3547 | 1.0 mg/kg AD09038 | Single injection on day 1 |
| 5 | 3547 | 1.0 mg/kg AD09039 | Single injection on day 1 |
| 6 | 3547 | 1.0 mg/kg AD09040 | Single injection on day 1 |
| 7 | 3547 | 1.0 mg/kg AD09041 | Single injection on day 1 |
| 8 | 3486 | 1.0 mg/kg AD08835 | Single injection on day 1 |
| 9 | 3486 | 1.0 mg/kg AD09042 | Single injection on day 1 |
| 10 | 3486 | 1.0 mg/kg AD09043 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD07421 (Group 2) included nucleotide sequences that were designed to inhibit expression of a PCSK9 gene at position 3531 of the gene; the PCSK9 RNAi agent AD08837 (Group 3) included nucleotide sequences that were designed to inhibit expression of a PCKS9 gene at position 3547 of the gene; the PCSK9 RNAi agent AD09038 (Group 4) included nucleotide sequences that were designed to inhibit expression of a PCKS9 gene at position 3547 of the gene; the PCSK9 RNAi agent AD09039 (Group 5) included nucleotide sequences that were designed to inhibit expression of a PCKS9 gene at position 3547 of the gene; the PCSK9 RNAi agent AD09040 (Group 6) included nucleotide sequences that were designed to inhibit expression of a PCKS9 gene at position 3547 of the gene; the PCSK9 RNAi agent AD09041 (Group 7) included nucleotide sequences that were designed to inhibit expression of a PCKS9 gene at position 3547 of the gene; the PCSK9 RNAi agent AD08835 (Group 8) included nucleotide sequences that were designed to inhibit expression of a PCKS9 gene at position 3486 of the gene; the PCSK9 RNAi agent AD09042 (Group 9) included nucleotide sequences that were designed to inhibit expression of a PCKS9 gene at position 3486 of the gene; the PCSK9 RNAi agent AD09043 (Group 10) included nucleotide sequences that were designed to inhibit expression of a PCKS9 gene at position 3486 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 26:

TABLE 26
Average Serum PCSK9 Protein Levels Normalized to Pre-Treatment
& Control in hPCSK9 AAV Mice from Example 11.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.174 | 1.000 | 0.173 | 1.000 | 0.174 |
| Group 2 1.0 mg/kg AD07421 | 0.520 | 0.033 | 0.546 | 0.079 | 0.667 | 0.070 |
| Group 3 1.0 mg/kg AD08837 | 0.657 | 0.068 | 0.636 | 0.014 | 0.824 | 0.051 |
| Group 4 1.0 mg/kg AD09038 | 0.556 | 0.099 | 0.589 | 0.070 | 0.864 | 0.083 |
| Group 5 1.0 mg/kg AD09039 | 0.484 | 0.066 | 0.435 | 0.120 | 0.584 | 0.141 |
| Group 6 1.0 mg/kg AD09040 | 0.593 | 0.137 | 0.633 | 0.171 | 0.793 | 0.183 |
| Group 7 1.0 mg/kg AD09041 | 0.516 | 0.121 | 0.538 | 0.093 | 0.737 | 0.139 |
| Group 8 1.0 mg/kg AD08835 | 0.507 | 0.040 | 0.480 | 0.041 | 0.619 | 0.064 |
| Group 9 1.0 mg/kg AD09042 | 0.391 | 0.096 | 0.308 | 0.059 | 0.414 | 0.108 |
| Group 10 1.0 mg/kg AD09043 | 0.459 | 0.144 | 0.370 | 0.105 | 0.476 | 0.134 |

### Example 12. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg (mpk) of an PCSK9 RNAi agent formulated in isotonic saline, or vehicle control (isotonic saline with no RNAi agent), according to the following Table 27.

TABLE 27
Targeted Positions and Dosing Groups of Example 12.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD08106 | Single injection on day 1 |
| 3 | 3536 | 0.3 mg/kg AD07918 | Single injection on day 1 |
| 4 | 3536 | 0.3 mg/kg AD09131 | Single injection on day 1 |
| 5 | 3536 | 0.3 mg/kg AD09132 | Single injection on day 1 |
| 6 | 3536 | 0.3 mg/kg AD08879 | Single injection on day 1 |
| 7 | 3536 | 1.0 mg/kg AD08106 | Single injection on day 1 |
| 8 | 3536 | 1.0 mg/kg AD07918 | Single injection on day 1 |
| 9 | 3536 | 1.0 mg/kg AD09131 | Single injection on day 1 |
| 10 | 3536 | 1.0 mg/kg AD09132 | Single injection on day 1 |
| 11 | 3536 | 1.0 mg/kg AD08879 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD08106 (Groups 2 and 7) included nucleotide sequences that were designed to inhibit expression of a PCSK9 gene at position 3536 of the gene; the PCSK9 RNAi agent AD07918 (Groups 3 and 8) included nucleotide sequences that were designed to inhibit expression of a PCSK9 gene at position 3536 of the gene; the PCSK9 RNAi agent AD09131 (Groups 4 and 9) included nucleotide sequences that were designed to inhibit expression of a PCSK9 gene at position 3536 of the gene; the PCSK9 RNAi agent AD09132 (Groups 5 and 10) included nucleotide sequences that were designed to inhibit expression of a PCSK9 gene at position 3536 of the gene; the PCSK9 RNAi agent AD08879 (Groups 6 and 11) included nucleotide sequences that were designed to inhibit expression of a PCSK9 gene at position 3536 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 28:

TABLE 28
Average Serum PCSK9 Protein Levels Normalized to Pre-Treatment
& Control in hPCSK9 AAV Mice from Example 12.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.050 | 1.000 | 0.087 | 1.000 | 0.086 |
| Group 2 0.3 mg/kg AD08106 | 0.549 | 0.044 | 0.450 | 0.052 | 0.558 | 0.081 |
| Group 3 0.3 mg/kg AD07918 | 0.595 | 0.104 | 0.491 | 0.075 | 0.646 | 0.048 |
| Group 4 0.3 mg/kg AD09131 | 0.665 | 0.122 | 0.604 | 0.095 | 0.700 | 0.112 |
| Group 5 0.3 mg/kg AD09132 | 0.434 | 0.052 | 0.314 | 0.044 | 0.398 | 0.039 |
| Group 6 0.3 mg/kg AD08879 | 0.387 | 0.047 | 0.324 | 0.012 | 0.369 | 0.040 |
| Group 7 1.0 mg/kg AD08106 | 0.281 | 0.039 | 0.241 | 0.027 | 0.331 | 0.063 |
| Group 8 1.0 mg/kg AD07918 | 0.294 | 0.075 | 0.276 | 0.064 | 0.368 | 0.083 |
| Group 9 1.0 mg/kg AD09131 | 0.288 | 0.079 | 0.224 | 0.019 | 0.327 | 0.051 |
| Group 10 1.0 mg/kg AD09132 | 0.241 | 0.022 | 0.178 | 0.031 | 0.239 | 0.027 |
| Group 11 1.0 mg/kg AD08879 | 0.194 | 0.024 | 0.155 | 0.004 | 0.199 | 0.033 |

As shown in Table 28, each of the PCSK9 RNAi agents silenced PCSK9 gene expression. In particular, at day 22, the PCSK9 RNAi agent AD08870 was the most potent PCSK9 RNAi agent tested, having approximately 63% (0.369) PCSK9 protein reduction when administered a single dose of 0.3 mg/kg, and at a single dose of 1.0 mg/kg a reduction of approximately 80% (0.199), thereby exhibiting a dose response.

### Example 13. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg, 3.0 mg/kg (mpk) of an PCSK9 RNAi agent formulated in isotonic saline, or vehicle control (isotonic saline with no RNAi agent), according to the following Table 29.

TABLE 29
Targeted Positions and Dosing Groups of Example 13.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3523 | 0.3 mg/kg AD09059 | Single injection on day 1 |
| 3 | 3523 | 1.0 mg/kg AD09059 | Single injection on day 1 |
| 4 | 3523 | 3.0 mg/kg AD09059 | Single injection on day 1 |
| 5 | 3486 | 0.3 mg/kg AD09042 | Single injection on day 1 |
| 6 | 3486 | 1.0 mg/kg AD09042 | Single injection on day 1 |
| 7 | 3486 | 3.0 mg/kg AD09042 | Single injection on day 1 |
| 8 | 3531 | 0.3 mg/kg AD07421 | Single injection on day 1 |
| 9 | 3531 | 1.0 mg/kg AD07421 | Single injection on day 1 |
| 10 | 3531 | 3.0 mg/kg AD07421 | Single injection on day 1 |
| 11 | 3523 | 0.3 mg/kg AD08842 | Single injection on day 1 |
| 12 | 3523 | 1.0 mg/kg AD08842 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). The PCSK9 RNAi agent AD09059 (Groups 2, 3, and 4) included nucleotide sequences that were designed to inhibit expression of a PCSK9 gene at position 3523 of the gene; the PCSK9 RNAi agent AD09042 (Groups 5, 6, and 7) included nucleotide sequences that were designed to inhibit expression of a PCSK9 gene at position 3486 of the gene; the PCSK9 RNAi agent AD09131 (Groups 8, 9, and 10) included nucleotide sequences that were designed to inhibit expression of a PCSK9 gene at position 3531 of the gene; the PCSK9 RNAi agent AD08842 (Groups 11 and 12) included nucleotide sequences that were designed to inhibit expression of a PCSK9 gene at position 3523 of the gene. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4) for Groups 1-11, and three (3) mice were tested (n=3) for Group 12. Serum was collected on day 1 (pre-treatment), day 9, day 15, and day 22, and PCSK9 expression levels were determined pursuant to the procedure set forth in Example 2, above. Data from the experiment are shown in the following Table 30:

TABLE 30
Average Serum PCSK9 Protein Levels Normalized to Pre-Treatment
& Control in hPCSK9 AAV Mice from Example 13.

| | Day 9 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.092 | 1.000 | 0.092 | 1.000 | 0.077 |
| Group 2 0.3 mg/kg AD09059 | 0.558 | 0.016 | 0.619 | 0.061 | 0.652 | 0.039 |
| Group 3 1.0 mg/kg AD09059 | 0.234 | 0.070 | 0.280 | 0.068 | 0.304 | 0.089 |
| Group 4 3.0 mg/kg AD09059 | 0.138 | 0.013 | 0.169 | 0.010 | 0.170 | 0.015 |
| Group 5 0.3 mg/kg AD09042 | 0.677 | 0.040 | 0.753 | 0.103 | 0.715 | 0.105 |
| Group 6 1.0 mg/kg AD09042 | 0.512 | 0.075 | 0.459 | 0.086 | 0.325 | 0.047 |
| Group 7 3.0 mg/kg AD09042 | 0.225 | 0.028 | 0.134 | 0.020 | 0.096 | 0.008 |
| Group 8 0.3 mg/kg AD07421 | 0.913 | 0.153 | 1.053 | 0.107 | 1.103 | 0.132 |
| Group 9 1.0 mg/kg AD07421 | 0.596 | 0.128 | 0.641 | 0.137 | 0.634 | 0.071 |
| Group 10 3.0 mg/kg AD07421 | 0.269 | 0.072 | 0.352 | 0.052 | 0.348 | 0.050 |
| Group 11 0.3 mg/kg AD08842 | 0.736 | 0.067 | 0.825 | 0.098 | 0.843 | 0.092 |
| Group 12 1.0 mg/kg AD08842 | 0.398 | 0.049 | 0.422 | 0.039 | 0.484 | 0.080 |

### Example 14. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg, 3.0 mg/kg (mpk) of an PCSK9 RNAi agent formulated in isotonic saline, or vehicle control (isotonic saline with no RNAi agent), according to the following Table 31.

TABLE 31
Targeted Positions and Dosing Groups of Example 14.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD08110 | Single injection on day 1 |
| 3 | 3536 | 1.0 mg/kg AD08110 | Single injection on day 1 |
| 4 | 3536 | 3.0 mg/kg AD08110 | Single injection on day 1 |
| 5 | 3535 | 0.3 mg/kg AD09549 | Single injection on day 1 |
| 6 | 3535 | 1.0 mg/kg AD09549 | Single injection on day 1 |
| 7 | 3535 | 3.0 mg/kg AD09549 | Single injection on day 1 |
| 8 | 3535 | 0.3 mg/kg AC001566 | Single injection on day 1 |
| 9 | 3535 | 1.0 mg/kg AC001566 | Single injection on day 1 |
| 10 | 3535 | 3.0 mg/kg AC001566 | Single injection on day 1 |

The PCSK9 RNAi agent AD08110 included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information, including (NAG37)s ligand). The PCSK9 RNAi agent AD08110 (Groups 2, 3, and 4) included nucleotide sequences that were designed to inhibit expression of a PCSK9 gene at position 3536 of the gene; the duplex AD09549 (Groups 5, 6, and 7) included nucleotide sequences that were designed to inhibit expression of a PCSK9 gene at position 3535 of the gene; the duplex AC001566 (Groups 8, 9, and 10) included nucleotide sequences that were designed to inhibit expression of a PCSK9 gene at position 3535 of the gene. (See, e.g., SEQ ID NO:1 and Table 2 for the PCSK9 gene referenced).

The duplex AD09549 included the following modified nucleotide sequences: Antisense Strand (5′→3′): asGfsuuaCfaaaagcaAfaAfcagsgsu (SEQ ID NO: 466) Sense Strand (5′→3′): (NAG37)csusguuuUfGfCfuuuuguaacu (SEQ ID NO: 467) (See Table 6 for (NAG37) targeting ligand structure).

The duplex AC001566 included the following modified nucleotide sequences: Antisense Strand (5′→3′): asGfsuuaCfaaaagcaAfaAfcagsgsu (SEQ ID NO: 468) Sense Strand (5′→3′): csusguuuUfGfCfuuuuguaacu(C6-NH)-Tri-NAG-PEG4 (SEQ ID NO: 469). The sense strand was synthetized with a commercially-available C6-amino linker at the 3′-end to facilitate the linkage to a trivalent N-acetyl-galactosamine targeting ligand formulated with a terminal azide, resulting in a molecule with the following structure:

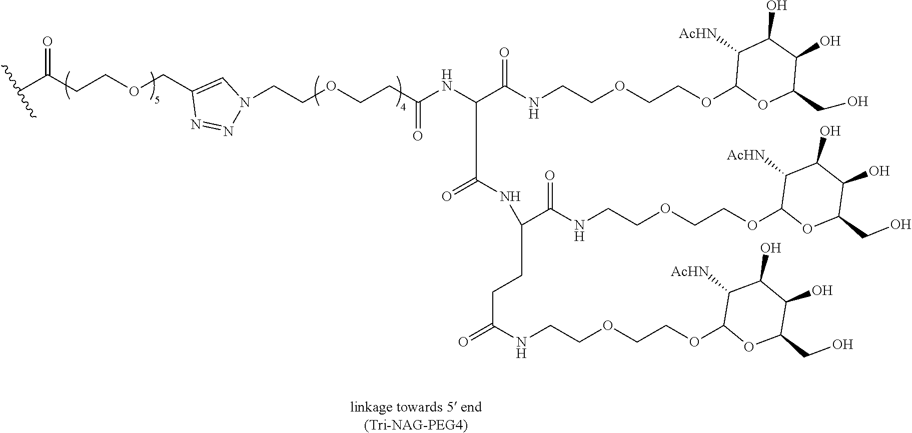

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 32:

TABLE 32
Average Serum PCSK9 Protein Levels Normalized to Pre-Treatment
& Control in hPCSK9 AAV Mice from Example 14.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.093 | 1.000 | 0.135 | 1.000 | 0.067 |
| Group 2 0.3 mg/kg AD08110 | 0.495 | 0.048 | 0.488 | 0.011 | 0.448 | 0.077 |
| Group 3 1.0 mg/kg AD08110 | 0.296 | 0.007 | 0.225 | 0.009 | 0.222 | 0.030 |
| Group 4 3.0 mg/kg AD08110 | 0.093 | 0.012 | 0.071 | 0.012 | 0.075 | 0.011 |
| Group 5 0.3 mg/kg AD09549 | 1.005 | 0.085 | 0.912 | 0.097 | 0.965 | 0.037 |
| Group 6 1.0 mg/kg AD09549 | 0.919 | 0.057 | 0.910 | 0.035 | 0.875 | 0.068 |
| Group 7 3.0 mg/kg AD09549 | 0.615 | 0.031 | 0.747 | 0.021 | 0.910 | 0.069 |
| Group 8 0.3 mg/kg AC001566 | 0.793 | 0.205 | 0.834 | 0.164 | 1.022 | 0.235 |
| Group 9 1.0 mg/kg AC001566 | 0.563 | 0.122 | 0.640 | 0.083 | 0.779 | 0.128 |
| Group 10 3.0 mg/kg AC001566 | 0.222 | 0.030 | 0.275 | 0.072 | 0.356 | 0.088 |

As shown in Table 32 above, the PCSK9 RNAi agent AD8110, which targeted position 3536 of the PCSK9 gene, showed superiority in inhibiting PCSK9 gene expression at all dosage levels over AD09549 and AC001566. For example, a 3.0 mg/kg dose of AD08110 reduced PCSK9 protein expression by more than 90% on days 8 (˜91% reduction), 15 (˜93% reduction), and 22 (˜93% reduction).

### Example 15. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg (mpk) of an PCSK9 RNAi agent formulated in isotonic saline, or vehicle control (isotonic saline with no RNAi agent), according to the following Table 33.

TABLE 33
Targeted Positions and Dosing Groups of Example 15.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD09327 | Single injection on day 1 |
| 3 | 3536 | 0.3 mg/kg AD09851 | Single injection on day 1 |
| 4 | 3536 | 0.3 mg/kg AD09852 | Single injection on day 1 |
| 5 | 3536 | 0.3 mg/kg AD09853 | Single injection on day 1 |
| 6 | 3536 | 0.3 mg/kg AD09854 | Single injection on day 1 |
| 7 | 3536 | 0.3 mg/kg AD09855 | Single injection on day 1 |
| 8 | 3536 | 1.0 mg/kg AD09327 | Single injection on day 1 |
| 9 | 3536 | 1.0 mg/kg AD09851 | Single injection on day 1 |
| 10 | 3536 | 1.0 mg/kg AD09852 | Single injection on day 1 |
| 11 | 3536 | 1.0 mg/kg AD09853 | Single injection on day 1 |
| 12 | 3536 | 1.0 mg/kg AD09854 | Single injection on day 1 |

Each of the PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). Each of the PCSK9 RNAi agents tested in this example included nucleotide sequences that were designed to inhibit expression of a PCSK9 gene at position 3536 of the gene, and were tested with a single injection at either 0.3 mg/kg or 1.0 mg/kg (AD09327 (Groups 2 and 8); AD09851 (Groups 3 and 9); AD09852 (Groups 4 and 10); AD09853 (Groups 5 and 11); AD09854 (Groups 6 and 12); except for Group 7, AD09855, which was only dosed at 0.3 mg/kg. (See, e.g., SEQ ID NO:1 and Table 2 for the PCSK9 gene referenced).

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 34:

TABLE 34
Average Serum PCSK9 Protein Levels Normalized to Pre-Treatment
& Control in hPCSK9 AAV Mice from Example 15.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.060 | 1.000 | 0.116 | 1.000 | 0.025 |
| Group 2 0.3 mg/kg AD09327 | 0.488 | 0.048 | 0.466 | 0.096 | 0.438 | 0.099 |
| Group 3 0.3 mg/kg AD09851 | 0.588 | 0.061 | 0.547 | 0.081 | 0.560 | 0.061 |
| Group 4 0.3 mg/kg AD09852 | 0.650 | 0.041 | 0.672 | 0.108 | 0.556 | 0.092 |
| Group 5 0.3 mg/kg AD09853 | 0.516 | 0.046 | 0.530 | 0.112 | 0.446 | 0.064 |
| Group 6 0.3 mg/kg AD09854 | 0.594 | 0.077 | 0.547 | 0.040 | 0.526 | 0.091 |
| Group 7 0.3 mg/kg AD09855 | 0.637 | 0.112 | 0.604 | 0.102 | 0.599 | 0.095 |
| Group 8 1.0 mg/kg AD09327 | 0.327 | 0.056 | 0.251 | 0.036 | 0.240 | 0.051 |
| Group 9 1.0 mg/kg AD09851 | 0.231 | 0.033 | 0.194 | 0.036 | 0.169 | 0.037 |
| Group 10 1.0 mg/kg AD09852 | 0.310 | 0.009 | 0.238 | 0.043 | 0.211 | 0.024 |
| Group 11 1.0 mg/kg AD09853 | 0.299 | 0.051 | 0.293 | 0.043 | 0.248 | 0.035 |
| Group 12 1.0 mg/kg AD09854 | 0.260 | 0.031 | 0.208 | 0.023 | 0.181 | 0.019 |

As shown in Table 34, each of the PCSK9 RNAi agents silenced PCSK9 gene expression, with AD09327 showing the greatest reductions in PCSK9 protein at the lowest (0.3 mg/kg) dose level.

### Example 16. In Vivo Testing of PCSK9 RNAi Agents in hPCSK9 AAV Mice

The hPCSK9 AAV mouse model described in Example 2, above, using the hPCSK9 AAV containing the 291-3637 region of the human PCSK9 cDNA sequence. At day 1, each mouse was given a single subcutaneous administration of 250 μl/25 g animal weight containing 0.3 mg/kg, 1.0 mg/kg, 3.0 mg/kg (mpk) of an PCSK9 RNAi agent formulated in isotonic saline, Leqvio® (Novartis Pharmaceuticals Corporation), or vehicle control (isotonic saline with no RNAi agent), according to the following Table 35.

TABLE 35
Targeted Positions and Dosing Groups of Example 16.

| | Targeted Gene Position | | |
|---|---|---|---|
| Group | (within SEQ ID NO: 1) | RNAi Agent and Dose | Dosing Regimen |
| 1 | N/A | Saline (no RNAi agent) | Single injection on day 1 |
| 2 | 3536 | 0.3 mg/kg AD09327-ETT | Single injection on day 1 |
| 3 | 3536 | 1.0 mg/kg AD09327-ETT | Single injection on day 1 |
| 4 | 3536 | 3.0 mg/kg AD09327-ETT | Single injection on day 1 |
| 5 | 3536 | 0.3 mg/kg AD09327-DCI | Single injection on day 1 |
| 6 | 3536 | 1.0 mg/kg AD09327-DCI | Single injection on day 1 |
| 7 | 3536 | 3.0 mg/kg AD09327-DCI | Single injection on day 1 |
| 8 | | 0.3 mg/kg Leqvio ® | Single injection on day 1 |
| 9 | | 1.0 mg/kg Leqvio ® | Single injection on day 1 |
| 10 | | 3.0 mg/kg Leqvio ® | Single injection on day 1 |
| 11 | 3536 | 0.3 mg/kg AD08879 | Single injection on day 1 |
| 12 | 3536 | 1.0 mg/kg AD08879 | Single injection on day 1 |

Leqvio® was acquired commercially. Leqvio®, or inclisiran, is long-acting RNA interference (RNAi) therapeutic agent that inhibits the synthesis of PCSK9, a target for lowering of low-density lipoprotein (LDL) cholesterol (Fitzgerald et al, A Highly Durable RNAi Therapeutic Inhibitor of PCSK9, N Engl J Med. 2017 Jan. 5; 376(1): 41-51).

Each of the PCSK9 RNAi agents tested included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the PCSK9 RNAi agents, including (NAG37)s ligand). Both PCSK9 RNAi agent AD09327 (Groups 2-7) and the PCSK9 RNAi agent AD08879 (Groups 11 and 12) included nucleotide sequences that were designed to inhibit expression of a PCSK9 gene at position 3536 of the gene. (See, e.g., SEQ ID NO:1 and Table 2 for the PCSK9 gene referenced). Groups designated with “ETT” and “DCI” denote PCSK9 RNAi agents synthesized using the respective ETT and DCI activator reagents.

The injections were performed between the skin and muscle (i.e. subcutaneous injections) into the loose skin over the neck and shoulder area. Four (4) mice in each group were tested (n=4). Serum was collected on day 1 (pre-treatment), day 8, day 15, and day 22, and PCSK9 expression levels were determined pursuant to the procedure set forth in Example 2, above. Data from the experiment are shown in the following Table 36:

TABLE 36
Average Serum PCSK9 Protein Levels Normalized to Pre-Treatment
& Control in hPCSK9 AAV Mice from Example 16.

| | Day 8 | | Day 15 | | Day 22 | |
|---|---|---|---|---|---|---|
| | Avg | Std Dev | Avg | Std Dev | Avg | Std Dev |
| Group ID | PCSK9 | (+/−) | PCSK9 | (+/−) | PCSK9 | (+/−) |
| Group 1 (Saline vehicle) | 1.000 | 0.168 | 1.000 | 0.241 | 1.000 | 0.193 |
| Group 2 0.3 mg/kg AD09327-ETT | 0.904 | 0.168 | 0.567 | 0.084 | 0.732 | 0.144 |
| Group 3 1.0 mg/kg AD09327-ETT | 0.425 | 0.212 | 0.304 | 0.088 | 0.382 | 0.138 |
| Group 4 3.0 mg/kg AD09327-ETT | 0.146 | 0.028 | 0.111 | 0.035 | 0.135 | 0.032 |
| Group 5 0.3 mg/kg AD09327-DCI | 0.710 | 0.135 | 0.569 | 0.068 | 0.677 | 0.052 |
| Group 6 1.0 mg/kg AD09327-DCI | 0.385 | 0.036 | 0.228 | 0.015 | 0.286 | 0.017 |
| Group 7 3.0 mg/kg AD09327-DCI | 0.209 | 0.056 | 0.120 | 0.028 | 0.140 | 0.026 |
| Group 8 0.3 mg/kg Leqvio ® | 1.146 | 0.123 | 0.875 | 0.123 | 1.066 | 0.112 |
| Group 9 1.0 mg/kg Leqvio ® | 0.682 | 0.047 | 0.531 | 0.073 | 0.679 | 0.051 |
| Group 10 3.0 mg/kg Leqvio ® | 0.456 | 0.011 | 0.346 | 0.080 | 0.585 | 0.031 |
| Group 11 0.3 mg/kg AD08879 | 0.811 | 0.072 | 0.503 | 0.051 | 0.536 | 0.073 |
| Group 12 1.0 mg/kg AD08879 | 0.408 | 0.049 | 0.235 | 0.078 | 0.282 | 0.029 |

As shown in Table 36, in the hPCSK9 AAV Mouse Model used in this particular study, PCSK9 RNAi agent AD09327 (whether -ETT or -DCI) showed improved inhibition of PCSK9 compared to the commercially available Leqvio® product across all dose levels.

### Example 17. In Vivo Testing of PCSK9 RNAi Agents in Cynomolgus Monkeys

PCSK9 RNAi agents AD07918 and AD09327 were evaluated in cynomolgus monkeys (cynos). On day 1, three female cynos for each group (n=3) were administered a subcutaneous injection of 0.3 mL/kg containing 3.0 mg/kg of the PCSK9 RNAi agent, formulated in isotonic saline. The subcutaneous dose was administered by syringe and needle in the mid-scapular region.

TABLE 37
Targeted Positions and Dosing Groups of Example 17.

| | Targeted Gene Position | | |
|---|---|---|---|
| Group | (within SEQ ID NO: 1) | RNAi Agent and Dose | Dosing Regimen (on day 1) |
| 1 | 3536 | 3.0 mg/kg AD07918 | Single subcutaneous injection |
| 2 | 3536 | 3.0 mg/kg AD09327 | Single subcutaneous injection |

The PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, 6, 7A, 7B, and 8 for specific modifications and structure information related to the PCSK9 RNAi agents, including (NAG37)s ligand). The PCSK9 RNAi agents included nucleotide sequences that were designed to inhibit expression of a human PCSK9 gene at position 3536. (See, e.g., SEQ ID NO:1).

The cynos were fasted overnight prior to each blood collection. Blood (approximately 5 mL) from each animal was collected from a femoral (or alternate) vein into tubes containing no anticoagulant (serum separator tubes) on days -14 (pre-dose), -7 (pre-dose), 1 (pre-dose), 8, 15, 22, 29, 43, 57, 71, and 85. Another vein not used for dosing may be used as alternative blood collection site. Pre-dose blood samples were collected from any available extra animal(s).

Serum collected from the cynos was quantified for human PCSK9 protein levels via ELISA, using Human Proprotein Convertase 9/PCSK9 Quantikine Elisa Kit (Catalog #DPC900, R&D Systems, Inc.). The PCSK9 expression levels were normalized to average pre-dose PCSK9 levels, shown below in Table 38.

TABLE 38
Average Serum PCSK9 Expression Levels Normalized to
Pre-Treatment in Cynomolgus Monkeys from Example 17.

| | |
|---|---|
| Group 1 | Group 2 |
| 3.0 mg/kg AD07918 | 3.0 mg/kg AD09327 |

| | Avg | Std Dev | Avg | Std Dev |
|---|---|---|---|---|
| | PCSK9 | +/− | PCSK9 | +/− |
| Day −14 | 1.079 | 0.110 | 0.888 | 0.239 |
| Day −7 | 0.913 | 0.025 | 0.750 | 0.063 |
| Day 1 | 1.008 | 0.134 | 1.362 | 0.216 |
| Day 8 | 0.653 | 0.308 | 0.541 | 0.078 |
| Day 15 | 0.451 | 0.211 | 0.277 | 0.096 |
| Day 22 | 0.290 | 0.149 | 0.183 | 0.047 |
| Day 29 | 0.381 | 0.184 | 0.275 | 0.059 |
| Day 43 | 0.298 | 0.176 | 0.207 | 0.147 |
| Day 57 | 0.420 | 0.093 | 0.258 | 0.097 |
| Day 71 | 0.424 | 0.084 | 0.210 | 0.111 |
| Day 85 | 0.664 | 0.067 | 0.509 | 0.327 |

The levels of low-density lipoprotein (LDL) and non-high-density lipoprotein (HDL) cholesterol were quantified, normalized to average pre-dose levels. This data is shown below in Table 39.

TABLE 39
Average Serum LDL and Non-HDL Cholesterol PCSK9 Levels Normalized
to Pre-Treatment & Control in Cynomolgus Monkeys from Example 17.

| | Group 1 | | | | Group 2 | | | |
|---|---|---|---|---|---|---|---|---|
| | 3.0 mg/kg AD07918 | | | | 3.0 mg/kg AD09327 | | | |
| | | | Non-HDL | | | | Non-HDL | |
| | LDL | SD +/− | Chol | SD +/− | LDL | SD +/− | Chol | SD +/− |
| Day −14 | 0.943 | 0.018 | 0.961 | 0.022 | 1.034 | 0.060 | 1.066 | 0.065 |
| Day −7 | 1.026 | 0.032 | 1.009 | 0.037 | 0.968 | 0.063 | 0.970 | 0.056 |
| Day 1 | 1.030 | 0.014 | 1.030 | 0.016 | 0.998 | 0.038 | 0.964 | 0.065 |
| Day 8 | 0.669 | 0.089 | 0.653 | 0.095 | 0.704 | 0.078 | 0.698 | 0.081 |
| Day 15 | 0.609 | 0.057 | 0.578 | 0.070 | 0.592 | 0.025 | 0.569 | 0.020 |
| Day 22 | 0.481 | 0.099 | 0.434 | 0.150 | 0.409 | 0.097 | 0.398 | 0.058 |
| Day 29 | 0.516 | 0.103 | 0.428 | 0.117 | 0.387 | 0.083 | 0.524 | 0.100 |
| Day 43 | 0.538 | 0.063 | 0.729 | 0.075 | 0.415 | 0.117 | 0.596 | 0.151 |
| Day 57 | 0.584 | 0.039 | 0.736 | 0.041 | 0.460 | 0.104 | 0.634 | 0.105 |
| Day 71 | 0.605 | 0.093 | 0.754 | 0.159 | 0.483 | 0.150 | 0.688 | 0.165 |
| Day 85 | 0.648 | 0.121 | 0.837 | 0.129 | 0.559 | 0.170 | 0.791 | 0.212 |

As shown in Table 38, both PCSK9 RNAi agents robustly silenced PCSK9 protein expression, with AD09327 being more potent and showing approximately 82% inhibition by AD09327 at Day 22 with a single 3.0 mg/kg dose, and further showing meaningful reductions of nearly 50% 85 days after the dose was administered. Further, as shown in Table 39, both PCSK9 RNAi agents robustly reduced LDL and non-HDL cholesterol levels, again with AD09327 being more robust and showing approximately 61% (0.387) LDL reduction at Day 29 and approximately 60% (0.398) non-HDL cholesterol reduction at day 22.

### Example 18. In Vivo Testing of PCSK9 RNAi Agents in Cynomolgus Monkeys

PCSK9 RNAi agents AD08879 and AD09327, Leqvio®, and AC001566 were evaluated in cynomolgus monkeys (cynos). On day 1 and day 29, three male cynos for each group (n=3) were administered a subcutaneous injection of 0.2 mL/kg containing 2.0 mg/kg of the PCSK9 RNAi agent, formulated in isotonic saline. The subcutaneous dose was administered by syringe and needle in the mid-scapular region.

TABLE 40
Targeted Positions and Dosing Groups of Example 18.

| | Targeted Gene Position | | Dosing Regimen |
|---|---|---|---|
| Group | (within SEQ ID NO: 1) | RNAi Agent and Dose | (on day 1 and day 29) |
| 1 | 3536 | 2.0 mg/kg AD08879 | 2 subcutaneous injections |
| 2 | 3536 | 2.0 mg/kg AD09327 | 2 subcutaneous injections |
| 3 | | 2.0 mg/kg Leqvio ® | 2 subcutaneous injections |
| 4 | 3535 | 2.0 mg/kg AC001566 | 2 subcutaneous injections |

The PCSK9 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, 6, 7A, 7B, and 8 for specific modifications and structure information related to the PCSK9 RNAi agents, including (NAG37)s ligand). The PCSK9 RNAi agents AD08879 and AD09327 included nucleotide sequences that were designed to inhibit expression of a human PCSK9 gene at position 3536. (See, e.g., SEQ ID NO:1).

AC001566 included the structure as described in Example 14, above.

The cynos were fasted overnight prior to each blood collection. Blood (approximately 5 mL) from each animal was collected from a femoral (or alternate) vein into tubes containing no anticoagulant (serum separator tubes) on days -8 (pre-dose), 1 (pre-dose), 8, 15, 22, 29, 36, 43, 50, 57, 71, 85, 99, and 113. Another vein not used for dosing may be used as alternative blood collection site. Pre-dose blood samples were collected from any available extra animal(s).

Serum collected from the cynos was quantified for human PCSK9 protein levels via ELISA, using Human Proprotein Convertase 9/PCSK9 Quantikine Elisa Kit (Catalog #DPC900, R&D Systems, Inc.). The PCSK9 expression levels were normalized to average pre-dose PCSK9 levels, shown below in Table 41.

TABLE 41
Average Serum PCSK9 Expression Levels Normalized to
Pre-Treatment in Cynomolgus Monkeys from Example 18.

| | Group 1 | | Group 2 | | Group 3 | | Group 4 | |
|---|---|---|---|---|---|---|---|---|
| | 2.0 mg/kg AD08879 | | 2.0 mg/kg AD09327 | | 2.0 mg/kg Leqvio ® | | 2.0 mg/kg AC001566 | |
| | Avg | Std Dev | Avg | Std Dev | Avg | Std Dev | Avg | Std Dev |
| | PCSK9 | +/− | PCSK9 | +/− | PCSK9 | +/− | PCSK9 | +/− |
| Day −7 | 1.004 | 0.058 | 1.178 | 0.117 | 1.091 | 0.197 | 1.049 | 0.026 |
| Day 1 | 0.996 | 0.058 | 0.822 | 0.117 | 0.909 | 0.197 | 0.951 | 0.026 |
| Day 8 | 0.530 | 0.114 | 0.467 | 0.166 | 0.518 | 0.063 | 0.681 | 0.122 |
| Day 15 | 0.611 | 0.177 | 0.330 | 0.113 | 0.307 | 0.119 | 1.019 | 0.103 |
| Day 22 | 0.552 | 0.243 | 0.426 | 0.276 | 0.605 | 0.282 | 1.076 | 0.195 |
| Day 29 | 0.492 | 0.085 | 0.393 | 0.322 | 0.357 | 0.140 | 1.064 | 0.216 |
| Day 36 | 0.806 | 0.419 | 0.450 | 0.264 | 0.438 | 0.147 | 1.621 | 0.345 |
| Day 43 | 0.506 | 0.111 | 0.386 | 0.218 | 0.483 | 0.295 | 1.821 | 0.546 |
| Day 50 | 0.881 | 0.271 | 0.514 | 0.325 | 0.686 | 0.343 | 1.590 | 0.098 |
| Day 57 | 1.166 | 0.609 | 0.661 | 0.350 | 0.739 | 0.338 | 1.676 | 1.136 |
| Day 64 | 0.403 | 0.099 | 0.426 | 0.129 | 0.415 | 0.110 | 0.945 | 0.316 |
| Day 71 | 0.409 | 0.134 | 0.360 | 0.082 | 0.393 | 0.115 | 0.997 | 0.386 |
| Day 78 | 0.384 | 0.029 | 0.430 | 0.115 | 0.496 | 0.049 | 1.271 | 0.185 |
| Day 85 | 0.325 | 0.099 | 0.445 | 0.205 | 0.326 | 0.060 | 1.017 | 0.510 |
| Day 92 | 0.302 | 0.054 | 0.331 | 0.107 | 0.346 | 0.050 | 2.358 | 0.239 |
| Day 99 | 0.329 | 0.087 | 0.355 | 0.136 | 0.338 | 0.033 | 1.223 | 0.554 |
| Day 106 | 0.450 | 0.136 | 0.490 | 0.227 | 0.469 | 0.081 | 1.417 | 0.350 |
| Day 113 | 0.506 | 0.026 | 0.584 | 0.286 | 0.512 | 0.115 | 1.737 | 0.728 |
| Day 122 | 0.682 | 0.136 | 0.474 | 0.188 | 0.491 | 0.101 | 1.647 | 0.462 |
| Day 127 | 0.597 | 0.078 | 0.694 | 0.338 | 0.559 | 0.059 | 1.578 | 0.223 |
| Day 134 | 0.831 | 0.084 | 0.806 | 0.474 | 0.763 | 0.101 | 2.012 | 0.615 |

The levels of low-density lipoprotein (LDL) and non-high-density lipoprotein (HDL) cholesterol were quantified, normalized to average pre-dose levels. This data is shown below in Table 42.

TABLE 42
Average Serum LDL and Non-HDL Cholesterol PCSK9 Levels Normalized
to Pre-Treatment & Control in Cynomolgus Monkeys from Example 18.

| | Group 1 | | Group 2 | | Group 3 | | Group 4 | |
|---|---|---|---|---|---|---|---|---|
| | 2.0 mg/kg | | 2.0 mg/kg | | 2.0 mg/kg | | 2.0 mg/kg | |
| | AD08879 | | AD09327 | | Leqvio ® | | AC001566 | |
| | | Non-HDL | | Non-HDL | | Non-HDL | | Non-HDL |
| | LDL | Chol | LDL | Chol | LDL | Chol | LDL | Chol |
| Day −7 | 1.035 | 1.028 | 0.957 | 0.952 | 0.951 | 0.942 | 0.996 | 1.008 |
| Day 1 | 0.965 | 0.972 | 1.043 | 1.048 | 1.049 | 1.058 | 1.004 | 0.992 |
| Day 8 | 0.630 | 0.681 | 0.657 | 0.689 | 0.699 | 0.767 | 0.631 | 0.622 |
| Day 15 | 0.486 | 0.596 | 0.571 | 0.605 | 0.671 | 0.738 | 0.575 | 0.679 |
| Day 22 | 0.473 | 0.527 | 0.495 | 0.540 | 0.486 | 0.629 | 0.651 | 0.703 |
| Day 29 | 0.580 | 0.659 | 0.585 | 0.563 | 0.580 | 0.615 | 0.828 | 0.794 |
| Day 36 | 0.516 | 0.622 | 0.531 | 0.537 | 0.605 | 0.671 | 0.732 | 0.801 |
| Day 43 | 0.600 | 0.651 | 0.445 | 0.542 | 0.537 | 0.630 | 0.686 | 0.780 |
| Day 50 | 0.584 | 0.719 | 0.462 | 0.523 | 0.519 | 0.685 | 0.730 | 0.838 |
| Day 57 | 0.612 | 0.757 | 0.535 | 0.596 | 0.549 | 0.670 | 0.816 | 0.871 |
| Day 64 | 0.549 | 0.860 | 0.442 | 0.664 | 0.540 | 0.841 | 0.594 | 0.788 |
| Day 71 | 0.502 | 0.647 | 0.487 | 0.588 | 0.496 | 0.669 | 0.640 | 0.711 |
| Day 78 | 0.459 | 0.557 | 0.469 | 0.536 | 0.464 | 0.639 | 0.608 | 0.675 |
| Day 85 | 0.606 | 0.647 | 0.477 | 0.504 | 0.641 | 0.667 | 0.874 | 0.874 |
| Day 92 | 0.621 | 0.693 | 0.541 | 0.608 | 0.552 | 0.658 | 0.780 | 0.891 |
| Day 99 | 0.598 | 0.618 | 0.579 | 0.635 | 0.551 | 0.601 | 0.933 | 0.897 |
| Day 106 | 0.556 | 0.628 | 0.639 | 0.629 | 0.590 | 0.713 | 0.892 | 0.903 |
| Day 113 | 0.607 | 0.656 | 0.594 | 0.626 | 0.626 | 0.676 | 0.923 | 0.952 |
| Day 122 | 0.544 | 0.733 | 0.540 | 0.644 | 0.524 | 0.697 | 0.773 | 0.831 |
| Day 127 | 0.663 | 0.773 | 0.642 | 0.700 | 0.599 | 0.678 | 0.956 | 1.021 |
| Day 134 | 0.574 | 0.800 | 0.589 | 0.746 | 0.614 | 0.802 | 0.808 | 0.957 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a PCSK9 gene, comprising:
an antisense strand wherein nucleotides 1-21 of the antisense strand comprise nucleotides 1-21 of the antisense strand sequences of Table 2, Table 3, Table 5C, Table 7A, or Table 8; and
a sense strand comprising a nucleotide sequence that is at least partially complementary to the antisense strand;
wherein all or substantially all of the nucleotides of the antisense strand and/or the sense strand are modified nucleotides, and the RNAi agent is linked to a targeting ligand that comprises N-acetyl-galactosamine.

2. The RNAi agent of claim 1, wherein the sense strand comprises a nucleotide sequence of at least 15 contiguous nucleotides differing by 0 or 1 nucleotides from 15 contiguous nucleotides of any one of the sense strand sequences of Table 2, Table 4, Table 5C, Table 7B, or Table 8, and wherein the sense strand has a region of at least 85% complementarity over at least 15 contiguous nucleotides to the antisense strand.

3. The RNAi agent of any one of claims 1-2, wherein at least one nucleotide of the RNAi agent includes a modified internucleoside linkage.

4. The RNAi agent of any one of claims 1-3, wherein the modified nucleotides are independently selected from the group consisting of 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

5. The RNAi agent of claim 4, wherein all or substantially all of the modified nucleotides are 2′-O-methyl nucleotides, 2′-fluoro nucleotides, or combinations thereof.

6. The RNAi agent of any one of claims 1-5, wherein the antisense strand consists of or consists essentially of the nucleotide sequence of any one of the modified antisense strand sequences of Table 3, Table 5C, Table 7A, or Table 8.

7. The RNAi agent of any one of claims 1-6, wherein the sense strand consists of, consists essentially of, or comprises the nucleotide sequence of any of the modified sense strand sequences of Table 4, Table 5C, Table 7B, or Table 8.

8. The RNAi agent of claim 1, wherein the antisense strand comprises the nucleotide sequence of any one of the modified sequences of Table 3, Table 5C, Table 7A, or Table 8, and the sense strand comprises the nucleotide sequence of any one of the modified sequences of Table 4, Table 5C, Table 7B, or Table 8.

9. The RNAi agent of any one of claims 1-8, wherein the targeting ligand comprises the structure: 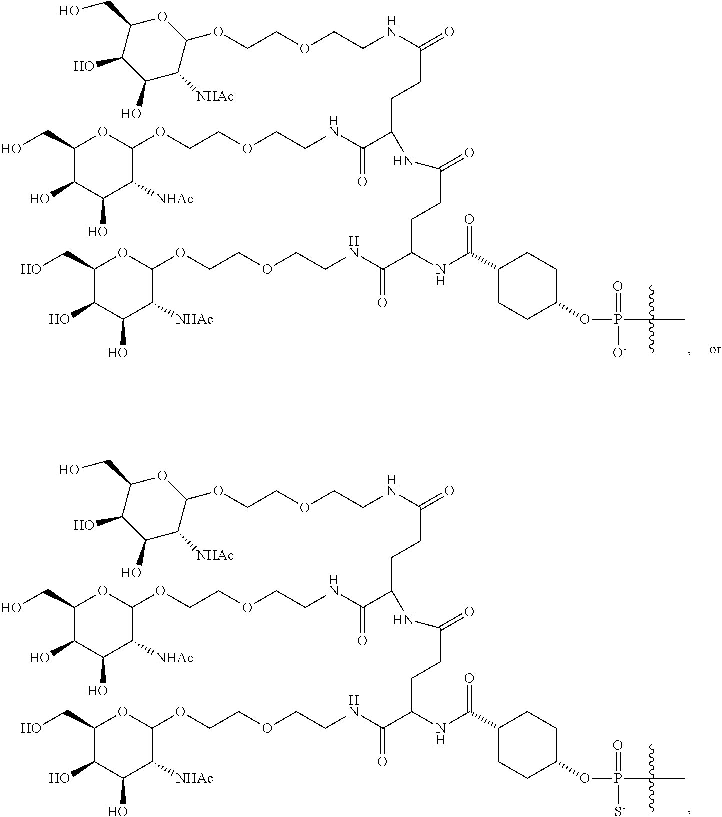
pharmaceutically acceptable salt thereof.

10. The RNAi agent of any one of claims 1-9, wherein the targeting ligand is linked to the sense strand.

11. The RNAi agent of claim 10, wherein the targeting ligand is linked to the 5′ terminal end of the sense strand.

12. The RNAi agent of any one of claims 1-11, wherein the sense strand is between 15 and 30 nucleotides in length, and the antisense strand is between 21 and 30 nucleotides in length.

13. The RNAi agent of claim 12, wherein the sense strand and the antisense strand are each between 21 and 27 nucleotides in length.

14. The RNAi agent of claim 13, wherein the sense strand and the antisense strand are each between 21 and 24 nucleotides in length.

15. The RNAi agent of claim 14, wherein the sense strand and the antisense strand are each 21 nucleotides in length.

16. The RNAi agent of any one of claims 1-15, wherein the RNAi agent has two blunt ends.

17. The RNAi agent of any one of claims 1-16, wherein the sense strand comprises one or two terminal caps.

18. The RNAi agent of any one of claims 1-17, wherein the sense strand comprises one or two inverted abasic residues.

19. The RNAi agent of claim 1, wherein the RNAi agent is comprised of a sense strand and an antisense strand that form a duplex sequence of any of the duplexes set forth in Table 5A, 5B, 5C, or 8.

20. The RNAi agent of any of claims 1-19, wherein the RNAi agent is a pharmaceutically acceptable salt.

21. The RNAi agent of claim 20, wherein the RNAi agent is a sodium salt.

22. The RNAi agent of any of claims 1-21, wherein the RNAi agent comprises an antisense strand comprising or consisting of the nucleotide sequence of SEQ ID NO:401, and a sense strand comprising or consisting of the nucleotide sequence of SEQ ID NO:430.

23. The RNAi agent of claim 22, wherein the RNAi agent comprises a modified antisense strand comprising or consisting of the nucleotide sequence of SEQ ID NO:309 and a modified sense strand comprising or consisting of the nucleotide sequence of SEQ ID NO:368.

24. A composition comprising the RNAi agent of any one of claims 1-23, wherein the composition comprises a pharmaceutically acceptable excipient.

25. The composition of claim 24, wherein the pharmaceutically acceptable excipient is a sodium phosphate buffer.

26. The composition of claim 25, wherein the pharmaceutically acceptable excipient is isotonic saline or water for injection.

27. A method for inhibiting expression of a PCSK9 gene in a hepatocyte cell, the method comprising introducing into a cell an effective amount of an RNAi agent of any one of claims 1-23 or the composition of any one of claims 24-26.

28. The method of claim 27, wherein the subject is a human subject.

29. The method of any one of claims 27-28, wherein the PCSK9 mRNA levels are reduced by at least about 50% in the hepatocyte cell or in the subject.

30. The method of any one of claims 27-29, wherein the PCSK9 protein levels are reduced by at least about 50% in the hepatocyte cell or in the subject.

31. A method of treating a PCSK9-related disease, disorder, or symptom, the method comprising administering to a human subject in need thereof a therapeutically effective amount of the composition of any one of claims 24-26.

32. The method of claim 31, wherein the disease is hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD).

33. The method of any one of claims 27-32, wherein the level of serum PCSK9 protein is decreased in the subject.

34. The method of any one of claims 27-33, wherein the RNAi agent is administered to a human subject at a dose of about 0.05 mg/kg to about 5.0 mg/kg of body weight of the human subject.

35. Use of the RNAi agent of any one of claims 1-23 or the composition according to any one of claims 24-26, for the treatment of a disease, disorder, or symptom that is mediated at least in part by a reduction in PCSK9 gene expression.

36. Use according to claim 35, wherein the disease is hypercholesterolemia, familial hypercholesterolemia including heterozygous familial hypercholesterolemia (HeFH) and homozygous familial hypercholesterolemia (HoFH), familial hypobetalipoproteinemia, hyperlipidemia, coronary artery disease, polygenic dyslipidemia, heart disease, cardiovascular disease (CVD) including clinical atherosclerotic cardiovascular disease (ASCVD), and/or other PCSK9-related disease.

37. Use of the RNAi agent of any one of claims 1-23 or the composition according to any one of claims 24-26, for the preparation of a pharmaceutical composition for treating a disease, disorder, or symptom that is mediated at least in part by a reduction in PCSK9 gene expression.

38. Use according to any one of claims 35-37, wherein the RNAi agent is administered to a human subject at a dose of about 0.05 mg/kg to about 5.0 mg/kg of body weight of the human subject.
